(19) United States
(12) Patent Application Publication
(10) Pub. No.: US 2023/0313162 A1
(43) Pub. Date: Oct. 5, 2023

(54) USE OF CRISPR-CAS ENDONUCLEASES FOR PLANT GENOME ENGINEERING

(71) Applicant: INARI AGRICULTURE TECHNOLOGY, INC., Cambridge, MA (US)

(72) Inventors: Neena Kenton Pyzocha, New Boston, MA (US); Adam Patrick Joyce, Stow, MA (US); Asa Budnick, Raleigh, NC (US)

(21) Appl. No.: 18/246,317

(22) PCT Filed: Sep. 21, 2021
(86) PCT No.: PCT/US21/51318

§ 371 Date: Mar. 22, 2023

Related U.S. Application Data
(60) Provisional application No. 63/082,275, filed on Sep. 23, 2020.
(60) Provisional application No. 63/201,821, filed on May 14, 2021.

(51) Int. Cl. C12N 9/22; C12N 15/10; C12N 15/82

(52) U.S. Cl. CPC C12N 9/22; C12N 15/102; C12N 15/8213; C12N 2310/20

(57) ABSTRACT

Use of CRISPR/Cas12d systems in eukaryotic organisms including plants and eukaryotic cells for genome engineering, and compositions used in such methods are disclosed.

### REFERENCE TO SEQUENCE LISTING SUBMITTED ELECTRONICALLY

The sequence listing contained in the file named “10082W01_ST25.txt”, which was created on Sep. 16, 2021 and electronically filed on Sep. 21, 2021, is incorporated herein by reference in its entirety.

### BACKGROUND

### 1. Technical Field

This disclosure relates to materials and methods for gene editing in eukaryotic cells, and particularly to methods for gene editing, that include for example and not limitation, using nucleic acid guided CRISPR/Cas12d systems.

### 2. Background and Related Art

The ability to precisely modify genetic material in eukaryotic cells enables a wide range of high value applications in medical, pharmaceutical, agricultural, basic research and other fields. Fundamentally, genome engineering provides this capability by introducing predefined genetic variation at specific locations in eukaryotic genomes, such as deleting, inserting, mutating, or substituting specific nucleic acid sequences. These alterations can be gene or location specific. However, a significant barrier to routine introduction of targeted genetic variation in eukaryotic cells is the absence of mutations, insertions, or rearrangements without a precursory break in the genome to stimulate changes. Targeted double-stranded breaks (DSBs) caused by expression of site-specific nucleases (SSNs) in plants, for example, can increase the frequency of homologous recombination (HR) at least two to three orders of magnitude (Puchta et al., Proc Natl Acad Sci USA 93:5055-5060, 1996). Thus, state of the art achievements in efficient gene editing for targeted mutagenesis, editing or insertions, are dependent on the ability to introduce genomic single- or double-strand breaks at specific locations in eukaryotic genomes. Efficient programmable endonuclease systems or SSNs are thereby fundamental for robust gene editing. Examples of SSNs that have been used for gene editing include homing endonucleases (also known as meganucleases), zinc-finger nucleases (ZFNs), transcription activator-like effector nucleases (TALENs), and clustered, regularly interspersed short palindromic repeat (CRISPR)/CRISPR-associated (Cas) nucleases. Among these systems, CRISPR/Cas is unique for its guide RNA component that enables target reprogramming that can be implemented more rapidly than the protein reengineering required to use the other systems.

The requirement for targeted introduction of chromosomal DSBs for efficient production of genetic variation renders SSNs essential in gene editing. Like CRISPR/Cas9 nucleases, CRISPR/Cas12d endonucleases (“CRISPR/Cas12d”) are involved in defense against foreign nucleic acids by using nucleic acid guides to specify a target sequence, which is then cleaved by the CRISPR/Cas12d protein component. Specifically, CRISPR/Cas12d can bind and cleave a target nucleic acid by forming a complex with a designed or synthetic nucleic acid-targeting nucleic acid, where cleavage of the target nucleic acid can introduce double-stranded breaks in the target nucleic acid. Also like the Cas9 system, the CRISPR/Cas12d nucleic acid guides provide a facile method for programming endonuclease sequence specificity.

Use of the CRISPR/Cas12d system in plants has not been previously demonstrated. Thus, this disclosure is based in part on the surprising discovery that CRISPR/Cas12d is active as an endonuclease at temperatures suitable for growth and culture of plants and plant cells and the further surprising discovery that the endonuclease can be used for gene editing in plant cells.

### SUMMARY

Embodiments of the present disclosure relate generally to methods and compositions for genome engineering and more specifically to use of the CRISPR/Cas12d system to perform genome engineering in eukaryotes including mammals, yeast, fungi, fish, and plants.

This disclosure is based in part on the discovery that nucleic acid-guided endonucleases of the CRISPR/Cas12d family can be used for eukaryotic (e.g. mammalian, yeast, fungal, fish, or plant) genome engineering. CRISPR/Cas12d endonuclease systems share the advantage of CRISPR/Cas9 systems because they can be programmed for target specificity with a simple single-stranded nucleic acid. Thus, CRISPR/Cas12d endonuclease systems can be used without limitation to make targeted modifications in heritable material of eukaryotic cells including targeted insertions and deletions, targeted sequence replacements, targeted small- and large-scale genomic rearrangements including inversions or chromosome rearrangements, targeted edits of endogenous sequence, and targeted integration of foreign sequence. These modifications can be made independently or as simultaneous or sequential multiplex modifications within the cell. Thus, many valuable traits can be introduced into eukaryotes (e.g. mammals, yeast, fungi, fish, or plants) with a CRISPR/Cas12d endonuclease system.

The disclosure also provides a method for modifying genetic material present in a eukaryotic (e.g. mammalian, yeast, fungal, fish. or plant) cell. The method can include delivering into the cell a nucleic acid-targeting nucleic acid that is targeted to a sequence of the cell's genetic material and a CRISPR/Cas12d endonuclease into a eukaryotic (e.g. mammalian, yeast, fungal, fish, or plant) cell. The nucleic acid-targeting nucleic acid can then direct the CRISPR/Cas12d endonuclease to the target site specified by the nucleic acid-targeting nucleic acid, where in some embodiments it creates breaks in the cell's genetic material at or near the target site specified by the nucleic acid-targeting nucleic acid. Repair of the breaks through the non-homologous end joining (NHEJ) or homologous recombination (HR) mediated pathways can result in targeted modifications in the genetic material of the eukaryotic (e.g. mammalian, yeast, fungal, fish, or plant) cell.

The nucleic acid-targeting nucleic acid and/or the CRISPR Cas12d endonuclease can be delivered together or separately into eukaryotic (e.g. mammalian, yeast, fungal, fish, or plant) cells via any suitable method including, for example and not limitation, by bacterial DNA-transfer such as Agrobacterium transformation, by microparticle bombardment, by polyethylene glycol (PEG) transformation, by transfection via e.g., a viral vector, by electroporation, or by another suitable method, including mechanical introduction methods. Alternatively, the nucleic acid-targeting nucleic acid and/or the CRISPR/Cas12d endonuclease can be delivered by Ensifer or in a T-DNA. Alternatively, an expression cassette for the CRISPR/Cas12d endonuclease can be stably integrated into the plant genome for heritable expression in the plant cell and its derivatives.

The use of CRISPR/Cas12d for eukaryotic genome engineering is described herein. As demonstrated, and as a general process, transient test systems such as protoplasts can be used to analyze, validate, and optimize nuclease activity at episomal and endogenous or transgenic chromosomal targets. Modifications can also be made in regenerative or reproductive tissues of plants, humans, and non-human animals such as non-human primates, bovine species, porcine species, murine species, canine species, feline species, equine species, rodents, ungulate species, and fish, enabling production of gene edited plants, plant lines, and non-human animals for basic research and agricultural applications.

Like other nucleic acid guided endonucleases, CRISPR/Cas12d SSNs usually require a minimum of two components for targeted mutagenesis in plant cells: a 5′-phosphorylated single-stranded guide-RNA and the CRISPR/Cas12d endonuclease protein. For targeted edits, insertions, or sequence replacements, a DNA template encoding the desired sequence changes can also be provided to the eukaryotic (e.g. mammalian, yeast, fungal, fish, or plant) cell to introduce changes either via the NHEJ or HR repair pathways. Successful editing events are most commonly detected by phenotypic changes (such as by knockout or introduction of a gene that results in a visible phenotype), by PCR-based methods (such as by enrichment PCR, PCR-digest, or T7EI or Surveyor endonuclease assays), or by targeted Next Generation Sequencing (NGS; also known as deep sequencing). For example, transgenic plants may encode a defective GUS:NPTII reporter gene. Also, PCR-based methods can be used to ascertain whether a genomic target site contains targeted mutations or donor sequence, and/or whether precise recombination has occurred at the 5′ and 3′ ends of the donor.

One advantage of the CRISPR Cas12d system is that it is functional at temperatures suitable for growth and culture of certain eukaryotes and eukaryotic cells, including plants and plant cells, such as for example and not limited to, about 20° C. to about 35° C., preferably about 23° C. to about 32° C., and most preferably about 25° C. to about 28° C.

In one aspect is provided a method for modifying expression of at least one chromosomal or extrachromosomal gene in a eukaryotic (e.g. mammalian, yeast, fungal, fish, or plant) cell, the method comprising introducing into the cell: (a) (i) a Clustered Regularly Interspersed Short Palindromic Repeats (CRISPR) RNA (crRNA) and a short-complementarity untranslated RNA (scoutRNA), or (ii) a chimeric cr/scoutRNA hybrid (sgRNA), wherein the crRNA or the sgRNA is targeted to a sequence within the gene or within an RNA molecule encoded by the gene; and (b) a CRISPR/Cas12d endonuclease molecule, wherein said CRISPR/Cas12d endonuclease may be capable of introducing a double stranded break or a single stranded break at or near the sequence to which the crRNA or sgRNA is targeted.

In some embodiments, the CRISPR/Cas12d endonuclease molecule is capable of introducing a single stranded break at or near the sequence to which the crRNA or sgRNA is targeted.

In some embodiments, the crRNA comprises a repeat sequence of about 11 nucleotides and a spacer sequence of about 18 nucleotides, wherein the spacer sequence interacts with the target nucleic acid. In some embodiments, a sgRNA comprises a scoutRNA with either a direct or indirect covalent (e.g. via a nucleotide or polynucleotide linker) linkage at the scoutRNA 3′ end to the 5′ end of a truncated DR (Direct Repeat) or full-length DR element, wherein the truncated DR or full-length DR element has a direct covalent linkage at its 3′ end to the 5′ end of a spacer element. Such sgRNAs thus comprise from 5′ to 3′ a scoutRNA, an optional indirect covalent linkage, a truncated or full-length DR element, and a spacer element.

In some embodiments, a sgRNA comprises a truncated DR or full-length DR a spacer element with either a direct or indirect covalent linkage at the DR 3′ end to a spacer element, which spacer element has either a direct or indirect covalent linkage at its 3′ end to a scoutRNA.

Such sgRNAs thus comprise from 5′ to 3′ a DR, a spacer element, an optional indirect covalent linkage which may include a DR, and a scoutRNA. In certain embodiments, such indirect covalent linkages of scoutRNA, truncated or full-length DR elements, and/or spacer elements is achieved with a nucleotide or polynucleotide linker. In some embodiments, a sgRNA comprises a spacer element with either a direct or indirect covalent linkage at the spacer element 3′ end to a scoutRNA, wherein the scoutRNA has either a direct or indirect covalent linkage at its 3′ end to at least a truncated DR (Direct Repeat) or full-length DR. Such sgRNAs thus comprise from 5′ to 3′ a spacer element, an optional indirect covalent linkage, a scoutRNA, an optional indirect covalent linkage, and a truncated or full-length DR element. In certain embodiments, such indirect covalent linkages of scoutRNA, truncated or full-length DR elements, and/or spacer elements is achieved with a nucleotide or polynucleotide linker. A nucleotide linker can comprise a single nucleotide (e.g., a ribonucleotide, deoxyribonucleotide, or unconventional/modified nucleotide). A nucleotide or polynucleotide linker used in an indirect covalent linkage can comprise about 1 to about 30 nucleotides (e.g., about 1, 2, 3, 4, or 5 to about 10, 15, 20, 25, or 30 nucleotides). In some embodiments, a polynucleotide linker may form structures such as pseudoknots or hairpins. In some embodiments, an indirect covalent linkage of a scoutRNA, truncated or full-length DR elements, and/or spacer elements can comprise a covalent bond which is not a phosphodiester bond (e.g., a phosphorothioate bond, thiophosphate bond, phosphoramidate bond, a thioether linker, or triazole linker). In some embodiments, the crRNA, scoutRNA, sgRNA, nucleotide linker, and/or polynucleotide linker comprises unconventional and/or modified nucleotides and/or comprises unconventional and/or modified backbone chemistries. In some embodiments, the scoutRNA or sgRNA comprises the RNA molecule of SEQ ID NO: 3, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 59, and variants thereof. In some embodiments, the sgRNA comprises the RNA molecule of SEQ ID NO: 4, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, SEQ ID NO: 59, and variants thereof. In some embodiments, the crRNA or scoutRNA or sgRNA comprises one or more modifications selected from the group consisting of locked nucleic acid (LNA) bases, internucleotide phosphorothioate bonds in the backbone, 2′-O-Methyl RNA bases, unlocked nucleic acid (UNA) bases, 5-Methyl dC bases, 5-hydroxybutynl-2′-deoxyuridine bases, 5-nitro indole bases, deoxyinosine bases, 8-aza-7-deazaguanosine bases, dideoxy-T at the 5′ end, inverted dT at the 3′ end, and dideoxycytidine at the 3′ end. Other examples of scout and sgRNA systems that can be adapted for use in the methods, compositions, and kits provided herein include those set forth in US20200255858, which is incorporated herein by reference in its entirety. Non-limiting examples of RNA molecules comprising scoutRNA/truncated DR or full-length DR sequences and chimeric cr/scoutRNA hybrid (sgRNA) molecules that can be used with suitable spacer sequences directed to DNA targets of interest and a Cas12d endonucleases, including the Cas12d endonuclease of SEQ ID NO: 1, include RNA molecules set forth in Table 1 and variants thereof. Such variants include those having 1, 2, 3 or more conservative nucleotide substitutions (e.g. purine for purine or pyrimidine for pyrimidine substitutions). Such variants RNA molecules set forth in Table 1 include those having paired sets of 2 substitutions which preserve base paired stem structures in the RNA molecules (e.g., two nucleotide which can base pair are substituted with 2 distinct nucleotides which can base pair). Such variants of RNA molecules set forth in Table 1 include RNA molecules comprising 1, 2, 3, or more unconventional and/or modified nucleotides and/or comprising 1, 2, 3, or more unconventional and/or modified backbone chemistries (e.g., a phosphorothioate bond, thiophosphate bond, and/or a phosphoramidate bond).

TABLE 1
RNA molecules comprising crRNAs, scoutRNAs and sgRNA molecules¹ for use with
Cas12d endonucleases including the Cas12d endonuclease of SEQ ID NO: 1.

| Description | Sequence (SEQ ID NO:) |
|---|---|
| Cas12d.15 scoutRNA | CUUAGUUAAGGAUGUUCCAGGUUCUUUCGGGAGCCUUGG |
| | CCUUCUCCCUUAACCUAUGCC (SEQ ID NO: 3) |
| Cas12d.15 crRNA | GCGAUGAAGGCNNNNNNNNNNNNNNNNNN (SEQ ID NO: 4) |
| sgRNA with altered | CUUAUUAAGGAUGUUCCAGGUUCUUUCGGGAGCCUUGGCC |
| scout and cr | UUCUCCCUUAACUAUCGCGAUGAAGGCNNNNNNNNNNNN |
| sequences | NNNNNN (SEQ ID NO: 5) |
| Simple fusion of the | CUUAGUUAAGGAUGUUCCAGGUUCUUUCGGGAGCCUUGG |
| scoutRNA with | CCUUCUCCCUUAACCUAUGCCGCGAUGAAGGC (SEQ ID NO: |
| truncated DR | 42) |
| Simple fusion of the | CUUAGUUAAGGAUGUUCCAGGUUCUUUCGGGAGCCUUGG |
| scoutRNA with | CCUUCUCCCUUAACCUAUGCCGCGAUGAAGGCNNNNNNNN |
| truncated DR with 18 | NNNNNNNNNN (SEQ ID NO: 43) |
| nt spacer sequence | |
| Simple fusion of the | CUUAGUUAAGGAUGUUCCAGGUUCUUUCGGGAGCCUUGG |
| scoutRNA with full 5′ | CCUUCUCCCUUAACCUAUGCCacccguaaagcagagcgaugaaggc |
| DR appended | (SEQ ID NO: 44) |
| between termini of | |
| scout and start of | |
| spacer | |
| Simple fusion of the | CUUAGUUAAGGAUGUUCCAGGUUCUUUCGGGAGCCUUGG |
| scoutRNA with full 5′ | CCUUCUCCCUUAACCUAUGCCacccguaaagcagagcgaugaaggcNN |
| DR appended | NNNNNNNNNNNNNNNN (SEQ ID NO: 45) |
| between termini of | |
| scout and start of | |
| spacer with 18 nt | |
| spacer | |
| scoutRNA extended | CUUAGUUAAGGAUGUUCCAGGUUCUUUCGGGAGCCUUGG |
| with additional | CCUUCUCCCUUAACCUAUGCCacuaaugauuaggaacacggGCGAUG |
| sequence from native | AAGGC (SEQ ID NO: 46) |
| locus as a linker, | |
| leading into truncated | |
| 5′ DR | |
| scoutRNA extended | CUUAGUUAAGGAUGUUCCAGGUUCUUUCGGGAGCCUUGG |
| with additional | CCUUCUCCCUUAACCUAUGCCacuaaugauuaggaacacggGCGAUG |
| sequence from native | AAGGCNNNNNNNNNNNNNNNNNN (SEQ ID NO: 47) |
| locus as a linker, | |
| leading into truncated | |
| 5′ DR and 18 nt | |
| spacer sequence | |
| scoutRNA extended | CUUAGUUAAGGAUGUUCCAGGUUCUUUCGGGAGCCUUGG |
| with additional | CCUUCUCCCUUAACCUAUGCCacuaaugauuaggaacacggacccguaaa |
| sequence from native | gcagagcgaugaaggc (SEQ ID NO: 48) |
| locus as a linker, | |
| leading into full 5′ | |
| DR | |
| scoutRNA extended | CUUAGUUAAGGAUGUUCCAGGUUCUUUCGGGAGCCUUGG |
| with additional | CCUUCUCCCUUAACCUAUGCCacuaaugauuaggaacacggacccguaaa |
| sequence from native | gcagagcgaugaaggcNNNNNNNNNNNNNNNNNN (SEQ ID NO: 49) |
| locus as a linker, | |
| leading into full 5′ | |
| DR and 18 nt spacer | |
| sequence | |
| scoutRNA extended | CUUAGUUAAGGAUGUUCCAGGUUCUUUCGGGAGCCUUGG |
| with additional | CCUUCUCCCUUAACCUAUGCCacuGCGGuAAuCCGCagaaGCG |
| sequence forming | AUGAAGGC (SEQ ID NO: 50) |
| hairpin, leading into | |
| truncated 5′ DR | |
| scoutRNA extended | CUUAGUUAAGGAUGUUCCAGGUUCUUUCGGGAGCCUUGG |
| with additional | CCUUCUCCCUUAACCUAUGCCacuGCGGuAAuCCGCagaaGCG |
| sequence forming | AUGAAGGCNNNNNNNNNNNNNNNNNN (SEQ ID NO: 51) |
| hairpin, leading into | |
| truncated 5′ DR and | |
| 18 nt spacer sequence | |
| scoutRNA extended | CUUAGUUAAGGAUGUUCCAGGUUCUUUCGGGAGCCUUGG |
| with additional | CCUUCUCCCUUAACCUAUGCCacuGCGGuAAuCCGCagaaacccg |
| sequence forming | uaaagcagagcgaugaaggc (SEQ ID NO: 52) |
| hairpin, leading into | |
| full 5′ DR | |
| scoutRNA extended | CUUAGUUAAGGAUGUUCCAGGUUCUUUCGGGAGCCUUGG |
| with additional | CCUUCUCCCUUAACCUAUGCCacuGCGGuAAuCCGCagaaacccg |
| sequence forming | uaaagcagagcgaugaaggcNNNNNNNNNNNN (SEQ ID NO: 53) |
| hairpin, leading into | |
| full 5′ DR and 18 nt | |
| spacer sequence | |
| scoutRNA extended | CUUAGUUAAGGAUGUUCCAGGUUCUUUCGGGAGCCUUGG |
| with additional | CCUUCUCCCUUAACCUAUGCCaaccagaauaaauccugguucuggcauau |
| sequence forming | accaggaaGCGAUGAAGGC (SEQ ID NO: 54) |
| pseudoknot, leading | |
| into truncated 5′ DR | |
| scoutRNA extended | CUUAGUUAAGGAUGUUCCAGGUUCUUUCGGGAGCCUUGG |
| with additional | CCUUCUCCCUUAACCUAUGCCaaccagaauaaauccugguucuggcauau |
| sequence forming | accaggaaGCGAUGAAGGCNNNNNNNNNNNNNNNNNN (SEQ ID |
| pseudoknot, leading | NO: 55) |
| into truncated 5′ DR | |
| and 18 nt spacer | |
| sequence | |
| scoutRNA extended | CUUAGUUAAGGAUGUUCCAGGUUCUUUCGGGAGCCUUGG |
| with additional | CCUUCUCCCUUAACCUAUGCCaaccagaauaaauccugguucuggcauau |
| sequence forming | accaggaaacccguaaagcagagcgaugaaggc (SEQ ID NO: 56) |
| pseudoknot, leading | |
| into full 5′ DR | |
| scoutRNA extended | CUUAGUUAAGGAUGUUCCAGGUUCUUUCGGGAGCCUUGG |
| with additional | CCUUCUCCCUUAACCUAUGCCaaccagaauaaauccugguucuggcauau |
| sequence forming | accaggaaacccguaaagcagagcgaugaaggcNNNNNNNNNNNNNNNNNN |
| pseudoknot, leading | (SEQ ID NO: 57) |
| into full 5′ DR and 18 | |
| nt spacer sequence | |
| transcript consisting | acccguaaagcagagcgaugaaggcNNNNNNNNNNNNNNNNNNacccguaa |
| of full DR, 18 nt | agcagagcgaugaaggcccgacuucgcugauaaaaauCUUAGUUAAGGAUGU |
| Spacer sequence, full | UCCAGGUUCUUUCGGGAGCCUUGGCCUUCUCCCUUAACCU |
| DR, 20 bp of native | AUGCC (SEQ ID NO: 58) |
| sequence 5′ of | |
| scoutRNA, and | |
| scoutRNA | |
| transcript consisting | acccguaaagcagagcgaugaaggcNNNNNNNNNNNNNNNNNNacccguaa |
| of full DR, 18 nt | agcagagcgaugaaggcgagcgaugaaggcacauuggccgacuucgcugaua |
| Spacer sequence, full | aaaauCUUAGUUAAGGAUGUUCCAGGUUCUUUCGGGAGCCUUGGCC |
| DR, 40 bp of native | UUCUCCCUUAACCUAUGCC (SEQ ID NO: 59) |
| sequence 5′ of | |
| scoutRNA, and | |
| scoutRNA | |

¹Spacer sequences are shown as 18-mers where the NNNNNNNNNNNNNNNNNN sequence can be any combination of nucleotides which are complementary or which can hybridize to a target nucleic acid. In certain embodiments, the sgRNA can comprise more than 18 nucleotides.

In some embodiments, the crRNA, scoutRNA or sgRNA is introduced into the cell as a DNA molecule encoding said RNA and is operably linked to a promoter directing production of said RNA in the cell. In some embodiments, DNAs encoding a scoutRNA or sgRNA comprising the RNA molecule of SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56 are provided. In some embodiments, DNAs encoding a sgRNA comprising the RNA molecule of SEQ ID NO: 5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59 are provided.

In some embodiments, the CRISPR/Cas12d endonuclease molecule comprises the amino acid sequence of SEQ ID NO: 1, a sequence having at least 85% sequence identity to SEQ ID NO: 1 a sequence having at least 90% sequence identity to SEQ ID NO: 1 or a sequence having at least 95% sequence identity to SEQ ID NO: 1.

In some embodiments, the CRISPR/Cas12d endonuclease molecule is a dCas12d, comprising one or more mutations of residues D775, E971, D1198, C1053, C1056, C1186, and C1191 of SEQ ID NO: 1, such as D775A, E971A, D1198A, C1053A, C1056A, C1186A, and C1191A of SEQ ID NO: 1.

In some embodiments, the CRISPR/Cas12d endonuclease molecule is modified so as to be active at a different temperature than its optimal temperature prior to modification. The modified CRISPR/Cas12d endonuclease molecule may be active at temperatures suitable for growth and culture of certain eukaryotes and eukaryotic cells, including plants or plant cells. The modified CRISPR/Cas12d endonuclease molecule may be active at a temperature from about 20° C. to about 35° C. The modified CRISPR/Cas12d endonuclease molecule may be active at a temperature from about 23° C. to about 32° C. The modified CRISPR/Cas12d endonuclease molecule may be active at a temperature from about 25° C. to about 28° C.

In some embodiments, the CRISPR/Cas12d endonuclease molecule is delivered to the cell as a DNA molecule comprising a CRISPR/Cas12d endonuclease coding sequence operably linked to a promoter directing production of said CRISPR/Cas12d endonuclease in the cell. The DNA molecule may be transiently present in the cell. The DNA molecule may be stably incorporated into the nuclear or plastidic genomic sequence of the cell or a progenitor cell, thereby providing heritable expression of the CRISPR/Cas12d endonuclease molecule. The DNA molecule may be stably incorporated into the chloroplast genome of the cell or a progenitor cell, thereby providing heritable expression of the CRISPR/Cas12d endonuclease molecule. In some embodiments, the promoter is selected from the group consisting of constitutive promoters, inducible promoters, and cell-type or tissue-type specific promoters. The promoter may be activated by alternative splicing of a suicide exon.

In some embodiments, the CRISPR/Cas12d endonuclease molecule is delivered to the cell as an mRNA molecule encoding said CRISPR/Cas12d endonuclease. In some embodiments, the CRISPR/Cas12d endonuclease molecule is delivered to the cell as a protein.

In some embodiments, the CRISPR/Cas12d endonuclease molecule has one or more localization signals, detection tags, detection reporters, and purification tags. In some embodiments, the CRISPR/Cas12d endonuclease molecule comprises one or more localization signals. The CRISPR/Cas12d endonuclease molecule may comprise at least one additional protein domain with enzymatic activity. The additional protein domain may have an enzymatic activity selected from the group consisting of exonuclease, helicase, repair of DNA double-stranded breaks, transcriptional (co-)activator, transcriptional (co-)repressor, methylase, demethylase, and any combinations thereof.

In some embodiments, the method comprises delivering a preassembled complex comprising the CRISPR/Cas12d endonuclease molecule loaded with the crRNA/scoutRNA or sgRNA prior to introduction into the cell.

In some embodiments, the DNA or RNA is delivered to the cell by a method selected from the group consisting of microparticle bombardment, polyethylene glycol (PEG) mediated transformation, electroporation, pollen-tube mediated introduction into zygotes, and delivery mediated by one or more cell-penetrating peptides (CPPs). The DNA may be delivered to the cell in a T-DNA. Delivery of DNA may be by bacteria-mediated transformation. Delivery of DNA may be via Agrobacterium or Ensifer.

In some embodiments, the DNA or RNA is delivered to the cell by a virus. The virus may be a geminivirus or a tobravirus. For mammalian cells, the viral vector can be an adenoviral vector, an adenovirus associated vector, a lentiviral vector, or a retroviral vector.

In some embodiments, the eukaryotic cell is a mammalian cell optionally selected from the group consisting of a human, non-human primate, bovine, porcine, murine, canine, feline, equine, rodent, and an ungulate cell. In some embodiments, the eukaryote is a mammal optionally selected from the group consisting of a human, non-human primate, bovine, porcine, murine, canine, feline, equine, rodent, and an ungulate species.

In some embodiments, the eukaryotic cell is a yeast cell optionally selected from the group consisting of a Saccharomyces sp., Candida sp., Endomycopsis sp., Brettanomyces sp., Candida sp., Cryptococcus sp., Debaromyces sp., Hanseniaspora sp., Hansenula sp., Kluyveromyces sp., Pichia sp., Rhodotorula sp., Torulaspora sp., Schizosaccharomyces sp., and Zygosaccharomyces sp. cell.

In some embodiments, the eukaryotic cell is a fungal cell optionally selected from the group consisting of an Aspergillus sp., Fusarium sp., Penicillium sp., Paecilomyces sp., Mucor sp., Rhizopus sp., and a Trichoderma sp. cell.

In some embodiments, the eukaryotic cell is a fish cell optionally selected from the group consisting of a salmonid, cichlid, silurid, and cyprinid cell. In some embodiments, the eukaryote is a fish optionally selected from the group consisting of a salmonid, cichlid, silurid, and cyprinid fish.

In some embodiments, the plant is monocotyledonous. In some embodiments, the plant is dicotyledonous.

In various embodiments, plant cell is derived from a species selected from the group consisting of Hordeum vulgar e, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, and any variety or subspecies belonging to one of the aforementioned plants.

In some embodiments, the target sequence is selected from the group consisting of an acetolactate synthase (ALS) gene, an enolpyruvylshikimate phosphate synthase gene (EPSPS) gene, male fertility genes, male sterility genes, female fertility genes, female sterility genes, male restorer genes, female restorer genes, genes associated with the traits of sterility, genes associated with the traits of fertility, genes associated with herbicide resistance, genes associated with herbicide tolerance, genes associated with fungal resistance, genes associated with viral resistance, genes associated with insect resistance, genes associated with drought tolerance, genes associated with chilling tolerance, genes associated with cold tolerance, genes associated with nitrogen use efficiency, genes associated with phosphorus use efficiency, genes associated with water use efficiency and genes associated with crop or biomass yield, and any mutants of such genes. The male sterility gene may be selected from the group consisting of MS45, MS26 and MSCA1.

In another aspect is provided a plant cell produced by the method of any of the above aspects or embodiments, and whole plants, or progeny thereof derived from the plant cell.

In yet another aspect is provided a composition comprising:
(a) (i) a Clustered Regularly Interspersed Short Palindromic Repeats (CRISPR) RNA (crRNA) and a short-complementarity untranslated RNA (scoutRNA), or
(ii) a chimeric cr/scoutRNA hybrid (sgRNA), wherein the crRNA or the sgRNA is targeted to a chromosomal or extrachromosomal plant gene sequence or within an RNA molecule encoded by said gene; and/or
(b) a CRISPR Cas12d endonuclease molecule, in which the CRISPR/Cas12d endonuclease is capable of introducing a double stranded break or a single stranded break at or near the sequence to which the crRNA or sgRNA is targeted at temperatures suitable for growth and culture of plants or plant cells.

In some embodiments, the crRNA comprises a repeat sequence of about 11 nucleotides and a spacer sequence of about 18 nucleotides; the spacer sequence interacts with the target nucleic acid.

In some embodiments, the crRNA or scoutRNA or sgRNA comprises unconventional and/or modified nucleotides and/or comprises unconventional and/or modified backbone chemistries. The crRNA, scoutRNA or sgRNA may comprise one or more modifications selected from the group consisting of locked nucleic acid (LNA) bases, internucleotide phosphorothioate bonds in the backbone, 2′-O-Methyl RNA bases, unlocked nucleic acid (UNA) bases, 5-Methyl dC bases, 5-hydroxybutynl-2′-deoxyuridine bases, 5-nitroindole bases, deoxyinosine bases, 8-aza-7-deazaguanosine bases, dideoxy-T at the 5′ end, inverted dT at the 3′ end, and dideoxycytidine at the 3′ end.

In some embodiments, the CRISPR/Cas12d endonuclease molecule comprises the amino acid sequence of SEQ ID NO: 1, a sequence having at least 85% sequence identity to SEQ ID NO: 1 a sequence having at least 90% sequence identity to SEQ ID NO: 1 or a sequence having at least 95% sequence identity to SEQ ID NO: 1.

In some embodiments, the CRISPR/Cas12d endonuclease molecule is modified so as to be active at a different temperature than its optimal temperature prior to modification. The modified CRISPR/Cas12d endonuclease molecule may be active at temperatures suitable for growth and culture of plants or plant cells. The modified CRISPR/Cas12d endonuclease molecule may be active at a temperature from about 20° C. to about 35° C. The modified CRISPR/Cas12d endonuclease molecule may be active at a temperature from about 23° C. to about 32° C. The modified CRISPR/Cas12d endonuclease molecule may be active at a temperature from about 25° C. to about 28° C.

In some embodiments, the CRISPR/Cas12d endonuclease molecule comprises one or more elements selected from the group consisting of localization signals, detection tags, detection reporters, and purification tags. In some embodiments, the CRISPR/Cas12d endonuclease molecule is modified to express nickase activity (nCas12d) or to have a nucleic acid targeting activity without any nickase or endonuclease activity (dCas12d).

In some embodiments, the CRISPR/Cas12d endonuclease molecule comprises at least one additional protein domain with enzymatic activity. The at least one additional protein domain can have an enzymatic activity selected from the group consisting of exonuclease, helicase, repair of DNA double-stranded breaks, transcriptional (co-)activator, transcriptional (co-) repressor, methylase, demethylase, and any combinations thereof.

In some embodiments, the target sequence is a plant sequence selected from the group consisting of an acetolactate synthase (ALS) gene, an enolpyruvylshikimate phosphate synthase gene (EPSPS) gene, male fertility genes, male sterility genes, female fertility genes, female sterility genes, male restorer genes, female restorer genes, genes associated with the traits of sterility, genes associated with the traits of fertility, genes associated with herbicide resistance, genes associated with herbicide tolerance, genes associated with fungal resistance, genes associated with viral resistance, genes associated with insect resistance, genes associated with drought tolerance, genes associated with chilling tolerance, genes associated with cold tolerance, genes associated with nitrogen use efficiency, genes associated with phosphorus use efficiency, genes associated with water use efficiency and genes associated with crop or biomass yield, and any mutants of such genes. The male sterility gene may be selected from the group consisting of MS45, MS26 and MSCA1.

In some embodiments, the plant is monocotyledonous. In some embodiments, the plant is dicotyledonous. The plant cell may be derived from a species selected from the group consisting of Hordeum vulgar e, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, and any variety or subspecies belonging to one of the aforementioned plants.

In another aspect is provided a kit comprising: (a) (i) a Clustered Regularly Interspersed Short Palindromic Repeats (CRISPR) RNA (crRNA) and a short-complementarity untranslated RNA (scoutRNA), or (ii) a chimeric cr/scoutRNA hybrid (sgRNA), wherein the crRNA or the sgRNA is targeted to a sequence within a plant gene or within an RNA molecule encoded by the gene; (b) a CRISPR Cas12d endonuclease molecule, wherein said CRISPR/Cas12d endonuclease is capable of introducing a double stranded break or a single stranded break at or near the sequence to which the crRNA or sgRNA is targeted at temperatures suitable for growth and culture of plants or plant cells, and optionally (c) instructions for use.

In another aspect is provided a kit comprising: (a) (i) a nucleic acid molecule encoding Clustered Regularly Interspersed Short Palindromic Repeats (CRISPR) RNA (crRNA) and a short-complementarity untranslated RNA (scoutRNA), or (ii) a nucleic acid molecule encoding a chimeric cr/scoutRNA hybrid (sgRNA), wherein the crRNA or the sgRNA is targeted to a sequence within a plant gene or within an RNA molecule encoded by the gene; (b) a nucleic acid molecule encoding CRISPR/Cas12d endonuclease molecule, wherein said CRISPR/Cas12d endonuclease is capable of introducing a double stranded break or a single stranded break at or near the sequence to which the crRNA or sgRNA is targeted at temperatures suitable for growth and culture of plants or plant cells, and optionally (c) instructions for use.

In another aspect is provided a kit comprising: (a) (i) a nucleic acid molecule encoding Clustered Regularly Interspersed Short Palindromic Repeats (CRISPR) RNA (crRNA) and a nucleic acid molecule encoding a short-complementarity untranslated RNA (scoutRNA), or (ii) a nucleic acid molecule encoding a chimeric cr/scoutRNA hybrid (sgRNA), wherein the crRNA or the sgRNA is targeted to a sequence within a plant gene or within an RNA molecule encoded by the gene; (b) a nucleic acid molecule encoding CRISPR/Cas12d endonuclease molecule, wherein said CRISPR Cas12d endonuclease is capable of introducing a double stranded break or a single stranded break at or near the sequence to which the crRNA or sgRNA is targeted at temperatures suitable for growth and culture of plants or plant cells, and optionally (c) instructions for use.

In another aspect, the disclosure provides a host cell comprising the CRISPR Cas12d endonuclease as described in any of the foregoing methods, and at least one nucleic acid-targeting nucleic acid as described in any of the foregoing methods.

In yet another aspect, the disclosure provides a vector comprising a nucleic acid encoding the CRISPR/Cas12d endonuclease as described in any of the foregoing methods and at least one nucleic acid-targeting nucleic acid as described in any of the foregoing methods.

In a further aspect, the disclosure provides a method for treating a disease and/or condition and/or preventing insect infection/infestation in a plant comprising modifying chromosomal or extrachromosomal genetic material of said plant by use of any of the foregoing methods.

Non-limiting examples of the diseases and/or conditions treatable include Anthracnose Stalk Rot, Aspergillus Ear Rot, Common Corn Ear Rots, Corn Ear Rots (Uncommon), Common Rust of Corn, Diplodia Ear Rot, Diplodia Leaf Streak, Diplodia Stalk Rot, Downy Mildew, Eyespot, Fusarium Ear Rot, Fusarium Stalk Rot, Gibberella Ear Rot, Gibberella Stalk Rot, Goss's Wilt and Leaf Blight, Gray Leaf Spot, Head Smut, Northern Corn Leaf Blight, Physoderma Brown Spot, Pythium, Southern Leaf Blight, Southern Rust, and Stewart's Bacterial Wilt and Blight, and combinations thereof.

Non-limiting examples of the insects causing, directly or indirectly, diseases and/or conditions treatable include Armyworm, Asiatic Garden Beetle, Black Cutworm, Brown Marmorated Stink Bug, Brown Stink Bug, Common Stalk Borer, Corn Billbugs, Corn Earworm, Corn Leaf Aphid, Corn Rootworm, Corn Rootworm Silk Feeding, European Corn Borer, Fall Armyworm, Grape Colaspis, Hop Vine Borer, Japanese Beetle, Scouting for Fall Armyworm, Seedcorn Beetle, Seedcorn Maggot, Southern Corn Leaf Beetle, Southwestern Corn Borer, Spider Mite, Sugarcane Beetle, Western Bean Cutworm, White Grub, and Wireworms, and combinations thereof. The invented methods are also suitable for preventing infections and/or infestations of a plant by any such insect(s).

In another aspect, the disclosure provides a method for affecting at least one trait in a plant selected from the group consisting of sterility, fertility, herbicide resistance, herbicide tolerance, fungal resistance, viral resistance, insect resistance, drought tolerance, chilling tolerance, or cold tolerance, nitrogen use efficiency, phosphorus use efficiency, water use efficiency and crop or biomass yield, said method comprising modifying chromosomal or extrachromosomal genetic material of said plant by use of any of the foregoing methods.

In another aspect, the disclosure relates to a chimeric cr/scoutRNA hybrid (sgRNA) nucleic acid comprising or a DNA encoding an sgRNA for a Cas12d nuclease, wherein the sgRNA comprises SEQ ID NO: 5.

In another aspect, the disclosure relates to a dCas12 molecule comprising a mutation of one or more of residues D775, E971, D1198, C1053, C1056, C1186, and C1191 of SEQ ID NO: 1, such as D775A, E971A, D1198A, C1053A, C1056A, C1186A, and C1191A of SEQ ID NO: 1.

The reduced size of these CRISPR/Cas12d endonucleases compared to the CRISPR/Cas9 system provides at least the following advantages: simplification of cloning and vector assembly, increased expression levels of the nuclease in cells, and reducing the challenge in expressing the protein from highly size-sensitive platforms such as viruses, including either DNA or RNA viruses.

These and other objects, features and advantages of the present disclosure will become more apparent upon reading the following specification in conjunction with the accompanying description and claims.

### BRIEF DESCRIPTION OF DRAWINGS

FIGS. 1A, B. FIG. 1A shows Cas12d.15 scoutRNA (top; SEQ ID NO: 3) and Cas12d.15 crRNA (bottom (SEQ ID NO: 4) in an RNA fold predicted using an RNA secondary structure folding algorithm (Andronescu et al., Bioinformatics, Volume 23, Issue 13, July 2007, Pages i19-i28). FIG. 1B shows Cas12d.15 scoutRNA and Cas12d.15 crRNA fused together to create a single guide RNA (sgRNA) for use with Cas12d.15 (SEQ ID NO: 5). The sgRNA keeps the same RNA secondary structure as individually complexed Cas12d.15 scoutRNA and Cas12d.15 crRNA.

FIG. 2 shows a map of the pIN2670 vector.

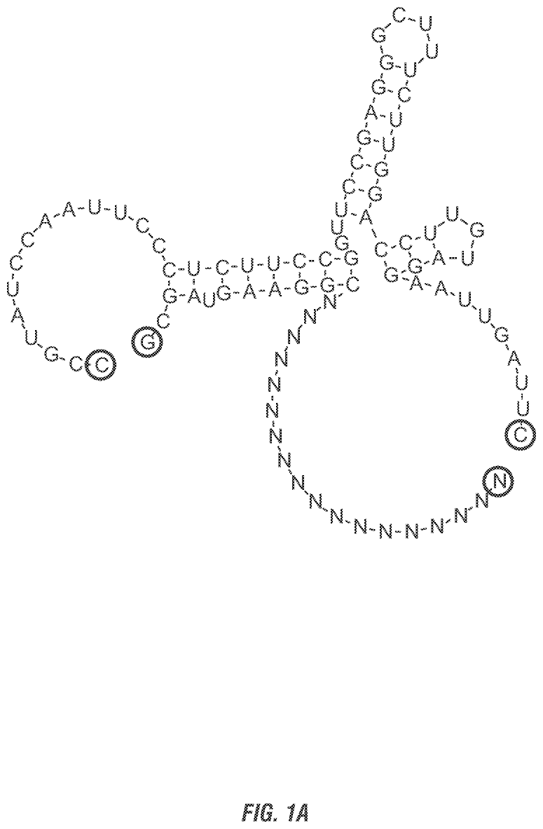

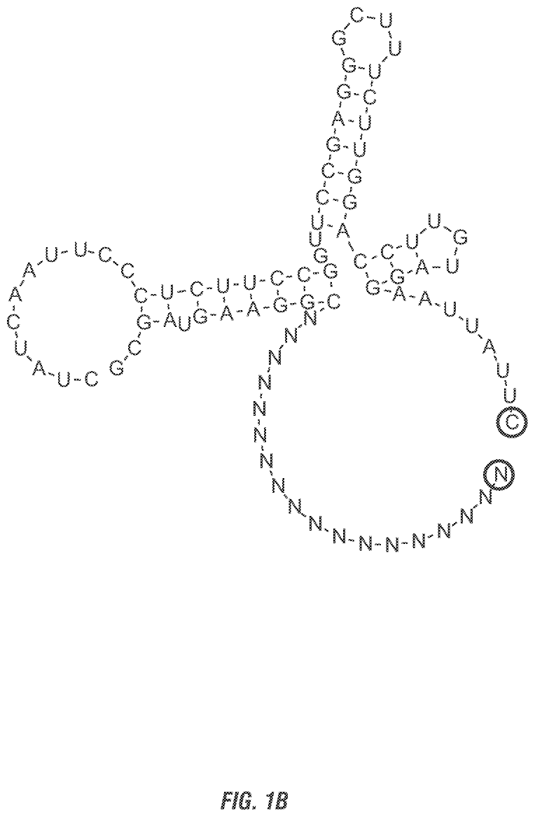

### DETAILED DESCRIPTION OF THE DISCLOSURE

To facilitate an understanding of the principles and features of the various embodiments of the disclosure, various illustrative embodiments are explained below. Although exemplary embodiments of the disclosure are explained in detail, it is to be understood that other embodiments are contemplated. Accordingly, it is not intended that the disclosure be limited in its scope to the details of construction and arrangement of components set forth in the following description or examples. The disclosure is capable of other embodiments and of being practiced or carried out in various ways. Also, in describing the exemplary embodiments, specific terminology will be resorted to for the sake of clarity.

It must also be noted that, as used in the specification and the appended claims, the singular forms “a”, “an”, and “the” include plural references unless the context clearly dictates otherwise. For example, reference to a component is intended also to include composition of a plurality of components. References to a composition containing “a” constituent is intended to include other constituents in addition to the one named. In other words, the terms “a”, “an”, and “the” do not denote a limitation of quantity, but rather denote the presence of “at least one” of the referenced item.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to ±10%, more preferably up to +5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first”, “second”, and the like, “primary”, “secondary”, and the like, do not denote any order, quantity, or importance, but rather are used to distinguish one element from another.

It is noted that terms like “specifically”, “preferably”, “typically”, “generally”, and “often” are not utilized herein to limit the scope of the claimed disclosure or to imply that certain features are critical, essential, or even important to the structure or function of the claimed disclosure. Rather, these terms are merely intended to highlight alternative or additional features that may or may not be utilized in a particular embodiment of the present disclosure. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present disclosure are intended to be illustrative and not restrictive. Many suitable materials that would perform the same or a similar function as the materials described herein are intended to be embraced within the scope of the disclosure. Such other materials not described herein can include, but are not limited to, materials that are developed after the time of the development of the disclosure, for example.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et ah, 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Definitions

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA that is single- or double-stranded, optionally containing synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine.

As used herein, the terms “CRISPR/Cas12d”, “Cas12d”, “Cas12d endonuclease” and CRISPR/Cas12d endonuclease” can be used interchangeably. A CRISPR Cas12d or a Cas12d can refer to any modified (e.g., shortened, mutated, lengthened) polypeptide sequence or homologue of the CRISPR/Cas12d, including variant, modified, fusion (as defined herein), and/or enzymatically inactive forms of the CRISPR/Cas12d. A CRISPR Cas12d can be codon optimized. A CRISPR/Cas12d can be a codon-optimized homologue of a CRISPR/Cas12d. A CRISPR Cas12d can be enzymatically inactive, partially active, constitutively active, fully active, inducibly active, active at different temperatures, and/or more active (e.g., more than the wild type homologue of the protein or polypeptide). In some instances, the CRISPR/Cas12d (e.g., variant, mutated, and/or enzymatically inactive CRISPR/Cas12d) can target a target nucleic acid. The CRISPR/Cas12d can associate with a short targeting or guide nucleic acid that provides specificity for a target nucleic acid to be cleaved by the protein's endo nuclease activity. The CRISPR/Cas12d can be provided separately or in a complex wherein it is pre-associated with the targeting or guide nucleic acid. In some instances, the CRISPR/Cas12d can be a fusion protein as described herein, for example CRISPR/Cas12d fused to mNeonGreen.

In some embodiments, the sequence TR is located 5′ to a protospacer sequence in the target.

CRISPR/Cas12d efficiently creates site-specific DNA double-strand breaks when loaded with the guide-RNA. The CRISPR/Cas12d is active at temperatures that are suitable for genome engineering in plants. Exemplary amino acid sequence of CRISPR/Cas12d are provided herein as SEQ ID NO: 1. The CRISPR/Cas12d is functional at a temperature range that is also suitable for growth and culture of plants and plant cells, such as for example and not limitation, about 20° C. to about 35° C., preferably about 23° C. to about 32° C., and most preferably about 25° C. to about 28° C. The CRISPR/Cas12d may be used in any of the embodiments described herein.

As used herein, “spacer”, “nucleic acid-targeting nucleic acid” or “nucleic acid-targeting guide nucleic acid” or “guide-RNA” are used interchangeably and can refer to a nucleic acid that can bind a CRISPR/Cas12d protein of the disclosure and hybridize with a target nucleic acid. A nucleic acid-targeting nucleic acid can be RNA, including, without limitation, one or more single-stranded RNA. CRISPR/Cas12d may be guided by a scoutRNA and a crRNA. CRISPR/Cas12d may be guided by a sgRNA (single-guide RNA) in which a scoutRNA is joined to crRNA. Transcriptional processing of crRNA may result in inclusion of about 10-20, for example 11, nucleotides of a repeat sequence and about 18 nucleotides (e.g. 16, 17, 19, or 20) of adjacent spacer sequence.

The nucleic acid-targeting nucleic acid can bind to a target nucleic acid site-specifically. A portion of the nucleic acid-targeting nucleic acid can be complementary to a portion of a target nucleic acid. A nucleic acid-targeting nucleic acid can comprise a segment that can be referred to as a “nucleic acid-targeting segment.” A nucleic acid-targeting nucleic acid can comprise a segment that can be referred to as a “protein-binding segment.” The nucleic acid-targeting segment and the protein-binding segment can be the same segment of the nucleic acid-targeting nucleic acid. The nucleic acid-targeting nucleic acid may contain modified nucleotides, a modified backbone, or both. The nucleic acid-targeting nucleic acid may comprise a peptide nucleic acid (PNA).

As used herein, “donor polynucleotide” can refer to a nucleic acid that can be integrated into a site during genome engineering, target nucleic acid engineering, or during any other method of the disclosure.

As used herein, “fusion” can refer to a protein and/or nucleic acid comprising one or more non-native sequences (e.g., moieties). A fusion can be at the N-terminal or C-terminal end of the modified protein, or both. A fusion can be a transcriptional and/or translational fusion. A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the CRISPR/Cas12d (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa Fluor® dyes, Cyanine3 dye, Cyanine5 dye. The fusion can provide for increased or decreased stability. In some embodiments, a fusion can comprise a detectable label, including a moiety that can provide a detectable signal. Suitable detectable labels and/or moieties that can provide a detectable signal can include, but are not limited to, an enzyme, a radioisotope, a member of a specific binding pair; a fluorophore; a fluorescent reporter or fluorescent protein; a quantum dot; and the like. A fusion can comprise a member of a FRET pair, or a fluorophore/quantum dot donor/acceptor pair. A fusion can comprise an enzyme. Suitable enzymes can include, but are not limited to, horse radish peroxidase, luciferase, beta-galactosidase, and the like. A fusion can comprise a fluorescent protein. Suitable fluorescent proteins can include, but are not limited to, a green fluorescent protein (GFP) (e.g., a GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum) any of a variety of fluorescent and colored proteins. A fusion can comprise a nanoparticle. Suitable nanoparticles can include fluorescent or luminescent nanoparticles, and magnetic nanoparticles. Any optical or magnetic property or characteristic of the nanoparticle(s) can be detected.

A fusion can comprise a helicase, a nuclease (e.g., Fokl), an endonuclease, an exonuclease (e.g., a 5′ exonuclease and/or 3′ exonuclease), a ligase, a nickase, a nuclease-helicase (e.g., Cas3), a DNA methyltransferase (e.g., Dam), or DNA demethylase, a histone methyltransferase, a histone demethylase, an acetylase (including for example and not limitation, a histone acetylase), a deacetylase (including for example and not limitation, a histone deacetylase), a phosphatase, a kinase, a transcription (co-) activator, a transcription (co-) factor, an RNA polymerase subunit, a transcription repressor, a DNA binding protein, a DNA structuring protein, a long noncoding RNA, a DNA repair protein (e.g., a protein involved in repair of either single and/or double-stranded breaks, e.g., proteins involved in base excision repair, nucleotide excision repair, mismatch repair, NHEJ, HR, microhomology-mediated end joining (MMEJ), and/or alternative non-homologous end-joining (ANHEJ), such as for example and not limitation, HR regulators and HR complex assembly signals), a marker protein, a reporter protein, a fluorescent protein, a ligand binding protein (e.g., mCherry or a heavy metal binding protein), a signal peptide (e.g., Tat-signal sequence), a targeting protein or peptide, a subcellular localization sequence (e.g., nuclear localization sequence, a chloroplast localization sequence), and/or an antibody epitope, or any combination thereof.

As used herein, “genome engineering” can refer to a process of modifying a target nucleic acid. Genome engineering can refer to the integration of non-native nucleic acid into native nucleic acid. Genome engineering can refer to the targeting of a CRISPR/Cas12d and a nucleic acid-targeting nucleic acid to a target nucleic acid. Genome engineering can refer to the cleavage of a target nucleic acid, and the rejoining of the target nucleic acid without an integration of an exogenous sequence in the target nucleic acid, or a deletion in the target nucleic acid. The native nucleic acid can comprise a gene. The non-native nucleic acid can comprise a donor polynucleotide. The endonuclease can create targeted DNA double-strand breaks at the desired locus (or loci), and the plant cell can repair the double-strand break using the donor polynucleotide, thereby incorporating the modification stably into the plant genome.

In the methods of the disclosure, CRISPR/Cas12d proteins, or complexes thereof, can introduce double-stranded breaks in a nucleic acid, (e.g. genomic DNA). The double-stranded break can stimulate a cell's endogenous DNA-repair pathways (e.g., homologous recombination (HR) and/or non-homologous end joining (NHEJ), or A-NHEJ (alternative non-homologous end-joining)). Mutations, deletions, alterations, and integrations of foreign, exogenous, and/or alternative nucleic acid can be introduced into the site of the double-stranded DNA break.

As used herein, the term “isolated” can refer to a nucleic acid or polypeptide that, by the hand of a human, exists apart from its native environment and is therefore not a product of nature. Isolated can mean substantially pure. An isolated nucleic acid or polypeptide can exist in a purified form and/or can exist in a non-native environment such as, for example, in a transgenic cell.

As used herein, “non-native” can refer to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide. A non-native sequence can refer to a 3′ hybridizing extension sequence.

As used herein, “nucleotide” can generally refer to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example and not limitation, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddlTP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited to fluorescein, 5-carboxyfluorescein (FAM), 27′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Tex. Red, Cyanine and 5-(2′-aminoethyl)aminonaphthalene-1-sulfonic acid (EDANS).

As used herein, “recombinant” can refer to sequence that originates from a source foreign to the particular host (e.g., cell) or, if from the same source, is modified from its original form. A recombinant nucleic acid in a cell can include a nucleic acid that is endogenous to the particular cell but has been modified through, for example, the use of site-directed mutagenesis. The term “recombinant” can include non-naturally occurring multiple copies of a naturally occurring DNA sequence. Thus, the term “recombinant” can refer to a nucleic acid that is foreign or heterologous to the cell, or homologous to the cell but in a position or form within the cell in which the nucleic acid is not ordinarily found. Similarly, when used in the context of a polypeptide or amino acid sequence, an exogenous polypeptide or amino acid sequence can be a polypeptide or amino acid sequence that originates from a source foreign to the particular cell or, if from the same source, is modified from its original form.

As used herein, the term “specific” can refer to interaction of two molecules where one of the molecules through, for example chemical or physical means, specifically binds to the second molecule. Exemplary specific binding interactions can refer to antigen-antibody binding, avidin-biotin binding, carbohydrates and lectins, complementary nucleic acid sequences (e.g., hybridizing), complementary peptide sequences including those formed by recombinant methods, effector and receptor molecules, enzyme cofactors and enzymes, enzyme inhibitors and enzymes, and the like. “Non-specific” can refer to an interaction between two molecules that is not specific.

As used herein, “target nucleic acid” or “target site” can generally refer to a target nucleic acid to be targeted in the methods of the disclosure. A target nucleic acid can refer to a nuclear chromosomal/genomic sequence or an extrachromosomal sequence, (e.g., an episomal sequence, a minicircle sequence, a mitochondrial sequence, a chloroplast sequence, a protoplast sequence, a plastid sequence, etc.) A target nucleic acid can be DNA. A target nucleic acid can be single-stranded DNA. A target nucleic acid can be double-stranded DNA. A target nucleic acid can be single-stranded or double-stranded RNA. A target nucleic acid can herein be used interchangeably with “target nucleotide sequence” and/or “target polynucleotide”.

As used herein, “sequence identity” or “identity” in the context of nucleic acid or polypeptide sequences refers to the nucleic acid bases or amino acid residues in two sequences that are the same when aligned for maximum correspondence over a specified comparison window.

As used herein, the term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%.

As used herein, the term “plant” refers to whole plants, plant organs, plant tissues, seeds, plant cells, seeds and progeny of the same. Plant cells include, without limitation, cells from seeds, suspension cultures, embryos, zygotes, meristematic regions, callus tissue, leaves, roots, shoots, gametophytes, protoplasts, plastids, sporophytes, pollen and microspores. Plant parts include differentiated and undifferentiated tissues including, but not limited to roots, stems, shoots, leaves, pollen, seeds, flowers, parts consumable by humans and/or other mammals (e.g., rice grains, corn cobs, tubers), tumor tissue and various forms of cells and culture (e.g., single cells, protoplasts, plastids, embryos, zygotes, and callus tissue).

“Plant tissue” encompasses plant cells and may be in a plant or in a plant organ, tissue or cell culture. A plant tissue also refers to any clone of such a plant, seed, progeny, propagule whether generated sexually or asexually, and descendants of any of these, such as cuttings or seed. The term “plant organ” refers to plant tissue or a group of tissues that constitute a morphologically and functionally distinct part of a plant. The term “genome” refers to the entire complement of genetic material (genes and non-coding sequences) that is present in each cell of an organism, or virus or organelle; and/or a complete set of chromosomes inherited as a (haploid) unit from one parent. “Progeny” comprises any subsequent generation of a plant.

As used herein, the term “transgenic plant” includes, for example, a plant which comprises within its genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A transgenic plant can also comprise more than one heterologous polynucleotide within its genome. Each heterologous polynucleotide may confer a different trait to the transgenic plant. A heterologous polynucleotide can include a sequence that originates from a foreign species, or, if from the same species, can be substantially modified from its native form. Transgenic can include any cell, cell line, callus, tissue, plant part or plant, the genotype of which has been altered by the presence of heterologous nucleic acid including those transgenics initially so altered as well as those created by sexual crosses or asexual propagation from the initial transgenic. The alterations of the genome (chromosomal or extra-chromosomal) by conventional plant breeding methods, by the genome editing procedure described herein that does not result in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as transgenic.

In certain embodiments of the disclosure, a fertile plant is a plant that produces viable male and female gametes and is self-fertile. Such a self-fertile plant can produce a progeny plant without the contribution from any other plant of a gamete and the genetic material contained therein. Other embodiments of the disclosure can involve the use of a plant that is not self-fertile because the plant does not produce male gametes, or female gametes, or both, that are viable or otherwise capable of fertilization. As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

As used herein, the terms “plasmid”, “vector” and “cassette” refer to an extra-chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector containing a gene and having elements in addition to the gene that facilitates transformation of a particular host cell. “Expression cassette” refers to a specific vector containing a gene and having elements in addition to the gene that allow for expression of that gene in a host.

The expression cassette for stable integration into the genome of a plant cell may contain one or more of the following elements: a promoter element that can be used to express the RNA and/or Cas12d enzyme in a plant cell; a 5′ untranslated region to enhance expression; an intron element to further enhance expression in certain cells, such as monocot cells; a multiple-cloning site to provide convenient restriction sites for inserting the guide RNA and/or the Cas12d gene sequences and other desired elements; and a 3′ untranslated region to provide for efficient termination of the expressed transcript.

The terms “recombinant DNA molecule”, “recombinant construct”, “expression construct”, “construct”, “construct”, and “recombinant DNA construct” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. A T7 vector (pSF-T7) can be used to allow production of capped RNA for transfection into cells. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:241 1-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis. Other techniques such as SI RNase protection, primer-extension, in situ hybridization, enzyme staining, and immunostaining also can be used to detect the presence or expression of polypeptides and/or polynucleotides.

As used herein, the term “expression” refers to the production of a functional end-product (e.g., an mRNA, guide RNA, or a protein) in either precursor or mature form.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, the term “mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

As used herein, the term “stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or other DNA-containing organelle, of a host organism resulting in gene expression without integration or stable inheritance. Host organisms containing the transformed nucleic acid fragments are referred to as “transgenic” organisms. The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different genes of interest.

As used herein, the terms “crossed” or “cross” or “crossing” means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

As used herein, the term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

As used herein, the term “hybridized” means hybridizing under conventional conditions, as described in Sambrook et al. (1989), preferably under stringent conditions. Stringent hybridization conditions are for example and not limitation: hybridizing in 4×SSC at 65° C. and subsequent multiple washing in 0.1×SSC at 65° C. for a total of approximately one hour. Less stringent hybridization conditions are for example and not limitation: hybridizing in 4×SSC at 37° C. and subsequent multiple washing in 1×SSC at room temperature. “Stringent hybridization conditions” can also mean for example and not limitation: hybridizing at 68° C. in 0.25 M sodium phosphate, pH 7.2, 7% SDS, 1 mM EDTA and 1% BSA for 16 hours and subsequent two times washing with 2×SSC and 0.1% SDS at 68° C.

### CRISPR/Cas12d Endonucleases of the Disclosure

CRISPR/Cas12d may introduce double-stranded breaks in the target nucleic acid, (e.g. genomic DNA). The double-stranded break can stimulate a cell's endogenous DNA-repair pathways (e.g., HR, NHEJ, A-NHEJ, or MMEJ). NHEJ can repair cleaved target nucleic acid without the need for a homologous template. This can result in deletions of the target nucleic acid. Homologous recombination (HR) can occur with a homologous template. The homologous template can comprise sequences that are homologous to sequences flanking the target nucleic acid cleavage site. After a target nucleic acid is cleaved by CRISPR/Cas12d, the site of cleavage can be destroyed (e.g., the site may not be accessible for another round of cleavage with the original nucleic acid-targeting nucleic acid and CRISPR/Cas12d).

A CRISPR/Cas12d can comprise a nucleic acid-binding domain. The nucleic acid-binding domain can comprise a region that contacts a nucleic acid. A nucleic acid-binding domain can comprise a nucleic acid. A nucleic acid-binding domain can comprise a proteinaceous material. A nucleic acid-binding domain can comprise nucleic acid and a proteinaceous material. A nucleic acid-binding domain can comprise DNA. A nucleic acid-binding domain can comprise single-stranded DNA. Examples of nucleic acid-binding domains can include, but are not limited to, a helix-turn-helix domain, a zinc finger domain, a leucine zipper (bZIP) domain, a winged helix domain, a winged helix turn helix domain, a helix-loop-helix domain, an HMG-box domain, a Wor3 domain, an immunoglobulin domain, a B3 domain, and a TALE domain. A nucleic acid-binding domain can be a domain of a CRISPR/Cas12d protein. A CRISPR/Cas12d protein can bind RNA or DNA, a DNA/RNA heteroduplex, or both RNA and DNA. A CRISPR/Cas12d protein can cleave RNA, or DNA, a DNA/RNA heteroduplex, or both RNA and DNA. In some instances, a CRISPR/Cas12d protein binds a DNA and cleaves the DNA. In some instances, the CRISPR/Cas12d protein binds a double-stranded DNA and cleaves a double-stranded DNA. In some instances, two or more nucleic acid-binding domains can be linked together. Linking a plurality of nucleic acid-binding domains together can provide increased polynucleotide targeting specificity. Two or more nucleic acid-binding domains can be linked via one or more linkers. The linker can be a flexible linker. Linkers can comprise 1 2, 3, 4, 5, 6, 7, 8, 9, 10, 11 12, 13, 14, 15, 16, 17, 18, 19, 20, 21 22, 23, 24, 25, 30, 35, 40 or more amino acids in length. The linker domain may comprise glycine and/or serine, and in some embodiments may consist of or may consist essentially of glycine and/or serine. Linkers can be a nucleic acid linker which can comprise nucleotides. A nucleic acid linker can link two DNA-binding domains together. A nucleic acid linker can be at most 5, 10, 15, 20, 25, 30, 35, 40, 45, or 50 or more nucleotides in length. A nucleic acid linker can be at least 5, 10, 15, 30, 35, 40, 45, or 50 or more nucleotides in length.

Nucleic acid-binding domains can bind to nucleic acid sequences. Nucleic acid binding domains can bind to nucleic acids through hybridization. Nucleic acid-binding domains can be engineered (e.g., engineered to hybridize to a sequence in a genome). A nucleic acid-binding domain can be engineered by molecular cloning techniques (e.g., directed evolution, site-specific mutation, and rational mutagenesis).

A CRISPR/Cas12d can comprise a nucleic acid-cleaving domain. The nucleic acid-cleaving domain can be a nucleic acid-cleaving domain from any nucleic acid-cleaving protein. The nucleic acid-cleaving domain can originate from a nuclease. Suitable nucleic acid-cleaving domains include the nucleic acid-cleaving domain of endonucleases (e.g., AP endonuclease, RecBCD endonuclease, T7 endonuclease, T4 endonuclease IV, Bal 31 endonuclease, Endonuclease 1 (endo I), Micrococcal nuclease, Endonuclease II (endo VI, exo III)), exonucleases, restriction nucleases, endoribonucleases, exoribonucleases, RNases (e.g., RNAse I, II, or III). A nucleic acid-binding domain can be a domain of a CRISPR/Cas12d protein. A CRISPR/Cas12d protein can bind RNA or DNA, or both RNA and DNA. A CRISPR/Cas12d protein can cleave RNA, or DNA, or both RNA and DNA. In some instances, a CRISPR/Cas12d protein binds a DNA and cleaves the DNA. In some instances, the CRISPR/Cas12d protein binds a double-stranded DNA and cleaves a double-stranded DNA. In some instances, the nucleic acid-cleaving domain can originate from the Fokl endonuclease. A CRISPR/Cas12d can comprise a plurality of nucleic acid-cleaving domains. Nucleic acid-cleaving domains can be linked together. Two or more nucleic acid-cleaving domains can be linked via a linker. In some embodiments, the linker can be a flexible linker as described herein. Linkers can comprise 1, 2, 3, 4, 5, 6, 7, 8, 9, 10, 1 1, 12, 13, 14, 15, 16, 17, 18, 19, 20, 21, 22, 23, 24, 25, 30, 35, 40 or more amino acids in length. In some embodiments, a CRISPR/Cas12d can comprise the plurality of nucleic acid-cleaving domains.

CRISPR/Cas12d can introduce double-stranded breaks in nucleic acid, (e.g., genomic DNA). The double-stranded break can stimulate a cell's endogenous DNA-repair pathways (e.g. homologous recombination and non-homologous end joining (NHEJ) or alternative non-homologues end joining (A-NHEJ)). NHEJ can repair cleaved target nucleic acid without the need for a homologous template. This can result in deletions of the target nucleic acid. Homologous recombination (HR) can occur with a homologous template. The homologous template can comprise sequences that are homologous to sequences flanking the target nucleic acid cleavage site. After a target nucleic acid is cleaved by a CRISPR/Cas12d the site of cleavage can be destroyed (e.g., the site may not be accessible for another round of cleavage with the original nucleic acid-targeting nucleic acid and CRISPR/Cas12d).

In some cases, homologous recombination can insert an exogenous polynucleotide sequence into the target nucleic acid cleavage site. An exogenous polynucleotide sequence can be called a donor polynucleotide. In some instances of the methods of the disclosure the donor polynucleotide, a portion of the donor polynucleotide, a copy of the donor polynucleotide, or a portion of a copy of the donor polynucleotide can be inserted into the target nucleic acid cleavage site. A donor polynucleotide can be an exogenous polynucleotide sequence. A donor polynucleotide can be a sequence that does not naturally occur at the target nucleic acid cleavage site. A vector can comprise a donor polynucleotide. The modifications of the target DNA due to NHEJ and/or HR can lead to, for example, mutations, deletions, alterations, integrations, gene correction, gene replacement, gene tagging, transgene insertion, nucleotide deletion, gene disruption, and/or gene mutation. The process of integrating non-native nucleic acid into genomic DNA can be referred to as genome engineering.

In some cases, the CRISPR/Cas12d can comprise an amino acid sequence having at most 10%, at most 15%, at most 20%, at most 30%, at most 40%, at most 50%, at most 60%, at most 70%, at most 75%, at most 80%, at most 85%, at most 90%, at most 95%, at most 99%, or 100%, amino acid sequence identity to a wild type exemplary CRISPR/Cas12d (e.g., SEQ ID NO: 1).

In some cases, the CRISPR/Cas12d can comprise an amino acid sequence having at least 10%, at least 15%, 20%, at least 30%, at least 40%, at least 50%, at least 60%, at least 70%, at least 75%, at least 80%, at least 85%, at least 90%, at least 95%, at least 99%, or 100%, amino acid sequence identity to a wild type exemplary CRISPR/Cas12d (e.g., SEQ ID NO: 1).

In some cases, the CRISPR/Cas12d can comprise an amino acid sequence having at most 10%, at most 15%, at most 20%, at most 30%, at most 40%, at most 50%, at most 60%, at most 70%, at most 75%, at most 80%, at most 85%, at most 90%, at most 95%, at most 99%, or 100%, amino acid sequence identity to the nuclease domain of a wild type exemplary CRISPR/Cas12d (e.g., SEQ ID NO: 1).

The CRISPR/Cas12d proteins disclosed herein may comprise one or more modifications. The modification may comprise a post-translational modification. The modification of the target nucleic acid may occur at least 1 2, 3, 4, 5, 6, 7, 8, 9, 10, 20, 30, 40, 50, 60, 70, 80, 90, 100 or more amino acids away from the either the carboxy terminus or amino terminus end of the CRISPR/Cas12d protein. The modification of the CRISPR/Cas12d protein may occur at most 1, 2, 3, 4, 5, 6, 7, 8, 9, 10, 20, 30, 40, 50, 60, 70, 80, 90, 100 or more amino acids away from the carboxy terminus or amino terminus end of the CRISPR/Cas12d protein. The modification may occur due to the modification of a nucleic acid encoding a CRISPR/Cas12d protein. Exemplary modifications can comprise methylation, demethylation, acetylation, deacetylation, ubiquitination, deubiquitination, deamination, alkylation, depurination, oxidation, pyrimidine dimer formation, transposition, recombination, chain elongation, ligation, glycosylation, phosphorylation, dephosphorylation, adenylation, deadenylation, SUMOylation, deSUMOylation, ribosylation, deribosylation, myristoylation, remodeling, cleavage, oxidoreduction, hydrolation, and isomerization.

The CRISPR/Cas12d can comprise a modified form of a wild type exemplary CRISPR/Cas12d. The modified form of the wild type exemplary CRISPR/Cas12d can comprise an amino acid change (e.g., deletion, insertion, or substitution) that reduces the nucleic acid-cleaving activity of the CRISPR/Cas12d. Alternatively, the amino acid change can result in an increase in nucleic acid-cleaving activity of the CRISPR/Cas12d. Alternatively, the amino acid change can result in a change in the temperature at which the CRISPR/Cas12d is active.

The CRISPR/Cas12d protein may comprise one or more mutations. The CRISPR/Cas12d protein may comprise amino acid modifications (e.g., substitutions, deletions, additions, etc., and combinations thereof). The CRISPR/Cas12d protein may comprise one or more non-native sequences (e.g., a fusion, as defined herein). The amino acid modifications may comprise one or more non-native sequences (e.g., a fusion as defined herein, an affinity tag). The amino acid modifications may not substantially alter the activity of the endonuclease. The CRISPR/Cas12d comprising amino acid modifications and/or fusions may retain at least about 75%, at least about 80%, at least about 85%, at least about 90%, at least about 95%, at least about 97% or 100% activity of the wild-type CRISPR/Cas12d. Modifications (e.g., mutations) of the disclosure can be produced by site-directed mutation. Mutations can include substitutions, additions, and deletions, or any combination thereof. In some instances, the mutation converts the mutated amino acid to alanine. In some instances, the mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagines, glutamine, histidine, lysine, or arginine). The mutation can convert the mutated amino acid to a non-natural amino acid (e.g., selenomethionine). The mutation can convert the mutated amino acid to amino acid mimics (e.g., phospho mimics). The mutation can be a conservative mutation. For example, the mutation can convert the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation).

In some instances, the CRISPR/Cas12d can target nucleic acid. The CRISPR/Cas12d can target DNA. In some instances, the CRISPR/Cas12d is modified to express nickase activity. In some instances, the CRISPR/Cas12d is modified to target nucleic acid but is enzymatically inactive (e.g., does not have endonuclease or nickase activity). In some instances, the CRISPR/Cas12d is modified to express one or more of the following activities, with or without endonuclease activity: nickase, exonuclease, DNA repair (e.g., DNA DSB repair), helicase, transcriptional (co-) activation, transcriptional (co-) repression, methylase, and/or demethylase.

In some instances, the CRISPR/Cas12d is active at temperatures suitable for growth and culture of plants and plant cells, such as for example and not limitation, about 20° C. to about 35° C., preferably about 23° C. to about 32° C., and most preferably about 25° C. to about 28° C. Proof-of-concept experiments can be performed in plant leaf tissue by targeting DSBs to integrated reporter genes and endogenous loci. The technology then can be adapted for use in protoplasts and whole plants, and in viral-based delivery systems. Finally, multiplex genome engineering can be demonstrated by targeting DSBs to multiple sites within the same genome.

The CRISPR/Cas12d can comprise one or more non-native sequences (e.g., a fusion as discussed herein). In some instances, the non-native sequence of the CRISPR/Cas12d comprises a moiety that can alter transcription. Transcription can be increased or decreased. Transcription can be altered by at least about 1-fold, 2-fold, 3-fold, 4-fold, 5-fold, 10-fold, 15-fold, or 20-fold or more. Transcription can be altered by at most about 1-fold, 2-fold, 3-fold, 4-fold, 5-fold, 10-fold, 15-fold, or 20-fold or more. The moiety can be a transcription factor. When a CRISPR/Cas12d is a fusion CRISPR/Cas12d comprising a non-native sequence that can alter transcription, the CRISPR/Cas12d may comprise reduced enzymatic activity as compared to a wild-type CRISPR/Cas12d.

By way of non-limiting example, CRISPR/Cas12d may bind a nucleic acid-targeting nucleic acid (e.g., single-stranded DNA, single-stranded RNA) that guides it to a target nucleic acid that is complementary to the nucleic acid-targeting nucleic acid, wherein the target nucleic acid comprises a dsDNA (e.g., such as a plasmid, genomic DNA, etc.), and thereby carries out site specific cleavage within the target nucleic acid.

In some embodiments of the disclosure, the methods and compositions comprise CRISPR/Cas12d, and said methods and compositions are used at temperatures suitable for growth and culture of certain eukaryotes (e.g., mammals, yeast, fungi, fish, and plants) and eukaryotic (e.g., mammalian, yeast, fungal, fish, and plant) cells, such as for example and not limitation, about 20° C. to about 35° C., preferably about 23° C. to about 32° C., and most preferably about 25° C. to about 28° C.

In some embodiments of the disclosure, the CRISPR/Cas12d is provided separately from the nucleic acid-targeting nucleic acid. In other embodiments, the CRISPR/Cas12d is provided in a complex wherein the nucleic acid-targeting nucleic acid is pre-associated with the CRISPR/Cas12d.

In some embodiments of the disclosure, the CRISPR/Cas12d is provided as part of an expression cassette on a suitable vector, configured for expression of the CRISPR/Cas12d in a desired host cell (e.g., a eukaryotic cell including a plant cell or a plant protoplast). The vector may allow transient expression of the CRISPR/Cas12d. Alternatively, the vector may allow the expression cassette and/or CRISPR/Cas12d to be stably maintained in the host cell, such as for example and not limitation, by integration into the host cell genome, including stable integration into the genome. In some embodiments, the host cell is a progenitor cell, thereby providing heritable expression of the CRISPR/Cas12d. The CRISPR/Cas12d contained in the expression cassette may be a heterologous polypeptide as described below.

In other embodiments, the CRISPR/Cas12d is provided as a heterologous polypeptide, either alone or as a transcriptional or translational fusion (to either or both of the N-terminal and C-terminal domains of the CRISPR/Cas12d), as discussed herein, with one or more functional domains, such as for example and not limitation, a localization signal (e.g., nuclear localization signal, chloroplast localization signal), an epitope tag, an antibody, and/or a functional protein, such as for example and not limitation, a reporter protein (e.g., a fluorescent reporter protein such as mNeonGreen and GFP), proteins involved in DNA break repair (e.g., DNA DSBs), a nickase, a helicase, an exonuclease, a transcriptional (co-) activator, a transcriptional (co-) repressor, a methylase, and/or a demethylase.

Exemplary localization signals may include, but is not limited to, the SV40 nuclear localization signal (Hicks et al., 1993). Other, non-classical types of nuclear localization signal may also be adapted for use with the methods provided herein, such as the acidic M9 domain of hnRNP Al or the PY-NLS motif signal (Dormann et al., 2012). Localization signals also may be incorporated to permit trafficking of the nuclease to other subcellular compartments such as the mitochondria or chloroplasts. Targeting Cas12d components to the chloroplast can be achieved by incorporating in the expression construct a sequence encoding a chloroplast transit peptide (CTP) or plastid transit peptide, operably linked to the 5′ region of the sequence encoding the Cas12d protein.

In other embodiments, the CRISPR/Cas12d is provided as a protein. In still other embodiments, the CRISPR/Cas12d is provided as a nucleic acid, such as for example and not limitation, an mRNA.

In any of the above embodiments, the CRISPR/Cas12d may be optimized for expression in plants, including but not limited to plant-preferred promoters, plant tissue-specific promoters, and/or plant-preferred codon optimization, as discussed in more detail herein. Similar optimization in other eukaryotic organisms is also provided, including use of mammalian-, yeast-, fungal-, or fish-preferred promoters and codon optimization.

In any of the above embodiments, the CRISPR/Cas12d may be present as a fusion (e.g., transcriptional and/or translational fusion) with polynucleotides or polypeptides of interest that are associated with certain plant genes and/or traits. Such plant genes and/or traits include for example and not limitation, an acetolactate synthase (ALS) gene, an enolpyruvylshikimate phosphate synthase gene (EPSPS) gene, a male fertility gene (e.g., MS45, MS26 or MSCA1), a herbicide resistance gene, a male sterility gene, a female fertility gene, a female sterility gene, a male or female restorer gene, and genes associated with the traits of sterility, fertility, herbicide resistance, herbicide tolerance, biotic stress such as fungal resistance, viral resistance, or insect resistance, abiotic stress such as drought tolerance, chilling tolerance, or cold tolerance, nitrogen use efficiency, phosphorus use efficiency, water use efficiency and crop or biomass yield (e.g., improved or decreased crop or biomass yield), and mutants of such genes. Such mutants include, for example and not limitation, amino acid substitutions, deletions, insertions, codon optimization, and regulatory sequence changes to alter the gene expression profiles.

Nucleic Acid-Targeting Nucleic Acids (Nucleic Acid-Targeting Guide Nucleic Acids) are also provided.

Disclosed herein are nucleic acid-targeting nucleic acids (nucleic acid-targeting guide nucleic acids) that can direct the activities of an associated polypeptide (e.g., CRISPR/Cas12d protein, including one of SEQ ID NO: 1) to a specific target sequence within a target nucleic acid. The nucleic acid-targeting nucleic acid can comprise nucleotides. The nucleic acid-targeting nucleic acid may be a single-stranded RNA (ssRNA). In certain embodiments, nucleic acid-targeting nucleic acids can be located in Clustered Regularly Interspersed Short Palindromic Repeats (CRISPR) RNAs (crRNAs) or comprise the spacer elements in chimeric cr/scoutRNA hybrid (sgRNAs) provided herein.

A nucleic acid-targeting nucleic acid can comprise one or more modifications (e.g., a base modification, a backbone modification), to provide the nucleic acid with a new or enhanced feature (e.g., improved stability). The one or more modifications may, in addition to or independently of improving stability, change the binding specificity of the nucleic acid-targeting nucleic acid in a user-preferred way (e.g., greater or lesser specificity or tolerance or lack of tolerance for a specific mismatch). The one or more modifications, whether to improve stability or alter binding specificity or both, preserve the ability of the nucleic acid-targeting nucleic acid to interact with both CRISPR/Cas12d and the target nucleic acid. A nucleic acid-targeting nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acid-targeting nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acid-targeting nucleic acids, the phosphate groups can commonly be referred to as forming the internucleoside backbone of the nucleic acid-targeting nucleic acid. The linkage or backbone of the nucleic acid-targeting nucleic acid can be a 3′ to 5′ phosphodiester linkage.

The nucleic acid-targeting nucleic acid can be a ssRNA. In a preferred embodiment, the nucleic acid-targeting nucleic acid is a short ssRNA. In some embodiments, the ssRNA is 50 nucleotides or less in length, preferably 40 nucleotides or less in length, and most preferably 30 nucleotides or less in length. In a particularly preferred embodiment, the nucleic acid-targeting nucleic acid is a 5′-phosphorylated ssRNA of 20, 21 22, 23, 24, 25, 26, 27, 28, 29, or 30 nucleotides in length.

Modified backbones can include those that retain a phosphorus atom in the backbone and those that do not have a phosphorus atom in the backbone. Suitable modified nucleic acid-targeting nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable nucleic acid-targeting nucleic acids having inverted polarity can comprise a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included. A nucleic acid-targeting nucleic acid can comprise one or more phosphorothioate and/or heteroatom internucleoside linkages. A nucleic acid-targeting nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage. A nucleic acid-targeting nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

A nucleic acid-targeting nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid-targeting nucleic acid can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acid-targeting nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT (dimethoxytrityl) protected phosphoramidite monomers can be prepared and used for oligomeric compound synthesis using phosphoramidite chemistry. The incorporation of CeNA monomers into a nucleic acid chain can increase the stability of a DNA RNA hybrid. CeNA oligoadenylates can form complexes with nucleic acid complements with similar stability to the native complexes. A further modification can include LNAs in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (˜CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid-targeting nucleic acid can comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted Ci to Cio alkyl or C2 to C10 alkenyl and alkynyl. Particularly suitable are O((CH2)nO)mCH3, O(CH2) OCH3, O(CH2)nNH2, O(CH2) CH3, O(CH2) ONH2, and O(CH2) ON((CH2) CH3)2, where n and m are from 1 to about 10. A sugar substituent group can be selected from: CI to CIO lower alkyl, substituted lower alkyl, alkenyl, alkynyl, alkaryl, aralkyl, O-alkaryl or O-aralkyl, SH, SCH3, OCN, CI, Br, CN, CF3, OCF3, SOCFfa, SO2CH3, ON02, N02, N3, NH2, heterocycloalkyl, heterocycloalkaryl, amino alkylamino, poly alky lamino, substituted silyl, an RNA cleaving group, a reporter group, an intercalator, a group for improving the pharmacokinetic properties of a nucleic acid-targeting nucleic acid, or a group for improving the pharmacodynamic properties of a nucleic acid-targeting nucleic acid, and other substituents having similar properties. A suitable modification can include 2′-methoxyethoxy (2′-O—CH2CH2OCH3, also known as 2′-O-(2-methoxyethyl) or 2′-MOE i.e., an alkoxyalkoxy group). A further suitable modification can include 2′-dimethylaminooxyethoxy, (i.e., a O(CH2)2ON(CH3)2 group, also known as 2′-DMAOE), and 2′-dimethylaminoethoxyethoxy (also known as 2′-O-dimethyl-amino-ethoxy-ethyl or 2′-DMAEOE), i.e., 2′-O-CH2-O-CH2-N(CH3)2. Other suitable sugar substituent groups can include methoxy (—O—CH3), aminopropoxy (-0 CH2CH2CH2NH2), allyl (—CH2-CH═C—), —O-allyl (—O— CH2-CH═CH2) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

A nucleic acid-targeting nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (˜C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(14)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (14)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and -2, N-6 and 0-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of a nucleic acid-targeting nucleic acid can comprise chemically linking to the nucleic acid-targeting nucleic acid one or more moieties or conjugates that can enhance the activity, cellular distribution or cellular uptake of the nucleic acid-targeting nucleic acid. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety. A modification may also include a “Protein Transduction Domain” or PTD (i.e., a cell penetrating peptide (CPP)). The PTD can refer to a polypeptide, polynucleotide, carbohydrate, or organic or inorganic compound that facilitates traversing a lipid bilayer, micelle, cell membrane, organelle membrane, or vesicle membrane. A PTD can be attached to another molecule, which can range from a small polar molecule to a large macromolecule and/or a nanoparticle, and can facilitate the molecule traversing a membrane, for example going from extracellular space to intracellular space, or cytosol to within an organelle. Various types of nanoparticles may be used, as described in WO2008/043156, US 20130185823, and WO2015089419. A PTD can be covalently linked to the amino terminus of a polypeptide. A PTD can be covalently linked to the carboxyl terminus of a polypeptide. A PTD can be covalently linked to a nucleic acid. Exemplary PTDs can include, but are not limited to, a minimal peptide protein transduction domain; a polyarginine sequence comprising a number of arginines sufficient to direct entry into a cell (e.g., 3, 4, 5, 6, 7, 8, 9, 10, or 10-50 arginines), a VP22 domain, polylysine, and transportan, arginine homopolymer of from 3 arginine residues to 50 arginine residues. The PTD can be an activatable CPP (ACPP). ACPPs can comprise a polycationic CPP (e.g., Arg9 or “R9”) connected via a cleavable linker to a matching polyanion (e.g., Glu9 or ‘E9”), which can reduce the net charge to nearly zero and thereby inhibits adhesion and uptake into cells. Upon cleavage of the linker, the polyanion can be released, locally unmasking the polyarginine and its inherent adhesiveness, thus “activating” the ACPP to traverse the membrane.

Still other modifications of a nucleic-acid targeting nucleic acid can comprise a 5′ cap, a 3′ polyadenylated tail, a riboswitch sequence, a stability control sequence, a sequence that forms a dsRNA duplex, a modification or sequence that targets the nucleic-acid targeting nucleic acid to a subcellular location, a modification or sequence that provides for tracking a modification or sequence that provides a binding site for proteins, a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer molecule, a 5′ to 3′ covalent linkage, or any combination thereof.

The nucleic acid-targeting nucleic acid can be at least about 5, 6, 7, 8, 9, 10, 11 12, 13, 14, 15, 16, 17, 18, 19, 20, 21, 22, 23, 24, 25, 26, 27, 28, 29, or 30 or more nucleotides in length. The nucleic acid-targeting nucleic acid can be at most about 5, 6, 7, 8, 9, 10, 11, 12, 13, 14, 15, 16, 17, 18, 19, 20, 21, 22, 23, 24, 25, 26, 27, 28, 29, or 30 or more nucleotides in length. In some instances, the nucleic acid-targeting nucleic acid is 20, 21 22, 23, 24, 25, 26, 27, 28, 29, or 30 nucleotides in length. In some instances, the nucleic acid-targeting nucleic acid is phosphorylated at either the 5′ or 3′ end, or both ends.

The nucleic acid-targeting nucleic acid can comprise a 5′ deoxycytosine. The nucleic acid-targeting nucleic acid can comprise a deoxycytosine-deoxyadenosine at the 5′ end of the nucleic acid-targeting nucleic acid. In some embodiments, any nucleotide can be present at the 5′ end, and/or can contain a modified backbone or other modifications as discussed herein. The nucleic acid-targeting nucleic acid may comprise a 5′ phosphorylated end.

The nucleic acid-targeting nucleic acid can be fully complementary to the target nucleic acid (e.g., hybridizable). The nucleic acid-targeting nucleic acid can be partially complementary to the target nucleic acid. For example, the nucleic acid-targeting nucleic acid can be at least 30, 40, 50, 60, 70, 80, 90, 95, or 100% complementary to the target nucleic acid over the region of the nucleic acid-targeting nucleic acid. The nucleic acid-targeting nucleic acid can be at most 30, 40, 50, 60, 70, 80, 90, 95, or 100% complementary to the target nucleic acid over the region of the nucleic acid-targeting nucleic acid.

A stretch of nucleotides of the nucleic acid-targeting nucleic acid can be complementary to the target nucleic acid (e.g., hybridizable). A stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 contiguous nucleotides can be complementary to target nucleic acid. A stretch of at most 2, 3, 4, 5, 6, 7, 8, 9, 10, 11, 12, 13, 14, 15, 16, 17, 18, 19, 20, 21, 22, 23, 24, 25, 26, 27, 28, 29, or 30 contiguous nucleotides can be complementary to target nucleic acid.

A portion of the nucleic acid-targeting nucleic acid which is fully complementary to the target nucleic acid may extend from at least nucleotide 2, to nucleotide 17 (as counted from the 5′ end of the nucleic acid-targeting nucleic acid). A portion of the nucleic acid-targeting nucleic acid which is fully complementary to the target nucleic acid may extend from at least nucleotide 3 to nucleotide 20, nucleotide 4 to nucleotide 18, nucleotide 5 to nucleotide 16, nucleotide 6 to nucleotide 14, nucleotide 7 to nucleotide 12, nucleotide 6 to nucleotide 16, nucleotide 6 to nucleotide 18, or nucleotide 6 to nucleotide 20.

The nucleic acid-targeting nucleic acid can hybridize to a target nucleic acid. The nucleic acid-targeting nucleic acid can hybridize with a mismatch between the nucleic acid-targeting nucleic acid and the target nucleic acid (e.g., a nucleotide in the nucleic acid-targeting nucleic acid may not hybridize with the target nucleic acid). A nucleic acid-targeting nucleic acid can comprise at least 1 2, 3, 4, 5, 6, 7, 8, 9, or 10 or more mismatches when hybridized to a target nucleic acid. A nucleic acid-targeting nucleic acid can comprise at most 1, 2, 3, 4, 5, 6, 7, 8, 9, or 10 or more mismatches when hybridized to a target nucleic acid.

The nucleic acid-targeting nucleic acid may direct cleavage of the target nucleic acid at the bond between the 1st and 2nd, 2nd and 3rd, 3rd and 4th, 4th and 5th, 5th and 6th, 6th and 7th, 7th and 8th, 8th and 9th, 9th and 10th, 10th and 11th, 11th and 12th, 12th and 13th, 13th and 14th, 14th and 15th, 15th and 16th, 16th and 17th, 17th and 18th, 18th and 19th, 19th and 20th, 20th and 21st, 21st and 22nd, 22nd and 23rd, 23rd and 24th, or 24th and 25th nucleotides relative to the 5′-end of the designed nucleic acid-targeting nucleic acid. The designed nucleic acid-targeting nucleic acid may direct cleavage of the target nucleic acid at the bond between the 10th and 11th nucleotides (tlO and tl1) relative to the 5′-end of the designed nucleic acid-targeting nucleic acid. The precise design for optimum cleavage of the target nucleic acid cleavage site may be determined by preliminary tests with plasmid targets incorporating the cleavage site.

As discussed herein, the nucleic acid-targeting nucleic acid can be a ssRNA. In a preferred embodiment, the nucleic acid-targeting nucleic acid is a short ssRNA. In some embodiments, the ssRNA is 50 nucleotides or less in length, preferably 40 nucleotides or less in length, most preferably 30 nucleotides or less in length. In a particularly preferred embodiment, the nucleic acid-targeting nucleic acid is 20, 21 22, 23, 24, 25, 26, 27, 28, 29, or 30 nucleotides in length.

### Target Nucleic Acids

The target nucleic acid may comprise one or more sequences that are at least partially complementary to one or more designed nucleic acid-targeting nucleic acids. The target nucleic acid can be part or all of a gene, a 5′ end of a gene, a 3′ end of a gene, a regulatory element (e.g., promoter, enhancer), a pseudogene, non-coding DNA, a microsatellite, an intron, an exon, chromosomal DNA, mitochondrial DNA, sense DNA, antisense DNA, nucleoid DNA, chloroplast DNA, or RNA among other nucleic acid entities. The target nucleic acid can be part or all of a plasmid DNA. The plasmid DNA or a portion thereof may be negatively supercoiled. The target nucleic acid can be in vitro or in vivo.

The target nucleic acid may comprise a sequence within a low GC content region. The target nucleic acid may be negatively supercoiled. Thus, by non-limiting example, the target nucleic acid may comprise a GC content of at least about 5, 10, 15, 20, 25, 30, 35, 40, 45, 50, 55, 60, or 65% or more. The target nucleic acid may comprise a GC content of at most about 5, 10, 15, 20, 25, 30, 35, 40, 45, 50, 55, 60, or 65% or more.

A region comprising a particular GC content may be the length of the target nucleic acid that hybridizes with the designed nucleic acid-targeting nucleic acid. The region comprising the GC content may be longer or shorter than the length of the region that hybridizes with the designed nucleic acid-targeting nucleic acid. The region comprising the GC content may be at least 30, 40, 50, 60, 70, 80, 90 or 100 or more nucleotides longer or shorter than the length of the region that hybridizes with the designed nucleic acid-targeting nucleic acid. The region comprising the GC content may be at most 30, 40, 50, 60, 70, 80, 90 or 100 or more nucleotides longer or shorter than the length of the region that hybridizes with the designed nucleic acid-targeting nucleic acid.

The DNA targeted by an individual spacer element in the crRNA or sgRNA comprises a protospacer associated motif (PAM) made up of a TA or TG dinucleotide located immediately upstream (i.e., 5′) of the DNA equivalent sequence of the spacer element.

In some embodiments, the target nucleic acid is found within a plant genome. The plant can be a monocot or a dicot. Non-limiting examples of monocots include maize, rice, sorghum, rye, barley, wheat, millet, oats, sugarcane, turfgrass, or switchgrass. Non-limiting examples of dicots include soybean, canola, alfalfa, sunflower, cotton, tobacco, peanut, potato, winter oil seed rape, spring oil seed rape, sugar beet, fodder beet, red beet, sunflower, tobacco, Arabidopsis, or safflower. In some embodiments, the target nucleic acid comprises an acetolactate synthase (ALS) gene (including mutants thereof), an Enolpyruvylshikimate Phosphate Synthase Gene (EPSPS) gene (including mutants of the EPSPS gene such as for example and not limitation T102I/P106A, T102I/P106S, T102I/P106C, G101A/A192T, and G101A/A144D), a male fertility (MS45, MS26 or MSCA1) gene (including mutants thereof), a male sterility gene, a sterility restorer gene, a herbicide resistance gene, a herbicide tolerance gene, a fungal resistance gene, a viral resistance gene, an insect resistance gene, a gene associated with increased or decreased plant yield (e.g., biomass or seeds), a gene associated with drought, chilling or cold resistance/tolerance, with nitrogen, phosphorus or water use efficiency, or another target site described in WO2015/026883. The target nucleic acid may include genes associated with one or more of the following traits: herbicide resistance, herbicide tolerance, biotic stress resistance, fungal resistance, viral resistance, insect resistance, increased or decreased plant yield (e.g., biomass or seeds), abiotic stress resistance, nitrogen use efficiency, phosphorus use efficiency, water use efficiency, and drought resistance. In some embodiments, the target nucleic acid is found within the genome of another eukaryotic cell including a mammal, yeast, fungal, or fish cell. The target nucleic acid may include mutations such as for example and not limitation, amino acid substitutions, deletions, insertions, codon optimization, and regulatory sequence changes to alter the gene expression profiles. The target nucleic acid may further include any of the nucleic acids for use with the disclosure as described hereinbelow.

Any nucleic acid of interest can be provided, integrated into the host cell genome (e.g., a plant cell or protoplast) at the target nucleic acid or transiently maintained within the host cell, and expressed in the host cell by using the invented methods and compositions. Such nucleic acid may be non-native. The nucleic acid of interest may include mutations such as for example and not limitation, amino acid substitutions, deletions, insertions, regulatory sequence changes to alter the gene expression profiles, transcriptional and/or translational fusions as discussed herein, and/or codon optimization. One or more nucleic acids of interest may be used in the methods and compositions described herein. The one or more nucleic acids may be present as a fusion (e.g., transcriptional and/or translational fusion) with CRISPR/Cas12d.

Nucleic acids/polypeptides of interest include, but are not limited to, herbicide-resistance coding sequences, herbicide-tolerance coding sequences, insecticidal/insect resistance coding sequences, nematocidal coding sequences, antimicrobial coding sequences, antifungal/fungal resistance coding sequences, antiviral/viral resistance coding sequences (including both RNA and DNA viruses), abiotic and biotic stress tolerance coding sequences, or sequences modifying plant traits such as yield, grain quality, nutrient content, starch quality and quantity, nitrogen fixation and/or utilization, fatty acids, and oil content and/or composition.

Other polynucleotides of interest include sterility and/or fertility genes, such as for example and not limitation, male sterility and male fertility genes. More specific polynucleotides of interest include, but are not limited to, genes that improve crop yield, genes that decrease crop yield, polynucleotides that improve desirability of crops, genes encoding proteins conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms, and genes conferring herbicide tolerance. General categories of genes of interest include, for example, those genes involved in information, such as zinc fingers, those involved in communication, such as kinases, and those involved in housekeeping, such as heat shock proteins.

Examples of genes involved in abiotic stress tolerance include transgenes capable of reducing the expression and/or the activity of poly(ADP-ribose) polymerase (PARP) gene in the plant cells or plants as described in WO 00/04173 or, WO/2006/045633; transgenes capable of reducing the expression and/or the activity of the PARG encoding genes of the plants or plants cells, as described e.g. in WO 2004/090140; and transgenes coding for a plant-functional enzyme of the nicotinamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphoribosyl transferase, enzymes involved in carbohydrate biosynthesis, enzymes involved in the production of polyfructose, especially of the inulin and levan-type.

Examples of genes that improve drought resistance are described, for example, in WO 2013122472. The absence or reduced level of functional Ubiquitin Protein Ligase protein (UPL)protein, more specifically, UPL3, can decrease need for water or otherwise improve resistance to drought of said plant. Other examples of transgenic plants with increased drought tolerance are disclosed in, for example, US 2009/0144850, US 2007/0266453, and WO 2002/083911. US2009/0144850 describes a plant displaying a drought tolerance phenotype due to altered expression of a DR02 nucleic acid. US 2007/0266453 describes a plant displaying a drought tolerance phenotype due to altered expression of a DR03 nucleic acid and WO 2002/083911 describes a plant having an increased tolerance to drought stress due to a reduced activity of an ABC transporter which is expressed in guard cells. Overexpression of DREB1A in transgenic plants can activate the expression of many stress tolerance genes under normal growing conditions and resulted in improved tolerance to drought, salt loading, and freezing.

More specific categories of transgenes, for example, include genes encoding important traits for agronomics, insect resistance, disease resistance, herbicide resistance, fertility or sterility, grain characteristics, and commercial products. Genes of interest include, generally, those involved in oil, starch, carbohydrate, or nutrient metabolism as well as those affecting kernel size, sucrose loading, and the like that can be stacked or used in combination with other traits, such as but not limited to herbicide resistance, described herein. The polypeptide encoded by any of the foregoing polynucleotides may also be used in the methods and compositions herein, such as for example and not limitation, incorporation into a host cell (e.g., a plant cell or protoplast), in a fusion with CRISPR/Cas12d and/or in an expression cassette with CRISPR/Cas12d. One or more polypeptides may be present in said method or composition.

Agronomically important traits such as oil, saccharose, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049; 5,885,801; 5,885,802; and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. Eur. J. Biochem. (1987) 165:99-106, the disclosures of which are herein incorporated by reference.

Commercial traits can also be encoded on a polynucleotide of interest that could increase for example, starch or saccharose for ethanol production, or provide expression of proteins. Another important commercial use of transformed plants is the production of polymers and bioplastics such as described in U.S. Pat. No. 5,602,321. Genes such as (3-Ketothiolase, PHBase (polyhydroxybutyrate synthase), and acetoacetyl-CoA reductase (see Schubert et al., J. Bacteriol. (1988) 170:5837-5847) facilitate expression of polyhydroxyalkanoates (PHAs).

The Cas12d system and methods described herein can be used to introduce targeted double-strand breaks (DSB) in an endogenous DNA sequence. The DSB activates cellular DNA repair pathways, which can be harnessed to achieve desired DNA sequence modifications near the break site. This is of interest where the inactivation of endogenous genes can confer or contribute to a desired trait. In particular embodiments, homologous recombination with a template sequence is promoted at the site of the DSB, in order to introduce a gene of interest.

In particular embodiments, non-transgenic genetically modified plants, plant parts or cells are obtained, in that no exogenous DNA sequence is incorporated into the genome of any of the plant cells of the plant. Where only the modification of an endogenous gene is ensured and no foreign genes are introduced or maintained in the plant genome; the resulting genetically modified crops contain no foreign genes and can thus basically be considered non-transgenic.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Illinois), pp. 497-502; herein incorporated by reference); corn (Pedersen et al, J. Biol. Chem. (1986) 261:6279; Kirihara et al, Gene (1988) 71:359; both of which are herein incorporated by reference); and rice (Musumura et al., Plant Mol. Biol. (1989) 12: 123, herein incorporated by reference). Other agronomically important genes encode latex, Floury 2, growth factors, seed storage factors, and transcription factors.

Polynucleotides that improve crop yield include dwarfing genes, such as Rht1 and Rht2 (Peng et al., Nature (1999) 400:256-261), and those that increase plant growth, such as ammonium-inducible glutamate dehydrogenase. Polynucleotides that improve desirability of crops include, for example, those that allow plants to have reduced saturated fat content, those that boost the nutritional value of plants, and those that increase grain protein. Polynucleotides that improve salt tolerance are those that increase or allow plant growth in an environment of higher salinity than the native environment of the plant into which the salt-tolerant gene(s) has been introduced.

Polynucleotides/polypeptides that influence amino acid biosynthesis include, for example, anthranilate synthase (AS; EC 4.1.3.27) which catalyzes the first reaction branching from the aromatic amino acid pathway to the biosynthesis of tryptophan in plants, fungi, and bacteria. In plants, the chemical processes for the biosynthesis of tryptophan are compartmentalized in the chloroplast. See, for example, US Pub. 2008/0050506, herein incorporated by reference. Additional sequences of interest include Chorismate Pyruvate Lyase (CPL) which refers to a gene encoding an enzyme which catalyzes the conversion of chorismate to pyruvate and pHBA. The most well characterized CPL gene has been isolated from E. coli and bears the GenBank accession number M96268. See, U.S. Pat. No. 7,361,811, herein incorporated by reference.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al, Science (1994) 266:789; Martin et al, Science (1993) 262: 1432; and Mindrinos et al, Cell (1994) 78: 1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al, Gene (1986) 48: 109); and the like.

A plant can be transformed with cloned resistance genes to engineer plants that are resistant to specific pathogen strains. See, e.g., Jones et al., Science 266:789 (1994) (cloning of the tomato Cf-9 gene for resistance to Cladosporium fulvum); Martin et al., Science 262: 1432 (1993) (tomato Pto gene for resistance to Pseudomonas syringae pv. tomato encodes a protein kinase); Mindrinos et al., Cell 78: 1089 (1994). A plant can be transformed with cloned resistance genes conferring resistance to a pest, such as soybean cyst nematode. See e.g., PCT Application WO 96/30517 and PCT Application WO 93/19181. A plant can be transformed with genes encoding Bacillus thuringiensis proteins. See, e.g., Geiser et al., Gene 48: 109 (1986). A plant can be transformed with genes involved in production of lectins. See, for example, Van Damme et al., Plant Molec. Biol. 24:25 (1994).

A plant can be transformed with genes encoding vitamin-binding protein, such as avidin. See, PCT application US93/06487, describing the use of avidin and avidin homologues as larvicides against insect pests. A plant can be transformed with genes encoding enzyme inhibitors such as protease or proteinase inhibitors or amylase inhibitors. See, e.g., Abe et al., J. Biol. Chem. 262: 16793 (1987), Huub et al, Plant Molec. Biol. 21:985 (1993); Sumitani et al, Biosci. Biotech. Biochem. 57: 1243 (1993) and U.S. Pat. No. 5,494,813. A plant can be transformed with genes encoding insect-specific hormones or pheromones such as ecdysteroid or juvenile hormone, a variant thereof, a mimetic based thereon, or an antagonist or agonist thereof. See, e.g., Hammock et al., Nature 344:458 (1990).

A plant can be transformed with genes encoding insect-specific peptides or neuropeptides which, upon expression, disrupts the physiology of the affected pest. See, e.g., Regan, J. Biol. Chem. 269:9 (1994) and Pratt et al, Biochem. Biophys. Res. Comm. 163: 1243 (1989). See also U.S. Pat. No. 5,266,317. A plant can be transformed with genes encoding proteins and peptides that are part of insect-specific venom produced in nature by a snake, a wasp, or any other organism. For example, see Pang et al., Gene 1 16: 165 (1992). A plant can be transformed with genes encoding enzymes responsible for a hyperaccumulation of a monoterpene, a sesquiterpene, a steroid, hydroxamic acid, a phenylpropanoid derivative or another nonprotein molecule with insecticidal activity. A plant can be transformed with genes encoding enzymes involved in the modification, including the post-translational modification, of a biologically active molecule; for example, a glycolytic enzyme; a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO93/02197, Kramer et al, Insect Biochem. Molec. Biol. 23:691 (1993) and Kawalleck et al, Plant Molec. Biol. 21:673 (1993).

A plant can be transformed with genes encoding molecules that stimulate signal transduction. For example, see Botella et al., Plant Molec. Biol. 24:757 (1994), and Griess et al., Plant Physiol. 104: 1467 (1994). A plant can be transformed with genes encoding viral-invasive proteins or a complex toxin derived therefrom. See Beachy et al., Ann. rev. Phytopathol. 28:451 (1990). A plant can be transformed with genes encoding developmental-arrestive proteins produced in nature by a pathogen or a parasite. See Lamb et al., Bio/Technology 10: 1436 (1992) and Toubart et at, Plant J. 2:367 (1992). A plant can be transformed with genes encoding a developmental-arrestive protein produced in nature by a plant. For example, Logemann et al., Bio/Technology 10:305 (1992).

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS), in particular the sulfonyl urea-type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g., the HPPD gene) or other such genes known in the art. See, for example, U.S. Pat. Nos. 7,626,077; 5,310,667; 5,866,775; 6,225,114; 6,248,876; 7,169,970; and 6,867,293. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling, particularly of maize. Examples of genes used in such ways include male fertility genes such as MS26 (see for example U.S. Pat. Nos. 7,098,388; 7,517,975; and 7,612,251), MS45 (see for example U.S. Pat. Nos. 5,478,369 and 6,265,640) or MSCA1 (see for example U.S. Pat. No. 7,919,676). Other genes include kinases and those encoding compounds toxic to either male or female gametophytic development.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA.

Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference in their entireties.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), yellow-green fluorescent protein (mNeonGreen) and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification. Additional selectable markers include genes that confer resistance to herbicidal compounds, such as glufosinate ammonium, bromoxynil, imidazolinones, and 2, 4-dichlorophenoxy acetate (2,4-D). See for example, Yarranton, Curr Opin Biotech (1992) 3:506-11; Christopherson et al., Proc. Natl. Acad. Sci. USA (1992) 89:6314-8; Yao et al, Cell (1992) 71:63-72; Reznikoff, Mol Microbiol (1992) 6:2419-22; Hu et al, Cell (1987) 48:555-66; Brown et al, Cell (1987) 49:603-12; Figge et al, Cell (1988) 52:713-22; Deuschle et al, Proc. Natl. Acad. Sci. USA (1989) 86:5400-4; Fuerst et al, Proc. Natl. Acad. Sci. USA (1989) 86:2549-53; Deuschle et al, Science (1990) 248:480-3; Gossen, Ph.D. Thesis, University of Heidelberg (1993); Reines et al., Proc. Natl. Acad. Sci. USA (1993) 90: 1917-21; Labow et al, Mol Cell Biol (1990) 10:3343-56; Zambretti et al, Proc. Natl. Acad. Sci. USA (1992) 89:3952-6; Bairn et al, Proc. Natl. Acad. Sci. USA (1991) 88:5072-6; Wyborski et al, Nucleic Acids Res (1991) 19:4647-53; Hillen and Wissman, Topics Mol Struc Biol (1989) 10: 143-62; Degenkolb et al, Antimicrob Agents Chemother (1991) 35: 1591-5; Kleinschnidt et al, Biochemistry (1988) 27: 1094-104; Bonin, Ph.D. Thesis, University of Heidelberg (1993); Gossen et al, Proc. Natl. Acad. Sci. USA (1992) 89:5547-51; Oliva et al, Antimicrob Agents Chemother (1992) 36:913-9; Hlavka et al., Handbook of Experimental Pharmacology (1985), Vol. 78 (Springer-Verlag, Berlin); Gill et al, Nature (1988) 334:721-4.

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content. The transgenes, recombinant DNA molecules, DNA sequences of interest, and polynucleotides of interest can comprise one or more DNA sequences for gene silencing. Methods for gene silencing involving the expression of DNA sequences in plant are known in the art include, but are not limited to, cosuppression, antisense suppression, double-stranded RNA (dsRNA) interference, hairpin RNA (hpRNA) interference, intron-containing hairpin RNA (ihpRNA) interference, transcriptional gene silencing, and micro RNA (miRNA) interference.

In some embodiments, the nucleic acid must be optimized for expression in plants. As used herein, a “plant-optimized nucleotide sequence” is a nucleotide sequence that has been optimized for increased expression in plants, particularly for increased expression in plants or in one or more plants of interest. For example, a plant-optimized nucleotide sequence can be synthesized by modifying a nucleotide sequence encoding a protein such as, for example, double-strand-break-inducing agent (e.g., an endonuclease) as disclosed herein, using one or more plant-preferred codons for improved expression. See, for example, Campbell and Gowri, Plant Physiol. (1990) 92: 1-1 1 for a discussion of host-preferred codon usage.

Methods are available in the art for synthesizing plant-preferred genes. See, for example, U.S. Pat. Nos. 5,380,831, and 5,436,391 and Murray et al, Nucleic Acids Res. (1989) 17:477-498, herein incorporated by reference. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

Transformation Methods for Use with the Disclosure

A variety of methods are known for the introduction of nucleotide sequences and polypeptides into an organism, including, for example, transformation, sexual crossing, and the introduction of the polypeptide, DNA, or mRNA into the cell.

In some embodiments, the disclosure comprises breeding of plants comprising one or more transgenic traits. Most commonly, transgenic traits are randomly inserted throughout the plant genome as a consequence of bacterial transformation systems, such as for example and not limitation, those based on Agrobacterium, biolistics, grafting, insect vectors, DNA abrasion, or other commonly used procedures. More recently, gene targeting protocols have been developed that enable directed transgene insertion. One important technology, site-specific integration (SSI) enables the targeting of a transgene to the same chromosomal location as a previously inserted transgene. Custom-designed meganucleases and custom-designed zinc finger meganucleases allow researchers to design nucleases to target specific chromosomal locations, and these reagents allow the targeting of transgenes at the chromosomal site cleaved by these nucleases.

The currently used systems for precision genetic engineering of eukaryotic genomes, e.g., plant genomes, rely upon homing endonucleases, meganucleases, zinc finger nucleases, and transcription activator-like effector nucleases (TALENs), which require de novo protein engineering for every new target locus. The highly specific, CRISPR/Cas12d endonuclease system described herein, is more easily customizable and therefore more useful when modification of many different target sequences is the goal.

Transformation methods in plants may include direct and indirect methods of transformation. Delivery into plant cells by any of the above methods may further include use of one or more cell-penetrating peptides (CPPs). Cells suitable for transformation include, for example and not limitation, plastids and protoplasts.

Suitable direct transformation methods include, for example and not limitation, PEG-induced DNA uptake, pollen tube mediated introduction directly into fertilized embryos/zygotes, liposome-mediated transformation, biolistic methods, by means of particle bombardment, electroporation or microinjection. Indirect methods include, for example and not limitation, bacteria-mediated transformation, (e.g., the Agrobacterium-mediated transformation technology) or viral infection using viral vectors. In the case of biolistic transformation, the nuclease can be introduced into plant tissues with a biolistic device that accelerates the microprojectiles to speeds of 300 to 600 m/s to penetrate plant cell walls and membranes. Another method for introducing protein or RNA to plants is via the sonication of target cells. Liposome or spheroplast fusion may also be used to introduce exogenous material into plants. Electroporation may be used to introduce exogenous material into protoplasts, whole cells and tissues.

Exemplary viral vector include, but are not limited to, a vector from a DNA virus such as, without limitation, geminivirus, cabbage leaf curl virus, bean yellow dwarf virus, wheat dwarf virus, tomato leaf curl virus, maize streak virus, tobacco leaf curl virus, tomato golden mosaic virus, or Faba bean necrotic yellow virus, or a vector from an RNA virus such as, without limitation, a tobravirus (e.g., tobacco rattle virus, tobacco mosaic virus), potato virus X, or barley stripe mosaic virus.

Also, shuttle vectors or binary vectors can be stably integrated into the plant genome, for example via Agrobacterium-mediated transformation. The CRISPR/Cas12d transgene can then be removed by genetic cross and segregation, for production of non-transgenic, but genetically modified plants or crops. In the case of Agrobacterium-mediated transformation, a marker cassette may be adjacent to or between flanking T-DNA borders and contained within a binary vector. In another embodiment, the marker cassette may be outside of the T-DNA. A selectable marker cassette may also be within or adjacent to the same T-DNA borders as the expression cassette or may be somewhere else within a second T-DNA on the binary vector (e.g., a 2 T-DNA system).

The methods and compositions disclosed herein can be used to insert exogenous sequences into a predetermined location in a plant cell genome. Accordingly, genes encoding, e.g., pathogen resistance proteins, enzymes of metabolic pathways, receptors or transcription factors can be inserted, by targeted recombination, into regions of a plant genome favorable to their expression.

Methods for contacting, providing, and/or introducing a composition into various organisms are known and include but are not limited to, stable transformation methods, transient transformation methods, virus-mediated methods, and sexual breeding. Stable transformation indicates that the introduced polynucleotide integrates into the genome of the organism and is capable of being inherited by progeny thereof. Transient transformation indicates that the introduced composition is only temporarily expressed or present in the organism. Protocols for introducing polynucleotides and polypeptides into plants may vary depending on the type of plant or plant cell targeted for transformation, such as monocot or dicot. Suitable methods of introducing polynucleotides and polypeptides into plant cells and subsequent insertion into the plant genome include (in addition to those listed herein) polyethylene glycol-mediated transformation, microparticle bombardment, pollen-tube mediated introduction into fertilized embryos/zygotes, microinjection (Crossway et al., Biotechniques (1986) 4:320-34 and U.S. Pat. No. 6,300,543), meristem transformation (U.S. Pat. No. 5,736,369), electroporation (Riggs et al., Proc. Natl. Acad. Sci. USA (1986) 83:5602-6), Agrobacterium-mediated transformation (U.S. Pat. Nos. 5,563,055 and 5,981,840), direct gene transfer (Paszkowski et al, EMBO J. (1984) 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al., Biotechnology (1988) 6:923-6; Weissinger et al., Ann Rev Genet (1988) 22:421-77; Sanford et al., Particulate Science and Technology (1987) 5:27-37 (onion); Christou et al, Plant Physiol (1988) 87:67-74 (soybean); Finer and McMullen, In Vitro Cell Dev Biol (1991) 27P: 175-82 (soybean); Singh et al, Theor Appl Genet (1998) 96:319-24 (soybean); Datta et al, Biotechnology (1990) 8:736-40 (rice); Klein et al, Proc. Natl. Acad. Sci. USA (1988) 85:4305-9 (maize); Klein et al, Biotechnology (1988) 6:559-63 (maize); U.S. Pat. Nos. 5,240,855; 5,322,783 and 5,324,646; Klein et al, Plant Physiol (1988) 91:440-4 (maize); Fromm et al., Biotechnology (1990) 8:833-9 (maize); Hooykaas-Van Slogteren et al., Nature (1984) 31 1:763-4; U.S. Pat. No. 5,736,369 (cereals); Bytebier et al, Proc. Natl. Acad. Sci. USA (1987) 84:5345-9 (Liliaceae); De Wet et al, (1985) in The Experimental Manipulation of Ovule Tissues, ed. Chapman et al., (Longman, New York), pp. 197-209 (pollen); Kaeppler et al, Plant Cell Rep (1990) 9:415-8) and Kaeppler et al, Theor Appl Genet (1992) 84:560-6 (whisker-mediated transformation); D'Halluin et al, Plant Cell (1992) 4: 1495-505 (electroporation); Li et al., Plant Cell Rep (1993) 12:250-5; Christou and Ford Annals Botany (1995) 75:407-13 (rice) and Osjoda et al, Nat Biotechnol (1996) 14:745-50 (maize via Agrobacterium tumefaciens).

Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature. See, for example Horsch et al (1984) Science 233:496-498, and Fraley et al (1983) Proc. Natl. Acad. Sci. USA 80:4803. The virulence functions of the Agrobacterium tumefaciens host will direct the insertion of the construct and adjacent marker into the plant cell DNA when the cell is infected by the bacteria using binary T DNA vector (Bevan (1984) Nuc. Acid Res. 12:8711-8721) or the co-cultivation procedure (Horsch et al (1985) Science 227: 1229-1231). The Agrobacterium transformation system may also be used to transform, as well as transfer, DNA to monocotyledonous plants and plant cells. See Hernalsteen et al (1984) EMBO J 3:3039-3041; Hooykass-Van Slogteren et al (1984) Nature 311:763-764; Grimsley et al (1987) Nature 325: 1677-179; Boulton et al (1989) Plant Mol. Biol. 12:31-40; and Gould et al (1991) Plant Physiol. 95:426-434.

Alternatively, polynucleotides may be introduced into plants by contacting plants with a virus or viral nucleic acids. Generally, such methods involve incorporating a polynucleotide within a viral DNA or RNA molecule. In some embodiments, a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191; 5,889,190; 5,866,785; 5,589,367 and 5,316,931.

In other embodiments, an RNA polynucleotide encoding the Cas12d protein is introduced into the plant cell, which is then translated and processed by the host cell generating the protein in sufficient quantity to modify the cell (in the presence of at least one guide RNA) but which does not persist after a contemplated period of time has passed or after one or more cell divisions. Methods for introducing mRNA to plant protoplasts for transient expression are known by the skilled artisan (see for instance in Gallie, Plant Cell Reports (1993), 13; 119-122). Transient transformation methods include, but are not limited to, the introduction of polypeptides, such as a double-strand break inducing agent, directly into the organism, the introduction of polynucleotides such as DNA and/or RNA polynucleotides, and the introduction of the RNA transcript, such as an mRNA encoding a double-strand break inducing agent, into the organism. Such methods include, for example, microinjection or particle bombardment. See, for example Crossway et al, Mol. Gen. Genet. (1986) 202: 179-85; Nomura et al, Plant Sci. (1986) 44:53-8; Hepler et al, Proc. Natl. Acad. Sci. USA (1994) 91: 2176-80; and Hush et al, J. Cell Sci. (1994) 107: 775-84.

For particle bombardment or with protoplast transformation, the expression system can comprise one or more isolated linear fragments or may be part of a larger construct that might contain bacterial replication elements, bacterial selectable markers or other detectable elements. The expression cassette(s) comprising the polynucleotides encoding the guide and/or Cas12d may be physically linked to a marker cassette or may be mixed with a second nucleic acid molecule encoding a marker cassette. The marker cassette is comprised of necessary elements to express a detectable or selectable marker that allows for efficient selection of transformed cells.

In certain embodiments, it is of interest to deliver one or more components of the Cas12d CRISPR system directly to the plant cell, for example to generate non-transgenic plants. One or more of the Cas12d components may be prepared outside the plant or plant cell and delivered to the cell. For instance, the Cas12d protein can be prepared in vitro prior to introduction to the plant cell. Cas12d protein can be prepared by various methods known by one of skill in the art and include recombinant production. After expression, the Cas12d protein is isolated, refolded if needed, purified and optionally treated to remove any purification tags, such as a His-tag. Once crude, partially purified, or more completely purified Cas12d protein is obtained, the protein may be introduced to the plant cell. In particular embodiments, the Cas12d protein is mixed with guide RNA targeting the gene of interest to form a pre-assembled ribonucleoprotein, which can be delivered to a plant cell by any one or more of electroporation, bombardment, chemical transfection and other means of delivery described herein.

### Genetic Constructs of the Disclosure

The present disclosure further provides expression constructs, such as for example and not limitation an expression cassette, for expressing in a host (e.g., a plant, plant cell, or plant part) a CRISPR Cas12d system that is capable of binding to and creating a double strand break in a target site. In one embodiment, the expression constructs of the disclosure comprise a promoter operably linked to a nucleotide sequence encoding a CRISPR Cas12d gene and a promoter operably linked to a guide nucleic acid of the present disclosure. The promoter is capable of driving expression of an operably linked nucleotide sequence in a host (e.g., a plant) cell. In another embodiment, the CRISPR Cas12d gene comprises one or more transcriptional and/or translational fusions as described herein. In some embodiments, the expression cassette allows transient expression of the CRISPR/Cas12d system, while in other embodiments, the expression cassette allows the CRISPR/Cas12d system to be stably maintained within the host cell, such as for example and not limitation, by integration into the host cell genome.

A promoter is a region of DNA involved in recognition and binding of RNA polymerase and other proteins to initiate transcription. Promoters are well known in the art to be highly specific and adapted for use in particular kingdoms, genera, species, and even particular tissues within the same organism. Promoters can be constitutively active or inducible; examples of each are well known in the art. For example, a plant promoter is a promoter capable of initiating transcription in a plant cell, for a review of plant promoters, see, Potenza et al, In Vitro Cell Dev Biol (2004) 40: 1-22. A constitutive plant promoter is a promoter that is able to express the open reading frame (ORF) that it controls in all or nearly all of the plant tissues during all or nearly all developmental stages of the plant (referred to as “constitutive expression”). Constitutive promoters include, for example, the core promoter of the Rsyn7 promoter and other constitutive promoters disclosed in WO99/43838 and U.S. Pat. No. 6,072,050; the core CaMV 35S promoter (Odell et al, Nature (1985) 313:810-2); rice actin (McElroy et al, Plant Cell (1990) 2: 163-71); ubiquitin (Christensen et al, Plant Mol Biol (1989) 12:619-32; Christensen et al, Plant Mol Biol (1992) 18:675-89); pEMU (Last et al, Theor Appl Genet (1991) 81:581-8); MAS (Velten et al, EMBO J. (1984) 3:2723-30); ALS promoter (U.S. Pat. No. 5,659,026), and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611.

In some embodiments, an inducible promoter may be used. Pathogen-inducible promoters induced following infection by a pathogen include, but are not limited to those regulating expression of PR proteins, SAR proteins, beta-1, 3-glucanase, chitinase, etc. Alternatively, the sequence encoding the Cas12d endonuclease can be operably linked to a promoter that is constitutive, cell specific, or activated by alternative splicing of a suicide exon.

Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize ln2-2 promoter, activated by benzene sulfonamide herbicide safeners (De Veylder et al., Plant Cell Physiol (1997) 38:568-77), the maize GST promoter (GST-11-27, WO93/01294), activated by hydrophobic electrophilic compounds used as pre-emergent herbicides, and the tobacco PR-1 a promoter (Ono et al., Biosci Biotechnol Biochem (2004) 68:803-7) activated by salicylic acid. Other chemical-regulated promoters include steroid-responsive promoters (see, for example, the glucocorticoid-inducible promoter (Schena et al, Proc. Natl. Acad. Sci. USA (1991) 88: 10421-5; McNellis et al, Plant J (1998) 14:247-257); tetracycline-inducible and tetracycline-repressible promoters (Gatz et al., Mol Gen Genet (1991) 227:229-37; U.S. Pat. Nos. 5,814,618 and 5,789,156).

Inducible promoters can be used that allow for spatiotemporal control of gene editing or gene expression may use a form of energy. The form of energy may include but is not limited to sound energy, electromagnetic radiation, chemical energy and/or thermal energy. Examples of light inducible systems (Phytochrome, LOV domains, or cryptochrome), such as a Light Inducible Transcriptional Effector (LITE) that direct changes in transcriptional activity in a sequence-specific manner. The components of a light inducible system may include a Cpf1 CRISPR enzyme, a light-responsive cytochrome heterodimer (e.g. from Arabidopsis thaliana), and a transcriptional activation/repression domain.

Tissue-preferred promoters can be utilized to target enhanced expression within a particular plant tissue. Tissue-preferred promoters include, for example, Kawamata et al., Plant Cell Physiol (1997) 38:792-803; Hansen et al, Mol Gen Genet (1997) 254:337-43; Russell et al, Transgenic Res (1997) 6: 157-68; Rinehart et al, Plant Physiol 1 (1996) 12: 1331-41; Van Camp et al, Plant Physiol (1996) 1 12:525-35; Canevascini et al, Plant Physiol (1996) 112:513-524; Lam, Results Probl Cell Differ (1994) 20: 181-96; and Guevara-Garcia et al, Plant J (1993) 4:495-505. Leaf-preferred promoters include, for example, Yamamoto et al., Plant J (1997) 12:255-65; Kwon et al., Plant Physiol (1994) 105:357-67; Yamamoto et al, Plant Cell Physiol (1994) 35:773-8; Gotor et al, Plant J (1993) 3:509-18; Orozco et al, Plant Mol Biol (1993) 23: 1 129-38; Matsuoka et al, Proc. Natl. Acad. Sci. USA (1993) 90:9586-90; Simpson et al, EMBO J (1958) 4:2723-9; Timko et al., Nature (1988) 318:57-8. Root-preferred promoters include, for example, Hire et al., Plant Mol Biol (1992) 20:207-18 (soybean root-specific glutamine synthase gene); Miao et al., Plant Cell (1991) 3: 11-22 (cytosolic glutamine synthase (GS)); Keller and Baumgartner, Plant Cell (1991) 3: 1051-61 (root-specific control element in the GRP 1 0.8 gene of French bean); Sanger et al., Plant Mol Biol (1990) 14:433-43 (root-specific promoter of A. tumefaciens mannopine synthase (MAS)); Bogusz et al., Plant Cell (1990) 2:633-41 (root-specific promoters isolated from Parasponia andersonii and Trema tomentosa); Leach and Aoyagi, Plant Sci (1991) 79:69-76 A. rhizogenes rolC and rolD root-inducing genes); Teeri et al., EMBO J (1989) 8:343-50 (Agrobacterium wound-induced TR1′ and TR2′ genes); VfENOD-GRP3 gene promoter (Kuster et al, Plant Mol Biol (1995) 29:759-72); and rolB promoter (Capana et al, Plant Mol Biol (1994) 25:681-91; phaseolin gene (Murai et al., Science (1983) 23:476-82; Sengopta-Gopalen et al., Proc. Natl. Acad. Sci. USA (1988) 82:3320-4). See also, U.S. Pat. Nos. 5,837,876; 5,750,386; 5,633,363; 5,459,252; 5,401,836; 5,110,732 and 5,023,179.

In some embodiments, a DNA-dependent RNA polymerase II promoter or a DNA-dependent RNA polymerase III promoter is used. In some embodiments, a monocot promoter is used to drive expression in monocots. In various additional embodiments, a dicot promoter is used to drive expression in dicots.

Seed-preferred promoters include both seed-specific promoters active during seed development, as well as seed-germinating promoters active during seed germination. See, Thompson et al., BioEssays (1989) 10: 108. Seed-preferred promoters include, but are not limited to, Cim1 (cytokinin-induced message); cZ19Bl (maize 19 kDa zein); and milps (myo-inositol-1-phosphate synthase); (WO00/11177; and U.S. Pat. No. 6,225,529). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1 shrunken 2, globulin 1 oleosin, and nud. See also, WO00/12733, where seed-preferred promoters from END1 and END2 genes are disclosed.

A phenotypic marker is a screenable or selectable marker that includes visual markers and selectable markers whether it is a positive or negative selectable marker. Any phenotypic marker can be used. Specifically, a selectable or screenable marker comprises a DNA segment that allows one to identify or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), yellow-green (mNeonGreen), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as glufosinate ammonium, bromoxynil, imidazolinones, and 2,4-dichlorophenoxyacetate (2,4-D). See for example, Yarranton, Curr Opin Biotech (1992) 3:506-1 1; Christopherson et al, Proc. Natl. Acad. Sci. USA (1992) 89:6314-8; Yao et al, Cell (1992) 71:63-72; Reznikoff, Mol Microbiol (1992) 6:2419-22; Hu et al, Cell (1987) 48:555-66; Brown et al, Cell (1987) 49:603-12; Figge et al, Cell (1988) 52:713-22; Deuschle et al, Proc. Natl. Acad. Sci. USA (1989) 86:5400-4; Fuerst et al, Proc. Natl. Acad. Sci. USA (1989) 86:2549-53; Deuschle et al, Science (1990) 248:480-3; Gossen, (1993) Ph.D. Thesis, University of Heidelberg; Reines et al, Proc. Natl. Acad. Sci. USA (1993) 90: 1917-21; Labow et al, Mol Cell Biol (1990) 10:3343-56; Zambretti et al, Proc. Natl. Acad. Sci. USA (1992) 89:3952-6; Bairn et al, Proc. Natl. Acad. Sci. USA (1991) 88:5072-6; Wyborski et al, Nucleic Acids Res (1991) 19:4647-53; Hillen and Wissman, Topics Mol Struc Biol (1989) 10: 143-62; Degenkolb et al, Antimicrob Agents Chemother (1991) 35: 1591-5; Kleinschnidt et al, Biochemistry (1988) 27: 1094-104; Bonin, (1993) Ph.D. Thesis, University of Heidelberg; Gossen et al, Proc. Natl. Acad. Sci. USA (1992) 89:5547-51; Oliva et al, Antimicrob Agents Chemother (1992) 36:913-9; Hlavka et al, Handbook of Experimental Pharmacology, (1985) Vol. 78 (Springer-Verlag, Berlin); Gill et al, Nature (1988) 334:721-4.

Various selection procedures for the cells based on the selectable marker can be used, depending on the nature of the marker gene. In particular embodiments, use is made of a selectable marker, i.e., a marker which allows a direct selection of the cells based on the expression of the marker. A selectable marker can confer positive or negative selection and is conditional or non-conditional on the presence of external substrates (Miki et al. 2004, 107(3): 193-232). Most commonly, antibiotic or herbicide resistance genes are used as a marker, whereby selection is be performed by growing the engineered plant material on media containing an inhibitory amount of the antibiotic or herbicide to which the marker gene confers resistance. Examples of such genes are genes that confer resistance to antibiotics, such as hygromycin (hpt) and kanamycin (nptII), and genes that confer resistance to herbicides, such as phosphinothricin (bar), chlorsulfuron (als), aroA, glyphosate acetyl transferase (GAT) genes, phosphinothricin acetyl transferase (PAT) genes from Streptomyces species, and ACCase inhibitor-encoding genes. Detoxifying genes can also be used as a marker, with examples including an enzyme encoding a phosphinothricin acetyltransferase, phosphinothricin acetyltransferases, and hydroxyphenylpyruyate dioxygenase (HPPD) inhibitors.

Transformed plants and plant cells may also be identified by screening for the activities of a visible marker, typically an enzyme capable of processing a colored substrate (e.g., the β-glucuronidase, luciferase, B or CI genes). Such selection and screening methodologies are well known to those skilled in the art.

### Transgenic Plants, Plant Parts, Cells and Seeds of the Disclosure

In a preferred embodiment of the disclosure, transgenic plants including transgenic parts of the transgenic plant, in particular transgenic seeds and transgenic cells are provided. The transgenic parts of the transgenic plant can further include those parts which can be harvested, such as for example and not limitation, the beets for sugar beet, rice grains for rice, and corn cobs for maize.

For production of transgenic seeds carrying the integrated nucleic acid construct, the transgenic plant may be selfed. Alternatively, the transgenic plant can be crossed with a similar transgenic plant or with a transgenic plant which carries one or more nucleic acids that are different from the invented genetic constructs, or with a non-transgenic plant of known plant breeding methods to produce transgenic seeds. These seeds can be used to provide progeny generations of transgenic plants of the disclosure, comprising the integrated nucleic acid from the invented genetic constructs.

Suitable methods of transforming plant cells are known in plant biotechnology and are described herein. Transformed plant cells can be cultured to regenerate a whole plant which possesses the transformed genotype and thus the desired phenotype. Each of these methods can be used to preferentially introduce a selected nucleic acid into a vector into a plant cell to obtain a transgenic plant of the present disclosure. Transformation methods may include direct and indirect methods of transformation and are applicable for dicotyledonous and mostly for monocots. The plant can be monocotyledonous (e.g., wheat, maize, or Setaria), or the plant can be dicotyledonous (e.g., tomato, soybean, tobacco, potato, or Arabidopsis).

The methods described herein also can be utilized with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., Pinales, Ginkgoales, Cycadales and Gnetales.

The methods described herein can be utilized with dicotyledonous plants belonging, for example, to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

The methods described herein can be utilized over a broad range of plants including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea.

Transformed plant cells, including protoplasts and plastids, are selected for one or more markers which have been transformed with the nucleic acid of the disclosure into the plant and include genes that mediate preferably antibiotic resistance, such as the neomycin phosphotransferase II-mediated gene NPTII, which encodes kanamycin resistance. Alternatively, herbicide resistance genes can be used. Subsequently, the transformed cells are regenerated into whole plants. Following DNA transfer and regeneration, the plants can be checked for example the quantitative PCR for the presence of the nucleic acid of the disclosure.

In some embodiments, antibiotic resistance and/or herbicidal resistance selection markers could be co-introduced with CRISPR/Cas12d system into plant cells for targeted gene repair/correction and knock-in (gene insertion and replacement) via homologous recombination. In combination with different donor DNA fragments, the CRISPR/Cas12d system could be used to modify various agronomic traits for genetic improvement.

The cells having the introduced sequence may be grown or regenerated into plants using conventional conditions, see for example, McCormick et al, Plant Cell Rep (1986) 5:81-4. These plants may then be grown, and either pollinated with the same transformed strain or with a different transformed or untransformed strain, and the resulting progeny having the desired characteristic and/or comprising the introduced polynucleotide or polypeptide identified. Two or more generations may be grown to ensure that the polynucleotide is stably maintained and inherited, and seeds harvested.

Any plant can be used, including monocot and dicot plants. Examples of monocot plants that can be used include, but are not limited to, corn (Zea mays), rice (Oryza sativa), rye (Secale cereale), sorghum {Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum aestivum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses. Examples of dicot plants that can be used include, but are not limited to, soybean (Glycine max), canola (Brassica napus and B. campestris), alfalfa (Medicago sativa), tobacco (Nicotiana tabacum), Arabidopsis (Arabidopsis thaliana), sunflower (Helianthus annuus), sugar beet (Beta vulgaris), cotton (Gossypium arboreum), and peanut (Arachis hypogaea), tomato (Solanum lycopersicum), potato (Solanum tuberosum), etc. Additional monocots that can be used include oil palm (Elaeis guineensis), sudangrass (Sorghum x drummondii), and rye (Secale cereale). Additional dicots that can be used include safflower (Carthamus tinctorius), coffee (Coffea arabica and Coffea canephora), amaranth (Amaranthus spp.), and rapeseed (Brassica napus and Brassica napobrassica; high erucic acid and canola).

Additional non-limiting exemplary plants for use with the invented methods and compositions include Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, or any variety or subspecies belonging to one of the aforementioned plants.

Treatment Methods for Use with the Disclosure

The invented method provides a method for treating diseases and/or conditions (such as for example and not limitation, diseases caused by insects). The invented method further provides a method for preventing insect infection and/or infestation in a plant (e.g., insect resistance).

Non-limiting examples of the diseases and/or conditions treatable by the invented methods include Anthracnose Stalk Rot, Aspergillus Ear Rot, Common Corn Ear Rots, Corn Ear Rots (Uncommon), Common Rust of Corn, Diplodia Ear Rot, Diplodia Leaf Streak, Diplodia Stalk Rot, Downy Mildew, Eyespot, Fusarium Ear Rot, Fusarium Stalk Rot, Gibberella Ear Rot, Gibberella Stalk Rot, Goss's Wilt and Leaf Blight, Gray Leaf Spot, Head Smut, Northern Corn Leaf Blight, Physoderma Brown Spot, Pythium, Southern Leaf Blight, Southern Rust, and Stewart's Bacterial Wilt and Blight, and combinations thereof.

Non-limiting examples of the insects causing, directly or indirectly, diseases and/or conditions treatable by the invented methods include Armyworm, Asiatic Garden Beetle, Black Cutworm, Brown Marmorated Stink Bug, Brown Stink Bug, Common Stalk Borer, Corn Billbugs, Corn Earworm, Corn Leaf Aphid, Corn Rootworm, Corn Rootworm Silk Feeding, European Corn Borer, Fall Armyworm, Grape Colaspis, Hop Vine Borer, Japanese Beetle, Scouting for Fall Armyworm, Seedcorn Beetle, Seedcorn Maggot, Southern Corn Leaf Beetle, Southwestern Corn Borer, Spider Mite, Sugarcane Beetle, Western Bean Cutworm, White Grub, and Wireworms, and combinations thereof. The invented methods are also suitable for preventing infections and/or infestations of a plant by any such insect(s).

Further non-limiting examples of plant diseases are listed in WO 2013/046247 and incorporated herein by reference.

### Methods for Creating Nutritionally Improved Crops and Functional Foods

The Cas12d systems and methods described herein may be used to produce nutritionally improved agricultural crops. In some embodiments, the methods provided herein are adapted to generate “functional foods”, i.e. a modified food or food ingredient that may provide a health benefit beyond the traditional nutrients it contains and or “nutraceutical”, i.e. substances that may be considered a food or part of a food and provides health benefits, including the prevention and treatment of disease. The nutraceutical may be useful in the prevention and/or treatment of one or more of cancer, diabetes, cardiovascular disease, and hypertension.

For instance, a nutritionally improved agricultural crop may have induced or increased synthesis of one or more of the following compounds: carotenoids, such as a-carotene or β-carotene present in various fruits and vegetables; lutein; lycopene present in tomato and tomato products; zeaxanthin, present in citrus and maize; dietary fiber, β-glucan, fatty acids (such as omega-3, conjugated linoleic acid, GLA, and CVD); flavonoids (e.g., hydroxycinnamates present in wheat); flavonols; catechins; tannins; glucosinolates; indoles; isothiocyanates, such as sulforaphane; phenolics, such as stilbenes present in grape, caffeic acid, ferulic acid and epicatechin; plant stanols/sterols present in maize, soy, wheat and wooden oils; fructans; inulins; fructo-oligosaccharides present in Jerusalem artichoke; saponins present in soybean; phytoestrogens; lignans present in flax, rye and vegetables; diallyl sulphide; allyl methyl trisulfide; dithiolthiones; and tannins, such as proanthocyanidins.

Induction or increased synthesis can occur by directing introducing one or more genes encoding proteins involved in the synthesis of the above compounds. Alternatively, the metabolism of the plant can be modified so as to increase production of one or more of the above compounds. For example, a plant can be engineered to express an antisense gene of stearyl-ACP desaturase to increase stearic acid content of the plant. A plant can be engineered to express mutated forms of DNA to block degradation of one of the above compounds. Arabidopsis thaliana can be engineered to express Tfs CI and R under the control of a strong promoter to bring about a high accumulation rate of anthocyanins. See, Bruce et al., 2000, Plant Cell 12:65-80. Increasing expression of Tf RAP2.2 and its interacting partner SINAT2 can increase carotenogenesis in Arabidopsis leaves. Expressing the Tf Dofl in Arabidopsis can induce the up-regulation of genes encoding enzymes for carbon skeleton production, a marked increase of amino acid content, and a reduction of the Glc level.

The methods provided herein may be used to generate plants with a reduced level of allergens. In particular embodiments, the methods comprise modifying expression of one or more genes responsible for the production of plant allergens. In some embodiments, Cas12d can be used to disrupt or down regulate expression of a Lol p5 gene in a plant cell, such as a ryegrass plant cell and regenerating a plant therefrom so as to reduce allergenicity of the pollen of said plant. The Cas12d system and methods described herein can be used to identify and then edit or silence genes encoding allergenic proteins of such legumes. Some such genes may have been identified in peanuts, soybeans, lentils, peas, lupin, green beans, and mung beans. See, Nicolaou et al., Current Opinion in Allergy and Clinical Immunology 201 1; 11(3):222).

### Methods for Enhancing Biofuel Production

The Cas12d systems and methods described herein may be used to enhance biofuel production in plants. Renewable biofuels can be extracted from organic matter whose energy has been obtained through a process of carbon fixation or are made through the use or conversion of biomass. Such biomass can be used directly for biofuels or can be converted to convenient energy containing substances by thermal conversion, chemical conversion, and biochemical conversion. At least two types of biofuels can be produced: bioethanol and biodiesel. Bioethanol is mainly produced by the sugar fermentation process of cellulose (starch), which is mostly derived from maize and sugar cane. Biodiesel on the other hand is mainly produced from oil crops such as rapeseed, palm, and soybean.

The methods using the Cas12d CRISPR system as described herein may be used to alter the properties of the cell wall in order to facilitate access by key hydrolyzing agents for a more efficient release of sugars for fermentation. In particular embodiments, the biosynthesis of cellulose and/or lignin are modified. Cellulose is the major component of the cell wall. The biosynthesis of cellulose and lignin are co-regulated. By reducing the proportion of lignin in a plant the proportion of cellulose can be increased. In particular embodiments, the methods described herein are used to downregulate lignin biosynthesis in the plant so as to increase fermentable carbohydrates. More particularly, the methods described herein are used to downregulate at least a first lignin biosynthesis gene selected from the group consisting of 4-coumarate 3-hydroxylase (C3H), phenylalanine ammonia-lyase (PAL), cinnamate 4-hydroxylase (C4H), hydroxycinnamoyl transferase (HCT), caffeic acid O-methyltransferase (COMT), caffeoyl CoA 3-O-methyltransferase (CCoAOMT), ferulate 5-hydroxylase (F5H), cinnamyl alcohol dehydrogenase (CAD), cinnamoyl CoA-reductase (CCR), 4-coumarate-CoA ligase (4CL), monolignol-lignin-specific glycosyltransferase, and aldehyde dehydrogenase (ALDH) as disclosed in WO2008/064289. The methods disclosed herein can be used to generate mutations in homologs to CaslL to reduce polysaccharide acetylation.

Additional methods and compositions for use with the present disclosure are found in US2015/0152398, US2016/0145631, US2015/089681, US20200255858, WO2016/205749, and WO2016/196655, which are each incorporated herein by reference in their entireties.

### EMBODIMENTS

The following numbered embodiments form part of the disclosure.

1. A method for modifying expression of at least one chromosomal or extrachromosomal gene in a eukaryotic cell, said method comprising introducing into the cell: (a) (i) a Clustered Regularly Interspersed Short Palindromic Repeats (CRISPR) RNA (crRNA) and a short-complementarity untranslated RNA (scoutRNA) or DNA encoding the crRNA and scoutRNA, or (ii) a chimeric cr/scoutRNA hybrid (sgRNA) or DNA encoding the sgRNA, wherein the crRNA or the sgRNA comprises a sequence at least partially complementary to a target sequence within the gene or which can hybridize to a target sequence within the gene; and (b) a CRISPR Cas12d endonuclease molecule, wherein said CRISPR/Cas12d endonuclease is capable of binding to the sequence to which the crRNA or sgRNA is targeted; wherein the eukaryotic cell is optionally a mammalian, yeast, fish, or plant cell.

2. The method of embodiment 1 wherein the crRNA comprises a repeat sequence of about 11 nucleotides and a spacer sequence of about 18 nucleotides, wherein the spacer sequence interacts with the target nucleic acid.

3. The method of embodiment 1 or embodiment 2, wherein: (i) the crRNA or scoutRNA or sgRNA comprises unconventional and/or modified nucleotides and/or comprises unconventional and/or modified backbone chemistries; (ii) wherein the scoutRNA or sgRNA comprises the RNA molecule of SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56; and/or (iii) wherein the sgRNA comprises the RNA molecule of SEQ ID NO: 5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59.

4. The method of embodiment 3, wherein crRNA or scoutRNA or sgRNA comprises one or more modifications selected from the group consisting of locked nucleic acid (LNA) bases, internucleotide phosphorothioate bonds in the backbone, 2′-O-Methyl RNA bases, unlocked nucleic acid (UNA) bases, 5-Methyl dC bases, 5-hydroxybutynl-2′-deoxyuridine bases, 5-nitro indole bases, deoxyinosine bases, 8-aza-7-deazaguanosine bases, dideoxy-T at the 5′ end, inverted dT at the 3′ end, and dideoxycytidine at the 3′ end.

5. The method of embodiment 1 or embodiment 2, wherein the crRNA, scoutRNA or sgRNA is introduced into the cell as a DNA molecule encoding said RNA and is operably linked to a promoter directing production of said RNA in the cell.

6. The method of any one of embodiments 1-5, wherein the CRISPR/Cas12d endonuclease molecule comprises the amino acid sequence of SEQ ID NO: 1 or a sequence having at least 85% sequence identity to SEQ ID NO: 1.

7. The method of any one of embodiment 6, wherein the CRISPR/Cas12d endonuclease molecule is capable of introducing a double stranded break or a single stranded break at, within, or near the sequence to which the crRNA or sgRNA is targeted.

8. The method of any one of embodiments 1-5, wherein the CRISPR/Cas12d endonuclease molecule is a dCas12d.

9. The method of embodiment 8, wherein the dCas12d comprises a mutation of one or more of residues selected from the group consisting of D775, E971, D1198, C1053, C1056, C1186, and C1191 of SEQ ID NO: 1, optionally wherein the dCas12 molecule comprises one or more mutations selected from the group consisting of D775A, E971A, D1198A, C1053A, C1056A, C1186A, and C1191A of SEQ ID NO: 1.

10. The method of any one of embodiments 1-9, wherein the CRISPR/Cas12d endonuclease molecule is modified so as to be active at a different temperature than its optimal temperature prior to modification.

11. The method of embodiment 10, wherein the modified CRISPR/Cas12d endonuclease molecule is active at temperatures suitable for growth and culture of eukaryotes or eukaryotic cells, wherein the eukaryotes are optionally mammals, yeasts, or plants and wherein the eukaryotic cell is optionally a mammalian, yeast, fungal, fish, or plant cell.

12. The method of embodiment 10, wherein the modified CRISPR/Cas12d endonuclease molecule is active at a temperature from about 20° C. to about 35° C.

13. The method of embodiment 12, wherein the modified CRISPR/Cas12d endonuclease molecule is active at a temperature from about 23° C. to about 32° C.

14. The method of embodiment 13, wherein the modified CRISPR/Cas12d endonuclease molecule is active at a temperature from about 25° C. to about 28° C.

15. The method of any one of embodiments 1-14, wherein: (i) the CRISPR/Cas12d endonuclease molecule is delivered to the cell as a DNA molecule comprising a CRISPR/Cas12d endonuclease coding sequence operably linked to a promoter directing production of said CRISPR/Cas12d endonuclease in the cell; (ii) the crRNA and scoutRNA is delivered to the cell as a DNA molecule comprising one or more sequences encoding the crRNA and scoutRNA, wherein the one or more sequences encoding the crRNA and scoutRNA are operably linked to one or more promoter(s) directing production of the crRNA and scoutRNA in the cell; or (iii) both (i) and (ii).

16. The method of embodiment 15, wherein the DNA molecule is transiently present in the cell.

17. The method of embodiment 15, wherein the DNA molecule is stably incorporated into the nuclear or plastidic genomic sequence of the cell or a progenitor cell, thereby providing heritable expression of the CRISPR/Cas12d endonuclease molecule.

18. The method of any one of embodiments 1-14, wherein the CRISPR/Cas12d endonuclease molecule is delivered to the cell as an mRNA molecule encoding said CRISPR/Cas12d endonuclease.

19. The method of any one of embodiments 1-14, wherein the CRISPR/Cas12d endonuclease molecule is delivered to the cell as a protein.

20. The method of any one of embodiments 1-19, wherein the CRISPR/Cas12d endonuclease molecule comprises one or more elements selected from the group consisting of localization signals, detection tags, detection reporters, and purification tags.

21. The method of embodiment 20, wherein the CRISPR/Cas12d endonuclease molecule comprises one or more localization signals.

22. The method of any one of embodiments 1-21 wherein the CRISPR/Cas12d endonuclease molecule comprises at least one additional protein domain with enzymatic activity.

23. The method of embodiment 22, wherein the at least one additional protein domain has an enzymatic activity selected from the group consisting of exonuclease, helicase, repair of DNA double-stranded breaks, transcriptional (co-)activator, transcriptional (co-)repressor, methylase, demethylase, and any combinations thereof.

24. The method of any one of embodiments 1-4, 6-14, and 19-23, wherein the method comprises delivering a preassembled complex comprising the CRISPR/Cas12d endonuclease molecule loaded with the crRNA/scoutRNA or sgRNA prior to introduction into the cell.

25. The method of any one of embodiments 5 and 15-17, wherein the promoter is selected from the group consisting of constitutive promoters, inducible promoters, and cell-type or tissue-type specific promoters.

26. The method of any one of embodiments 5 and 15-17, wherein the promoter is activated by alternative splicing of a suicide exon.

27. The method of any one of embodiments 1-26, wherein the DNA or RNA is delivered to the cell by a method selected from the group consisting of microparticle bombardment, polyethylene glycol (PEG) mediated transformation, electroporation, pollen-tube mediated introduction into zygotes, and delivery mediated by one or more cell-penetrating peptides (CPPs).

28. The method of any one of embodiments 1-26, wherein the DNA is delivered to the cell by bacteria-mediated transformation.

29. The method of embodiment 28, wherein the DNA is delivered to the cell in a T-DNA, and wherein the delivery is via Agrobacterium or Ensifer.

30. The method of any one of embodiments 1-26, wherein the DNA or RNA is delivered to the cell by a virus.

31. The method of embodiment 30, wherein the virus is a geminivirus or a tobravirus.

32. The method of any one of embodiments 1-31 wherein the plant is monocotyledonous.

33. The method of any one of embodiments 1-31 wherein the plant is dicotyledonous.

34. The method of any one of embodiments 1-31, wherein: (i) the eukaryotic cell is a mammalian cell optionally selected from the group consisting of a human, non-human primate, bovine, porcine, murine, canine, feline, equine, rodent, and an ungulate cell; (ii) the eukaryotic cell is a yeast cell optionally selected from the group consisting of a Saccharomyces sp., Candida, Endomycopsis, Brettanomyces sp., Candida sp., Cryptococcus sp., Debaromyces sp, Hanseniaspora sp., Hansenula sp., Kluyveromyces sp., Pichia sp., Rhodotorula sp., Torulaspora sp., Schizosaccharomyces sp., and Zygosaccharomyces sp. cell; (iii) the eukaryotic cell is a fungal cell optionally selected from the group consisting of a Aspergillus sp., Fusarium sp., Penicillium sp., Paecilomyces sp., Mucor sp., Rhizopus sp., and a Trichoderma sp. cell; (iv) the eukaryotic cell is a fish cell optionally selected from the group consisting of a salmonid, cichlid, silurid, and cyprinid cell, or (v) the eukaryotic cell is a plant cell optionally derived from a species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, and any variety or subspecies belonging to one of the aforementioned plants.

35. The method of any one of embodiments 1-34, wherein the target sequence is selected from the group consisting of an acetolactate synthase (ALS) gene, an enolpyruvylshikimate phosphate synthase gene (EPSPS) gene, male fertility genes, male sterility genes, female fertility genes, female sterility genes, male restorer genes, female restorer genes, genes associated with the traits of sterility, genes associated with the traits of fertility, genes associated with herbicide resistance, genes associated with herbicide tolerance, genes associated with fungal resistance, genes associated with viral resistance, genes associated with insect resistance, genes associated with drought tolerance, genes associated with chilling tolerance, genes associated with cold tolerance, genes associated with nitrogen use efficiency, genes associated with phosphorus use efficiency, genes associated with water use efficiency and genes associated with crop or biomass yield, and any mutants of such genes.

36. The method of embodiment 35, wherein male sterility gene is selected from the group consisting of MS45, MS26 and MSCA1.

37. A eukaryotic cell modified by the method of any one of embodiments 1-36, wherein the eukaryotic cell is optionally a mammalian, yeast, fungal, fish, or plant cell.

38. Cells, whole eukaryotic organisms, or progeny thereof derived from the cell of embodiment 37.

39. A composition comprising: (a) (i) a Clustered Regularly Interspersed Short Palindromic Repeats (CRISPR) RNA (crRNA) and a short-complementarity untranslated RNA (scoutRNA), or (ii) a chimeric cr/scoutRNA hybrid (sgRNA), wherein the crRNA or the sgRNA is targeted to a chromosomal or extrachromosomal plant gene sequence; and/or (b) a CRISPR/Cas12d endonuclease molecule, wherein said CRISPR/Cas12d endonuclease is capable of binding to the sequence to which the crRNA or sgRNA is targeted at temperatures suitable for growth and culture of a eukaryote or eukaryotic cell wherein the eukaryote is optionally a mammal, yeast, fungus, or plant and wherein the eukaryotic cell is optionally a mammalian, yeast, fungal, fish, or plant cell.

40. The composition of embodiment 39, wherein the crRNA comprises a repeat sequence of about 11 nucleotides and a spacer sequence of about 18 nucleotides, wherein the spacer sequence interacts with the target nucleic acid.

41. The composition of embodiment 39 or embodiment 40, wherein: (i) the crRNA or scoutRNA or sgRNA comprises unconventional and/or modified nucleotides and/or comprises unconventional and/or modified backbone chemistries; (ii) wherein the scoutRNA or sgRNA comprises the RNA molecule of SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56; and/or (iii) wherein the sgRNA comprises the RNA molecule of SEQ ID NO: 5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59.

42. The composition of embodiment 41 wherein crRNA or scoutRNA or sgRNA comprises one or more modifications selected from the group consisting of locked nucleic acid (LNA) bases, internucleotide phosphorothioate bonds in the backbone, 2′-O-Methyl RNA bases, unlocked nucleic acid (UNA) bases, 5-Methyl dC bases, 5-hydroxybutynl-2′-deoxyuridine bases, 5-nitro indole bases, deoxyinosine bases, 8-aza-7-deazaguanosine bases, dideoxy-T at the 5′ end, inverted dT at the 3′ end, and dideoxycytidine at the 3′ end.

43. The composition of any one of embodiments 39-42, wherein the CRISPR/Cas12d endonuclease molecule comprises the amino acid sequence of SEQ ID NO: 1 or a sequence having at least 85% sequence identity to SEQ ID NO: 1.

44. The composition of any one of embodiments 39-42, wherein the CRISPR/Cas12d endonuclease molecule is capable of introducing a double stranded break or a single stranded break at, within, or near the sequence to which the crRNA or sgRNA is targeted.

45. The composition of any one of embodiments 39-42, wherein the CRISPR/Cas12d endonuclease molecule is a dCas12d.

46. The composition of any one of embodiment 45, wherein the dCas12d comprises a mutation of one or more of residues D775, E971, D1198, C1053, C1056, C1186, and C1191 of SEQ ID NO: 1.

47. The composition of any one of embodiments 39-46, wherein the CRISPR/Cas12d endonuclease molecule is modified so as to be active at a different temperature than its optimal temperature prior to modification.

48. The composition of embodiment 47, wherein the modified CRISPR/Cas12d endonuclease molecule is active at temperatures suitable for growth and culture of a eukaryote or eukaryotic cell, wherein the eukaryote is optionally a mammal, yeast, or plant and wherein the eukaryotic cell is optionally a mammalian, yeast, fungal, fish, or plant cell.

49. The composition of embodiment 47, wherein the modified CRISPR/Cas12d endonuclease molecule is active at a temperature from about 20° C. to about 35° C.

50. The composition of embodiment 49, wherein the modified CRISPR/Cas12d endonuclease molecule is active at a temperature from about 23° C. to about 32° C.

51. The composition of embodiment 50, wherein the modified CRISPR/Cas12d endonuclease molecule is active at a temperature from about 25° C. to about 28° C.

52. The composition of any one of embodiments 39-51 wherein the CRISPR/Cas12d endonuclease molecule comprises one or more elements selected from the group consisting of localization signals, detection tags, detection reporters, and purification tags.

53. The composition of any one of embodiments 39-52, wherein the CRISPR/Cas12d endonuclease molecule is modified to express nickase activity or to have a nucleic acid targeting activity without any nickase or endonuclease activity.

54. The composition of any one of embodiments 39-53, wherein the CRISPR/Cas12d endonuclease molecule comprises at least one additional protein domain with enzymatic activity.

55. The composition of embodiment 54, wherein the at least one additional protein domain has an enzymatic activity selected from the group consisting of exonuclease, helicase, repair of DNA double-stranded breaks, transcriptional (co-)activator, transcriptional (co-)repressor, methylase, demethylase, and any combinations thereof.

56. The composition of any one of embodiments 39-55, wherein the target sequence is a plant sequence selected from the group consisting of an acetolactate synthase (ALS) gene, an enolpyruvylshikimate phosphate synthase gene (EPSPS) gene, male fertility genes, male sterility genes, female fertility genes, female sterility genes, male restorer genes, female restorer genes, genes associated with the traits of sterility, genes associated with the traits of fertility, genes associated with herbicide resistance, genes associated with herbicide tolerance, genes associated with fungal resistance, genes associated with viral resistance, genes associated with insect resistance, genes associated with drought tolerance, genes associated with chilling tolerance, genes associated with cold tolerance, genes associated with nitrogen use efficiency, genes associated with phosphorus use efficiency, genes associated with water use efficiency and genes associated with crop or biomass yield, and any mutants of such genes.

57. The composition of embodiment 56, wherein male sterility gene is selected from the group consisting of MS45, MS26 and MSCA1.

58. The composition of any one of embodiments 39-57, wherein the plant is monocotyledonous.

59. The composition of any one of embodiments 39-57, wherein the plant is dicotyledonous.

60. The composition of any one of embodiments 39-57, wherein: (i) the eukaryotic cell is a mammalian cell optionally selected from the group consisting of a human, non-human primate, bovine, porcine, murine, canine, feline, equine, rodent, and an ungulate cell; (ii) the eukaryotic cell is a yeast cell optionally selected from the group consisting of a Saccharomyces sp., Candida, Endomycopsis, Brettanomyces sp., Candida sp., Cryptococcus sp., Debaromyces sp, Hanseniaspora sp., Hansenula sp., Kluyveromyces sp., Pichia sp., Rhodotorula sp., Torulaspora sp., Schizosaccharomyces sp., and Zygosaccharomyces sp. cell; (iii) the eukaryotic cell is a fungal cell optionally selected from the group consisting of a Aspergillus sp., Fusarium sp., Penicillium sp., Paecilomyces sp., Mucor sp., Rhizopus sp., and a Trichoderma sp. cell; (iv) the eukaryotic cell is a fish cell optionally selected from the group consisting of a salmonid, cichlid, silurid, and cyprinid cell; or (v) the eukaryotic cell is a plant cell is derived from a species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, and any variety or subspecies belonging to one of the aforementioned plants.

61. A kit comprising: (a) (i) a Clustered Regularly Interspersed Short Palindromic Repeats (CRISPR) RNA (crRNA) and a short-complementarity untranslated RNA (scoutRNA), or (ii) a chimeric cr/scoutRNA hybrid (sgRNA), wherein the crRNA or the sgRNA is targeted to a sequence within a plant gene; (b) a CRISPR Cas12d endonuclease molecule, wherein said CRISPR Cas12d endonuclease is capable of introducing a double stranded break or a single stranded break at or near the sequence to which the crRNA or sgRNA is targeted at temperatures suitable for growth and culture of a eukaryote or eukaryotic cell, wherein the eukaryote is optionally a mammal, yeast, or plant and wherein the eukaryotic cell is optionally a mammalian, yeast, fungal, fish, or plant cell, and optionally (c) instructions for use.

62. A kit comprising: (a) (i) a nucleic acid molecule encoding Clustered Regularly Interspersed Short Palindromic Repeats (CRISPR) RNA (crRNA) and a short-complementarity untranslated RNA (scoutRNA), or (ii) a nucleic acid molecule encoding a chimeric cr/scoutRNA hybrid (sgRNA), wherein the crRNA or the sgRNA is targeted to a sequence within a plant gene; (b) a nucleic acid molecule encoding CRISPR Cas12d endonuclease molecule, wherein said CRISPR/Cas12d endonuclease is capable of introducing a double stranded break or a single stranded break at or near the sequence to which the crRNA or sgRNA is targeted at temperatures suitable for growth and culture of a eukaryote or eukaryotic cell, wherein the eukaryote is optionally a mammal, yeast, or plant and wherein the eukaryotic cell is optionally a mammalian, yeast, fungal, fish, or plant cell, and optionally (c) instructions for use.

63. A kit comprising: (a) (i) a nucleic acid molecule encoding Clustered Regularly Interspersed Short Palindromic Repeats (CRISPR) RNA (crRNA) and a nucleic acid molecule encoding a short-complementarity untranslated RNA (scoutRNA), or (ii) a nucleic acid molecule encoding a chimeric cr/scoutRNA hybrid (sgRNA), wherein the crRNA or the sgRNA is targeted to a sequence within a plant gene; (b) a nucleic acid molecule encoding CRISPR/Cas12d endonuclease molecule, wherein said CRISPR/Cas12d endonuclease is capable of introducing a double stranded break or a single stranded break at or near the sequence to which the crRNA or sgRNA is targeted at temperatures suitable for growth and culture of a eukaryote or eukaryotic cell, wherein the eukaryote is optionally a mammal, yeast, or plant and wherein the eukaryotic cell is optionally a mammalian, yeast, fungal, fish, or plant cell, and optionally (c) instructions for use.

64. The kit of any one of embodiments 61, 62, or 63, wherein: (i) the scoutRNA or sgRNA comprises the RNA molecule of SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56; or (ii) wherein the sgRNA comprises the RNA molecule of SEQ ID NO: 5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59.

65. A nucleic acid comprising an sgRNA or a DNA encoding an sgRNA for a Cas12d nuclease, wherein the sgRNA comprises (i) SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56; or (ii) a spacer sequence directed to a heterologous eukaryotic DNA target sequence and a scout RNA comprising an RNA molecule of SEQ ID NO: 5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59.

66. A dCas12d molecule comprising a mutation of one or more of residues selected from the group consisting of D775, E971, D1198, C1053, C1056, C1186, and C1191 of SEQ ID NO: 1, optionally wherein the dCas12 molecule comprises one or more mutations selected from the group consisting of D775A, E971A, D1198A, C1053A, C1056A, C1186A, and C1191A of SEQ ID NO: 1.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1: Cassettes for Plant-Optimized Expression of Cas12d and for Measuring Endonuclease Activity

To test activity of the Cas12d endonuclease in plant cells, SEQ ID NO: 1 is fused with a short flexible linker and N and C terminal NLSs (SEQ ID NO: 2). To demonstrate the activity of the 2NLS-CRISPR/Cas12d endonuclease in plant cells, this optimized protein is reverse-translated with codon usage for high expression in plants and then is placed in a strong constitutive expression cassette. A similar cassette is designed for expression of a 2NLS-CRISPR/Cas12d endonuclease with a C-terminal translational fusion to the green fluorescent reporter. These expression cassettes are cloned into a minimal plasmid vector backbone, such as a pBlueScript® backbone.

Another plasmid is generated as a vector for co-delivery of episomal targets for testing the endonuclease activity. It contains a strong constitutive expression cassette for a tdTomato fluorescent reporter, followed by a cloning site for the endonuclease target followed by a mNeonGreen coding sequence that would be out of frame relative to the tdTomato reporter. Endonuclease cleavage of the target site results in NHEJ repair, and some frequency of those repair events will generate frameshifts that cause expression of the mNeonGreen protein. Relative cleavage efficiency under different conditions, or of different nucleases, or of different guide-RNAs is measured by comparing the populations of cells expressing tdTomato and mNeonGreen relative to the populations of cells expressing tdTomato alone. This type of test construct is commonly referred to as a “traffic light reporter” (TLR).

### Example 2: Proper Subcellular Localization of Expressed 2NLS-CRISPR Cas12d and Cutting of an Episomal Target

To demonstrate robust expression and proper subcellular localization of the 2NLS-CRISPR/Cas12d plant-optimized gene, a plasmid containing the 2NLS-CRISPR/Cas12d-mNeonGreen expression cassette is transformed with PEG into protoplasts isolated from young leaves of corn and Nicotiana benthamiana plants and monitored for subcellular accumulation. A strong nuclear signal of the mNeonGreen reporter indicates robust expression and proper subcellular localization of the endonuclease protein.

To demonstrate activity of CRISPR Cas12d in monocot and dicot plant cells and at plant-optimized temperatures, protoplasts are isolated from young leaves of corn and Nicotiana benthamiana plants and transformed with vectors containing the 2NLS-CRISPR/Cas12d expression cassette and the TLR with the endonuclease target. In addition, 5′-phosphorylated, single-stranded RNA of various lengths is cotransformed to serve as guide-RNA for the appropriate target sequences. After transformation, cells are incubated for at least 24 hours at various temperatures between 18° C. and 37° C. (25° C.-28° C. being the optimal temperature for plant growth). Relative nuclease activity is assessed by flow cytometry to compare the population of cells expressing tdTomato and mNeonGreen relative to the population of cells expressing tdTomato alone.

### Example 3: Targeted Mutations of Chromosomal Sites by CRISPR/Cas12d in Protoplasts

To demonstrate the utility of CRISPR Cas12d for inducing targeted mutations at chromosomal targets, protoplasts are isolated from young leaves of corn plants and transformed with vectors containing the 2NLS-CRISPR/Cas12d or 2NLS-CRISPR/Cas12d-mNeonGreen expression cassettes. In addition, 5′-phosphorylated, single-stranded RNA is cotransformed to serve as guide-RNA for the appropriate target sequences in the corn genome. Targeted mutations are identified by PCR-based assays, by targeted Next Generation Sequencing (NGS; also known as deep sequencing) of the PCR-amplified target, or by loss of signal from an integrated tdTomato fluorescent reporter.

To demonstrate the utility of CRISPR/Cas12d for inducing multiplex editing events at chromosomal targets, the same experiment is repeated with cotransformation of two 5′-phosphorylated, single-stranded guide-RNA molecules. Targeted mutations are identified by PCR-based assays, by targeted NGS of the PCR-amplified target, or by loss of signal from an integrated tdTomato fluorescent reporter.

### Example 4: Targeted Mutagenesis of Chromosomal Sites by CRISPR/Cas12d in Regenerative Tissues Followed by Plant Regeneration and Inheritance of Mutations

To demonstrate the use of CRISPR Cas12d for generation of heritable gene editing events, a vector containing an herbicide selection marker and a vector containing the 2NLS-CRISPR/Cas12d expression cassette are bombarded into corn callus tissue, together with 5′-phosphorylated, single-stranded RNA to serve as guide-RNA against a chromosomal target. Plantlets are regenerated from the bombarded tissue and screened by phenotypic, PCR-based, and sequencing assays for mutations at the chromosomal target. Plants harboring targeted mutations are selfed and the progeny screened for inheritance of the mutations.

### Example 5: Use of CRISPR/Cas12d for Gene Editing in Protoplasts

To demonstrate the utility of CRISPR/Cas12d for gene editing at chromosomal targets in plant cells, protoplasts are isolated from young leaves of corn plants and transformed with vectors containing the 2NLS-CRISPR/Cas12d expression cassette, a 5′-phosphorylated, single-stranded RNA to serve as guide-RNA for the appropriate chromosomal target sequence, and a DNA repair template for proper repair of the chromosomal target. Gene editing is assessed by flow cytometry to identify the number of cells expressing a fluorescent reporter signal derived from targeted repair by the template. Proper repair is confirmed by PCR amplification and sequencing.

### Example 6: Use of Guide-RNA Containing Modified Bases for Targeted Mutagenesis in Protoplasts with CRISPR/Cas12d

To demonstrate the use of CRISPR/Cas12d in combination with guide-RNAs containing modified bases, protoplasts are isolated from young leaves of corn plants and transformed with vectors containing the 2NLS-CRISPR/Cas12d expression cassette and with or without the TLR with the endonuclease target. In addition, 5′-phosphorylated, single-stranded RNA containing modified bases is cotransformed to serve as guide-RNA for the appropriate target sequences.

Relative nuclease activity using guide-RNAs with and without various modifications is assessed by flow cytometry to compare the population of cells expressing tdTomato and mNeonGreen relative to the population of cells expressing tdTomato alone. Nuclease activity at chromosomal targets is assessed by PCR-based assays, by targeted NGS of the PCR-amplified target, or by loss of signal from an integrated tdTomato fluorescent reporter.

### Example 7: Use of Guide-RNA Containing Modified Bases for Targeted Mutagenesis in Maize Protoplasts with CRISPR/Cas12d

This example illustrates use of the Cas12d.15 endonuclease comprising the amino acid sequence of SEQ ID NO: 1 to edit genes in maize protoplasts.

Maize protoplasts were prepared using the following mesophyll protoplast preparation protocol (modified from one publicly available at molbio[dot]mgh[dot]harvard.edu/sheenweb/protocols_reg[dot]html). An enzyme solution containing 0.6 molar mannitol, 10 millimolar MES pH 5.7, 1.5% cellulase R10, and 0.3% macerozyme R10 is prepared and heated at 50-55 degrees Celsius for 10 minutes to inactivate proteases and accelerate bringing the enzyme into solution. The enzyme solution was cooled to room temperature before adding 1 millimolar CaCl₂, 5 millimolar β-mercaptoethanol, and 0.1% bovine serum albumin and passed through a 0.45 micrometer filter. A washing solution containing 0.6 molar mannitol, 4 millimolar MES pH 5.7, and 20 millimolar KCl is prepared.

Second leaves of the monocot plant (e. g., maize) were obtained and the middle 6-8 centimeters of leave were cut out. Ten leaf sections were stacked and cut into 0.5 millimeter-wide strips without bruising the leaves. The leaf strips were completely submerged in the enzyme solution in a petri dish, covered with aluminum foil, and exposed to a vacuum for 30 minutes to infiltrate the leaf tissue. The dish was transferred to a platform shaker and incubated for an additional 2.5 hours' digestion with gentle shaking (40 rpm). After digestion, the enzyme solution (now containing protoplasts) was carefully transferred using a serological pipette through a 35 micrometer nylon mesh into a round-bottom tube; rinsed with 5 milliliters of washing solution and filtered through the mesh as well. The protoplast suspension was centrifuged at 1200 rpm, 2 minutes in a swing-bucket centrifuge. As much of the supernatant as possible was aspirated off without touching the pellet, which was then gently washed once with 20 milliliters washing buffer followed by careful removal of the supernatant. The pellet was gently resuspended by swirling in a small volume of washing solution and then resuspended in 10-20 milliliters of washing buffer. The tube was placed upright on ice for 30 minutes-4 hours (no longer). After resting on ice, the supernatant was removed by aspiration and the pellet with resuspended 2-5 milliliters of washing buffer. The concentration of protoplasts was measured using a hemocytometer and the concentration adjusted to 2×10\hat{ }5 protoplasts/milliliter with washing buffer.

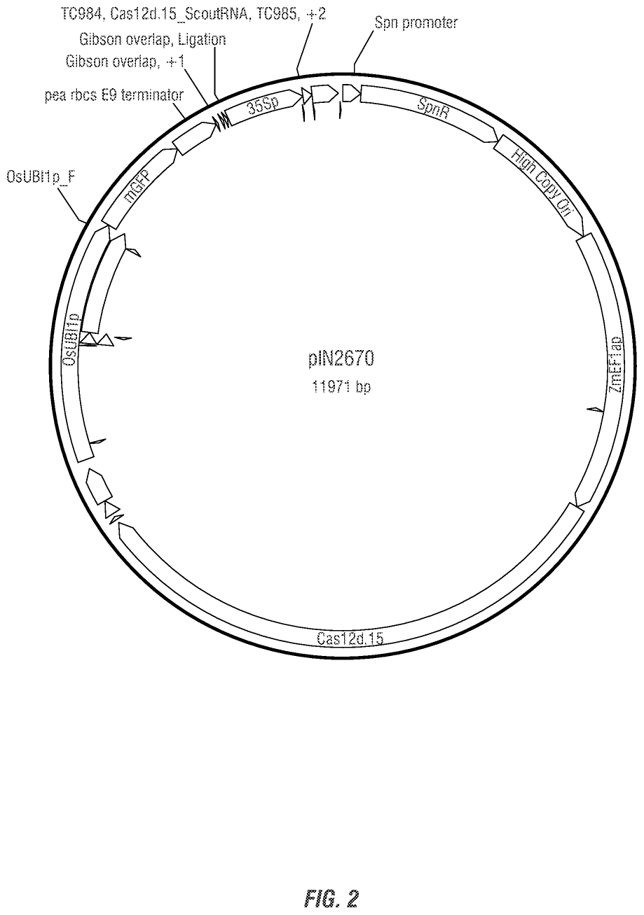

Plasmid pIN2670 (FIG. 2) was constructed to express Cas12d.15 (SEQ ID NO:1) endonuclease for testing in maize cells. The sequence is made up of Cas12d.15 fused with a nuclear localization signal (NLS) and 3×HA epitope tags at the C terminus. The vector also contains GFP and scoutRNA expressing modules, but is missing a crRNA cassette to be a fully functional editing vector. The scout RNA sequence was SEQ ID NO: 3. The crRNA sequence was GCGAUGAAGGCNNNNNNNNNNNNNNNNNN (SEQ ID NO:4), in which the respective N residues are RNA equivalents of the Cas12d spacers in Table 2 below. Unmodified and Alt-R modified crRNA and scoutRNA were obtained from Integrated DNA Technologies, Coralville, Iowa, USA.

TABLE 2
Cas12d spacer sequences, endogenous target genes, and associated
PAM sites

| Cas12d | | | Cas12d |
|---|---|---|---|
| Target | | | PAM |
| id | Cas12d spacer (18 nt) | Endogenous Genomic Target ¹ | (2 bp) |
| g5 | GAGCACGGAACGAGCAAG | Zm00007a00045852_rc | TA |
| | (SEQ ID NO: 6) | | |
| g6 | ATGATGAAGGAGTGGGCG | B104_chr1:135168000 . . . 135181999 | TG |
| | (SEQ ID NO: 7) | | |
| g9 | GAGCAAGAAGATAACGGG | Zm00007a00006349_rc | TG |
| | (SEQ ID NO: 8) | | |

¹ Endogenous genomic targets are identified by their reference numbers in the world wide web internet site “maizegdb.org/” and “maizegdb.org/gbrowse/maize_b104_chr.” Portwood et al. Nucleic Acids Res. 2018 Nov. 8. doi: 10.1093/nar/gky1046

Protoplasts were co-transfected with plasmid and/or RNAs with following delivery protocol (modified from one publicly available at molbio[dot]mgh[dot]harvard.edu/sheenweb/protocols_reg[dot]html). A polyethylene glycol (PEG) solution containing 40% PEG 4000, 0.2 molar mannitol, and 0.1 molar CaCl₂ is prepared. An incubation solution containing 154 mM NaCl, 125 mM CaCl₂, 5 mM KCl, 2 mM MES, pH 5.8, is also prepared. A mixture of Cas12d-expressing and scoutRNA-expressing plasmid, crRNA, and optionally scoutRNA by mixing the scoutRNA and crRNA (obtainable e. g., as custom-synthesized Alt-R™ CRISPR crRNA and scoutRNA oligonucleotides from Integrated DNA Technologies, Coralville, IA), and purified circular plasmid DNA. Nucleic acid solutions were prepared at a concentrations of 120 micromolar crRNA (if delivered alone), 240 micromolar crRNA and 240 micromolar scoutRNA (when delivered together). Total amount added per sample of each component was 1.2 nanomoles. Plasmid was concentrated to 2 micrograms/microliter. Each sample received 20 micrograms of plasmid DNA. The plasmid and/or RNA solutions were added to 100 microliters of monocot protoplasts (prepared as described above) in a microfuge tube with 5 micrograms salmon sperm DNA (VWR Cat. No.: 95037-160) and an equal volume of the PEG solution then gently mixed by tapping. After 5 minutes, the mixture was diluted with 880 microliters of washing buffer and mixed gently by inverting the tube. The tube was then centrifuged for 1 minute at 1200 rpm and the supernatant removed. The protoplasts were resuspended in 1 milliliter incubation solution and transferred to a multi-well plate. The efficiency of genome editing was assessed by sequencing, as described below. The transfection efficiency was measured by detecting GFP fluorescence.

Editing efficiency was quantified as follows. Amplicons were sequenced using paired-end Illumina sequencing. Alignments generated from the reads were analyzed with CrispRVariants, which described and tallied all of the sequence alleles which differed within a 100 bp window centered on the cut site (Lindsay, H. et al. Nature Biotechnology 2016 34: 701-702). CrispRVariants reported the frequency of reads supporting each allele in number of reads of the total alignment. Different sequence alleles were categorized as 1) wildtype sequence, single nucleotide polymorphisms (SNPs), or sequencing artifacts, or 2) indel mutations. CrispRVariants automatically detected variants based on the type of mutation and its distance from the defined cut site, an additional filtering steps were used to remove any other sequence aberration that did not involve bases within 7 bp on either side of the predicted cut site. These alleles were placed in category 1. All sequencing alleles which had an insertion or deletion mutation that involved any base within 7 bp on either side of the cut site were determined to be indels and were placed in category 2. In the data analyzed, the frequencies reported for % indel are the sum of all frequencies of all sequencing alleles determined to be indels. The denominator for both frequencies is the sum of all reads which aligned to the reference amplicon. Editing efficiency was normalized for transfection efficiency. A Cas12 of a different family was used as a positive control. The editing results are summarized in Table 3 below.

TABLE 3
Summary of target editing results obtained with
different treatments of maize protoplasts.

| | | % Indel | | | | |
|---|---|---|---|---|---|---|
| Condition | Target | Rep1 | Rep2 | Rep3 | Average | STDEV |
| Modified crRNA | g5 | 0 | 0.03 | 0.01 | 0.01 | 0.01 |
| | g6 | 1.94 | 1.09 | 1.35 | 1.46 | 0.35 |
| | g9 | 0.46 | 0.71 | 0.84 | 0.67 | 0.16 |
| | Cas Control | 41.44 | 32.29 | 33.96 | 35.9 | 3.98 |
| Unmodified | g5 | 0 | 0 | 0 | 0 | 0 |
| crRNA | g6 | 0.05 | 0.05 | 0.13 | 0.08 | 0.04 |
| | g9 | 0.05 | 0.08 | 0.06 | 0.06 | 0.01 |
| | Cas Control | | | | | |
| Modified | g5 | 2.04 | 1.24 | 1.41 | 1.56 | 0.34 |
| crRNA + | g6 | 35.82 | 29.17 | 32.87 | 32.62 | 2.72 |
| scoutRNA | g9 | 26.12 | 22.78 | 19.08 | 22.66 | 2.88 |
| | Cas Control | | | | | |
| Negative Control | g5 | 0.01 | 0.01 | 0 | 0.01 | 0 |
| | g6 | 0.18 | 0.04 | 0.04 | 0.08 | 0.07 |
| | g9 | 0.01 | 0.01 | 0.04 | 0.02 | 0.01 |
| | Cas Control | 0.01 | 0.02 | 0 | 0.01 | 0.01 |

Exposure of the protoplasts to modified crRNA and scoutRNA in the presence of the Cas12d.15 (SEQ ID NO: 1) endonuclease resulted in editing frequencies for the g6 and g9 targets which were comparable to those observed with the Cas12 positive control, and detectible for the g5 target. In contrast, exposure of the protoplasts to modified crRNA in the presence of the Cas12d.15 (SEQ ID NO:1) endonuclease without scoutRNA resulted in significant reductions in editing frequencies for the g6 and g9 targets relative to the experiments where Cas12d.15 (SEQ ID NO:1) was used with both a modified crRNA and a scoutRNA.

### Example 8. Use of Single RNA Guides for Targeted Mutagenesis in Protoplasts with CRISPR/Cas12d

Maize protoplasts were made as described in Example 7.

Plasmid pIN3034 was used in transfection to express Cas12d. It has an expression cassette with promoter ZmEf1alpha promoter driving the expression of a coding sequence of Cas12d.15 fused at the C terminus to a nuclear localization signal PKKKRKV (SEQ ID NO: 9) and three HA epitope tags (SEQ ID NO: 10); 3′ of the coding sequence is the polyA addition site of HSP (SEQ ID NO: 11). pIN3034 also has a GFP expression cassette. This vector does not have an expression module for scoutRNA.

Protoplasts were transfected with pIN3c34 plasmid and sgRNA (same as single transcript RNA, abbreviated stgRNA below), or both scoutRNA and crRNAs as a positive control. Synthetic RNAs modified or Alt-Ra-modified were obtained from TDT (Coralville, IA, USA). ScoutRNA sequence was fused to crRNA to form single guides (sgRNAs) in different configurations, as shown below. The indicated sgRNAs had the g6 or g9 spacers (see Example 7).

The sequences used in the experiments are in Tables 4, 5, and 6 below. A positive control was used.

TABLE 4
Description of Experimental Designs

| Design | | |
|---|---|---|
| # | Description | crRNA |
| Design | Simple fusion of the scoutRNA with truncated DR | stgRNA_1 |
| 1 | preceding the spacer sequence | stgRNA_2 |
| Design | Similar to stgRNA_1 and stgRNA_2 but with full 5′ DR | stgRNA_3 |
| 2 | appended between termini of scout and start of spacer | stgRNA_4 |
| Design | scoutRNA extended with additional sequence from native | stgRNA_5 |
| 3 | locus as a linker, leading into truncated 5′ DR and spacer | stgRNA_6 |
| | sequence | |
| Design | scoutRNA extended with additional sequence from native | stgRNA_7 |
| 4 | locus as a linker, leading into full 5′ DR and spacer | stgRNA_8 |
| | sequence | |
| Design | scoutRNA extended with additional sequence forming | stgRNA_9 |
| 5 | hairpin, leading into truncated 5′ DR and spacer sequence | stgRNA_10 |
| Design | scoutRNA extended with additional sequence forming | stgRNA_11 |
| 6 | hairpin, leading into full 5′ DR and spacer sequence | stgRNA_12 |
| Design | scoutRNA extended with additional sequence forming | stgRNA_13 |
| 7 | pseudoknot, leading into truncated 5′ DR and spacer | stgRNA_14 |
| | sequence | |
| Design | scoutRNA extended with additional sequence forming | stgRNA_15 |
| 8 | pseudoknot, leading into full 5′ DR and spacer sequence | stgRNA_16 |
| Design | transcript consisting of full DR, Spacer sequence, full DR, | stgRNA_17 |
| 9 | 20 bp of native sequence 5′ of scoutRNA, and scoutRNA | stgRNA_18 |
| Design | Transcript comprising the sgRNA guide of SEQ ID NO: 5 | stgRNA_19 |
| 10 | substituted with a g6 or g9 spacer | stgRNA_20 |

TABLE 5
Description of Nucleic Acid Sequences

| Sequence | |
|---|---|
| Description | Sequence (SEQ ID NO:) |
| scoutRNA | CUUAGUUAAGGAUGUUCCAGGUUCUUUCGGGAGCCUUGG |
| Sequence | CCUUCUCCCUUAACCUAUGCC (SEQ ID NO: 3) |
| Truncated DR | GCGAUGAAGGC (SEQ ID NO: 12) |
| sequence | |
| Full DR sequence | acccguaaagcagagcgaugaaggc (SEQ ID NO: 13) |
| sequence | acuaaugauuaggaacacgg (SEQ ID NO: 14) |
| extension from | |
| native locus | |
| sequence | acuGCGGuAAuCCGCagaa (SEQ ID NO: 15) |
| extension to form | |
| hairpin | |
| sequence | aaccagaauaaauccugguucuggcauauaccaggaa (SEQ ID NO: 16) |
| extension to form | |
| pseudoknot | |
| Native locus 5′ of | acccguaaagcagagcgaugaaggcacauuggccgacuucgcugauaaaaau |
| scoutRNA (52 bp) | (SEQ ID NO: 17) |
| Native locus 5′ of | gagcgaugaaggcacauuggccgacuucgcugauaaaaau (SEQ ID NO: 18) |
| scoutRNA (40 bp) | |
| Native locus 5′ of | ccgacuucgcugauaaaaau (SEQ ID NO: 19) |
| scoutRNA (20 bp) | |
| g6 spacer | AuGAuGAAGGAGuGGGCG (SEQ ID NO: 20) |
| g9 Spacer | GAGCAAGAAGAuAACGGG (SEQ ID NO: 21) |

TABLE 6
Description of Guide RNAs and sequences of same

| | | Target/guide | |
|---|---|---|---|
| Number | Purpose/Rationale | name | Sequence (SEQ ID NO:) |
| stgRNA_1 | Simple fusion of the | Cas12d_M15_6 | CUUAGUUAAGGAUGUUCCA |
| | scoutRNA with | | GGUUCUUUCGGGAGCCUUG |
| | truncated DR | | GCCUUCUCCCUUAACCUAU |
| | preceding the spacer | | GCCGCGAUGAAGGCAuGAu |
| | sequence | | GAAGGAGuGGGCG (SEQ ID |
| | | | NO: 22) |
| stgRNA_2 | Simple fusion of the | Cas12d_M15_9 | CUUAGUUAAGGAUGUUCCA |
| | scoutRNA with | | GGUUCUUUCGGGAGCCUUG |
| | truncated DR | | GCCUUCUCCCUUAACCUAU |
| | preceding the spacer | | GCCGCGAUGAAGGCGAGCA |
| | sequence | | AGAAGAuAACGGG (SEQ ID |
| | | | NO: 23) |
| stgRNA_3 | Similar to stgRNA_1 | Cas12d_M15_6 | CUUAGUUAAGGAUGUUCCA |
| | and stgRNA_2 but | | GGUUCUUUCGGGAGCCUUG |
| | with full 5′ DR | | GCCUUCUCCCUUAACCUAU |
| | appended between | | GCCacccguaaagcagagcgaugaagg |
| | termini of scout and | | cAuGAuGAAGGAGuGGGCG |
| | start of spacer | | (SEQ ID NO: 24) |
| stgRNA_4 | Similar to stgRNA_1 | Cas12d_M15_9 | CUUAGUUAAGGAUGUUCCA |
| | and stgRNA_2 but | | GGUUCUUUCGGGAGCCUUG |
| | with full 5′ DR | | GCCUUCUCCCUUAACCUAU |
| | appended between | | GCCacccguaaagcagagcgaugaagg |
| | termini of scout and | | cGAGCAAGAAGAuAACGGG |
| | start of spacer | | (SEQ ID NO: 25) |
| stgRNA_5 | scoutRNA extended | Cas12d_M15_6 | CUUAGUUAAGGAUGUUCCA |
| | with additional | | GGUUCUUUCGGGAGCCUUG |
| | sequence from native | | GCCUUCUCCCUUAACCUAU |
| | locus as a linker, | | GCCacuaaugauuaggaacacggGCG |
| | leading into truncated | | AUGAAGGCAuGAuGAAGGA |
| | 5′ DR and spacer | | GuGGGCG (SEQ ID NO: 26) |
| | sequence | | |
| stgRNA_6 | scoutRNA extended | Cas12d_M15_9 | CUUAGUUAAGGAUGUUCCA |
| | with additional | | GGUUCUUUCGGGAGCCUUG |
| | sequence from native | | GCCUUCUCCCUUAACCUAU |
| | locus as a linker, | | GCCacuaaugauuaggaacacggGCG |
| | leading into truncated | | AUGAAGGCGAGCAAGAAG |
| | 5′ DR and spacer | | AuAACGGG (SEQ ID NO: 27) |
| | sequence | | |
| stgRNA_7 | scoutRNA extended | Cas12d_M15_6 | CUUAGUUAAGGAUGUUCCA |
| | with additional | | GGUUCUUUCGGGAGCCUUG |
| | sequence from native | | GCCUUCUCCCUUAACCUAU |
| | locus as a linker, | | GCCacuaaugauuaggaacacggaccc |
| | leading into full 5′ | | guaaagcagagcgaugaaggcAuGAu |
| | DR and spacer | | GAAGGAGuGGGCG (SEQ ID |
| | sequence | | NO: 28) |
| stgRNA_8 | scoutRNA extended | Cas12d_M15_9 | CUUAGUUAAGGAUGUUCCA |
| | with additional | | GGUUCUUUCGGGAGCCUUG |
| | sequence from native | | GCCUUCUCCCUUAACCUAU |
| | locus as a linker, | | GCCacuaaugauuaggaacacggaccc |
| | leading into full 5′ | | guaaagcagagcgaugaaggcGAGCA |
| | DR and spacer | | AGAAGAuAACGGG (SEQ ID |
| | sequence | | NO: 29) |
| stgRNA_9 | scoutRNA extended | Cas12d_M15_6 | CUUAGUUAAGGAUGUUCCA |
| | with additional | | GGUUCUUUCGGGAGCCUUG |
| | sequence forming | | GCCUUCUCCCUUAACCUAU |
| | hairpin, leading into | | GCCacuGCGGuAAuCCGCagaa |
| | truncated 5′ DR and | | GCGAUGAAGGCAuGAuGAA |
| | spacer sequence | | GGAGuGGGCG (SEQ ID NO: |
| | | | 30) |
| stgRNA_10 | scoutRNA extended | Cas12d_M15_9 | CUUAGUUAAGGAUGUUCCA |
| | with additional | | GGUUCUUUCGGGAGCCUUG |
| | sequence forming | | GCCUUCUCCCUUAACCUAU |
| | hairpin, leading into | | GCCacuGCGGuAAuCCGCagaa |
| | truncated 5′ DR and | | GCGAUGAAGGCGAGCAAGA |
| | spacer sequence | | AGAuAACGGG (SEQ ID NO: |
| | | | 31) |
| stgRNA_11 | scoutRNA extended | Cas12d_M15_6 | CUUAGUUAAGGAUGUUCCA |
| | with additional | | GGUUCUUUCGGGAGCCUUG |
| | sequence forming | | GCCUUCUCCCUUAACCUAU |
| | hairpin, leading into | | GCCacuGCGGuAAuCCGCagaa |
| | full 5′ DR and spacer | | acccguaaagcagagcgaugaaggcAu |
| | sequence | | GAuGAAGGAGuGGGCG (SEQ |
| | | | ID NO: 32) |
| stgRNA_12 | scoutRNA extended | Cas12d_M15_9 | CUUAGUUAAGGAUGUUCCA |
| | with additional | | GGUUCUUUCGGGAGCCUUG |
| | sequence forming | | GCCUUCUCCCUUAACCUAU |
| | hairpin, leading into | | GCCacuGCGGuAAuCCGCagaa |
| | full 5′ DR and spacer | | acccguaaagcagagcgaugaaggcGA |
| | sequence | | GCAAGAAGAuAACGGG |
| | | | (SEQ ID NO: 33) |
| stgRNA_13 | scoutRNA extended | Cas12d_M15_6 | CUUAGUUAAGGAUGUUCCA |
| | with additional | | GGUUCUUUCGGGAGCCUUG |
| | sequence forming | | GCCUUCUCCCUUAACCUAU |
| | pseudoknot, leading | | GCCaaccagaauaaauccugguucugg |
| | into truncated 5′ DR | | cauauaccaggaaGCGAUGAAGG |
| | and spacer sequence | | CAuGAuGAAGGAGuGGGCG |
| | | | (SEQ ID NO: 34) |
| stgRNA_14 | scoutRNA extended | Cas12d_M15_9 | CUUAGUUAAGGAUGUUCCA |
| | with additional | | GGUUCUUUCGGGAGCCUUG |
| | sequence forming | | GCCUUCUCCCUUAACCUAU |
| | pseudoknot, leading | | GCCaaccagaauaaauccugguucugg |
| | into truncated 5′ DR | | cauauaccaggaaGCGAUGAAGG |
| | and spacer sequence | | CGAGCAAGAAGAuAACGGG |
| | | | (SEQ ID NO: 35) |
| stgRNA_15 | scoutRNA extended | Cas12d_M15_6 | CUUAGUUAAGGAUGUUCCA |
| | with additional | | GGUUCUUUCGGGAGCCUUG |
| | sequence forming | | GCCUUCUCCCUUAACCUAU |
| | pseudoknot, leading | | GCCaaccagaauaaauccugguucugg |
| | into full 5′ DR and | | cauauaccaggaaacccguaaagcagagc |
| | spacer sequence | | gaugaaggcAuGAuGAAGGAGu |
| | | | GGGCG (SEQ ID NO: 36) |
| stgRNA_16 | scoutRNA extended | Cas12d_M15_9 | CUUAGUUAAGGAUGUUCC |
| | with additional | | AGGUUCUUUCGGGAGCCU |
| | sequence forming | | UGGCCUUCUCCCUUAACCU |
| | pseudoknot, leading | | AUGCCaaccagaauaaauccugguuc |
| | into full 5′ DR and | | uggcauauaccaggaaacccguaaagca |
| | spacer sequence | | gagcgaugaaggcGAGCAAGAAG |
| | | | AuAACGGG (SEQ ID NO: 37) |
| stgRNA_17 | transcript consisting | Cas12d_M15_6 | acccguaaagcagagcgaugaaggcAu |
| | of full DR, Spacer | | GAuGAAGGAGuGGGCGacccg |
| | sequence, full DR, | | uaaagcagagcgaugaaggcccgacuuc |
| | 20 bp of native | | gcugauaaaaauCUUAGUUAAG |
| | sequence 5′ of | | GAUGUUCCAGGUUCUUUC |
| | scoutRNA, and | | GGGAGCCUUGGCCUUCUCC |
| | scoutRNA | | CUUAACCUAUGCC (SEQ ID |
| | | | NO: 38) |
| stgRNA_18 | transcript consisting | Cas12d_M15_9 | acccguaaagcagagcgaugaaggcGA |
| | of full DR, Spacer | | GCAAGAAGAuAACGGGaccc |
| | sequence, full DR, | | guaaagcagagcgaugaaggcccgacuu |
| | 20 bp of native | | cgcugauaaaaauCUUAGUUAAG |
| | sequence 5′ of | | GAUGUUCCAGGUUCUUUC |
| | scoutRNA, and | | GGGAGCCUUGGCCUUCUCC |
| | scoutRNA | | CUUAACCUAUGCC (SEQ ID |
| | | | NO: 39) |
| stgRNA_19 | sgRNA guide of SEQ | | CUUAUUAAGGAUGUUCCA |
| | ID NO: 5 substituted | | GGUUCUUUCGGGAGCCUU |
| | with g6 spacer | | GGCCUUCUCCCUUAACUAU |
| | | | CGCGAUGAAGGCAuGAuGA |
| | | | AGGAGuGGGCG (SEQ ID NO: |
| | | | 40) |
| stgRNA_20 | sgRNA guide of SEQ | | CUUAUUAAGGAUGUUCCA |
| | ID NO: 5 substituted | | GGUUCUUUCGGGAGCCUU |
| | with g9 spacer | | GGCCUUCUCCCUUAACUAU |
| | | | CGCGAUGAAGGCGAGCAA |
| | | | GAAGAuAACGGG (SEQ ID |
| | | | NO: 41) |

The results of the protoplast gene editing experiments with the aforementioned RNA molecules and the Cas12d endonuclease of SEQ TD NO: 1 are set forth in Table 7. Editing efficiencies were calculated essentially as described in Example 7 and were normalized for transfection efficiency.

TABLE 7
Normalized Editing Efficiency

| | Loci “g6” Editing | | | | | Loci “g9” Editing | | | | |
|---|---|---|---|---|---|---|---|---|---|---|
| Design/ | Efficiency (% Indel) | | | | | Efficiency (% Indel) | | | | |
| Condition | Rep1 | Rep2 | Rep3 | Mean | StdDev | Rep1 | Rep2 | Rep3 | Mean | StdDev |
| 1 | 52.9 | 49.0 | 47.8 | 49.9 | 2.7 | 30.7 | 33.2 | 38.2 | 34.0 | 3.8 |
| 2 | 25.1 | 28.5 | 35.2 | 29.6 | 5.1 | 50.5 | 49.9 | 49.4 | 49.9 | 0.5 |
| 3 | 19.2 | 20.4 | 15.7 | 18.4 | 2.4 | 16.9 | 13.5 | 16.3 | 15.6 | 1.8 |
| 4 | 9.3 | 17.6 | 7.7 | 11.5 | 5.3 | 13.1 | 12.4 | 16.6 | 14.0 | 2.2 |
| 5 | 60.5 | 68.2 | 57.7 | 62.1 | 5.5 | 46.7 | 56.0 | 59.3 | 54.0 | 6.5 |
| 6 | 31.7 | 28.7 | 30.5 | 30.3 | 1.5 | 32.5 | 36.6 | 33.8 | 34.3 | 2.1 |
| 7 | 16.8 | 14.6 | 21.2 | 17.5 | 3.3 | 22.4 | 25.4 | 21.3 | 23.0 | 2.2 |
| 8 | 10.6 | 12.3 | 14.3 | 12.4 | 1.8 | 22.1 | 25.6 | 26.1 | 24.6 | 2.2 |
| 9 | 41.0 | 38.1 | 34.7 | 37.9 | 3.2 | 35.3 | 32.1 | 35.2 | 34.2 | 1.8 |
| [CR + SC] | 35.2 | 30.8 | | 33.0 | 3.1 | 23.0 | | | 23.0 | n/a |
| MT | 0.03 | 0.03 | 0.02 | 0.02 | 0.01 | | | | | |

Additional experiments to compare the editing efficiency of single guides in designs 1 and 5 of Table 3 with design 10 were completed essentially as described in Example 7. Editing efficiency was normalized for transfection efficiency. The sgRNAs for design 10 comprise sgRNAs where the spacer element of the sgRNA of SEQ ID NO:5 is substituted with the g6 (SEQ ID NO: 20) or the g9 spacer element (SEQ ID NO: 21). The design 10 sgRNA comprising the g6 spacer is stgRNA_19 (SEQ ID NO: 40). The design 10 sgRNA comprising the g9 spacer is stgRNA_20 (SEQ ID NO: 41).

TABLE 8
Comparison of Design 1, 5, and 10 editing efficiency

| | Loci “g6” Editing Efficiency | | | | | Loci “g9” Editing Efficiency | | | | |
|---|---|---|---|---|---|---|---|---|---|---|
| | (% Indel) | | | | | (% Indel) | | | | |
| | Rep1 | Rep2 | Rep3 | Mean | StdDev | Rep1 | Rep2 | Rep3 | Mean | StdDev |
| Design 1 | 45.3 | 56.5 | 52.4 | 51.4 | 5.6 | 45.9 | 33.4 | 28.1 | 35.8 | 9.2 |
| Design 5 | 59.9 | 83.8 | 67.8 | 70.5 | 12.2 | 51.4 | 51.5 | 44.6 | 49.2 | 3.9 |
| Design 10 | 53.6 | 58.4 | 46.3 | 52.8 | 6.1 | 56.5 | 69.0 | 76.8 | 67.4 | 10.3 |
| Mock | 0.04 | 0.02 | 0.06 | 0.04 | 0.02 | 0.02 | 0.02 | 0.00 | 0.01 | 0.01 |
| Transfection | | | | | | | | | | |

The present disclosure is not to be limited in scope by the specific embodiments described herein. Indeed, various modifications of the disclosure in addition to those described herein will become apparent to those skilled in the art from the foregoing description. Such modifications are intended to fall within the scope of the appended claims. All patents, applications, publications, test methods, literature, and other materials cited herein are hereby incorporated by reference in their entirety as if physically present in this specification.

What is claimed is:

1. A method for modifying expression of at least one chromosomal or extrachromosomal gene in a eukaryotic cell, said method comprising introducing into the cell:
(a) (i) a Clustered Regularly Interspersed Short Palindromic Repeats (CRISPR) RNA (crRNA) and a short-complementarity untranslated RNA (scoutRNA) or DNA encoding the crRNA and scoutRNA, or (ii) a chimeric cr/scoutRNA hybrid (sgRNA) or DNA encoding the sgRNA, wherein the crRNA or the sgRNA comprises a sequence complementary to a target sequence within the gene; and
(b) a CRISPR Cas12d endonuclease molecule, wherein said CRISPR/Cas12d endonuclease is capable of binding to the sequence to which the crRNA or sgRNA is targeted; wherein the eukaryotic cell is optionally a mammalian, yeast, fish, or plant cell.

2. The method of claim 1, wherein the crRNA comprises a repeat sequence of about 11 nucleotides and a spacer sequence of about 18 nucleotides, wherein the spacer sequence interacts with the target nucleic acid.

3. The method of claim 1, wherein: (i) the crRNA or scoutRNA or sgRNA comprises unconventional and/or modified nucleotides and/or comprises unconventional and/or modified backbone chemistries; (ii) wherein the scoutRNA or sgRNA comprises the RNA molecule of SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56; and/or (iii) wherein the sgRNA comprises the RNA molecule of SEQ ID NO: 5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59.

4. The method of claim 3, wherein crRNA or scoutRNA or sgRNA comprises one or more modifications selected from the group consisting of locked nucleic acid (LNA) bases, internucleotide phosphorothioate bonds in the backbone, 2′-O-Methyl RNA bases, unlocked nucleic acid (UNA) bases, 5-Methyl dC bases, 5-hydroxybutynl-2′-deoxyuridine bases, 5-nitro indole bases, deoxyinosine bases, 8-aza-7-deazaguanosine bases, dideoxy-T at the 5′ end, inverted dT at the 3′ end, and dideoxycytidine at the 3′ end.

5. The method of claim 1, wherein the crRNA, scoutRNA or sgRNA is introduced into the cell as a DNA molecule encoding said RNA and is operably linked to a promoter directing production of said RNA in the cell.

6. The method of claim 1, wherein the CRISPR/Cas12d endonuclease molecule comprises the amino acid sequence of SEQ ID NO: 1 or a sequence having at least 85% sequence identity to SEQ ID NO: 1.

7. The method of claim 6, wherein the CRISPR/Cas12d endonuclease molecule is capable of introducing a double stranded break or a single stranded break at, within, or near the sequence to which the crRNA or sgRNA is targeted.

8. The method of claim 1, wherein the CRISPR/Cas12d endonuclease molecule is a dCas12d.

9. The method of claim 8, wherein the dCas12d comprises a mutation of one or more of residues selected from the group consisting of D775, E971, D1198, C1053, C1056, C1186, and C1191 of SEQ ID NO: 1, optionally wherein the dCas12 molecule comprises one or more mutations selected from the group consisting of D775A, E971A, D1198A, C1053A, C1056A, C1186A, and C1191A of SEQ ID NO: 1.

10. The method of claim 1, wherein the CRISPR/Cas12d endonuclease molecule is modified so as to be active at a different temperature than its optimal temperature prior to modification.

11. The method of claim 10, wherein the modified CRISPR/Cas12d endonuclease molecule is active at temperatures suitable for growth and culture of eukaryotes or eukaryotic cells, wherein the eukaryotes are optionally mammals, yeasts, or plants and wherein the eukaryotic cell is optionally a mammalian, yeast, fungal, fish, or plant cell.

12. The method of claim 10, wherein the modified CRISPR/Cas12d endonuclease molecule is active at a temperature from about 20° C. to about 35° C.

13. The method of claim 12, wherein the modified CRISPR/Cas12d endonuclease molecule is active at a temperature from about 23° C. to about 32° C.

14. The method of claim 13, wherein the modified CRISPR/Cas12d endonuclease molecule is active at a temperature from about 25° C. to about 28° C.

15. The method of claim 1, wherein: (i) the CRISPR/Cas12d endonuclease molecule is delivered to the cell as a DNA molecule comprising a CRISPR/Cas12d endonuclease coding sequence operably linked to a promoter directing production of said CRISPR/Cas12d endonuclease in the cell; (ii) the crRNA and scoutRNA is delivered to the cell as a DNA molecule comprising one or more sequences encoding the crRNA and scoutRNA, wherein the one or more sequences encoding the crRNA and scoutRNA are operably linked to one or more promoter(s) directing production of the crRNA and scoutRNA in the cell; or (iii) both (i) and (ii).

16. The method of claim 15, wherein the DNA molecule is transiently present in the cell.

17. The method of claim 15, wherein the DNA molecule is stably incorporated into the nuclear or plastidic genomic sequence of the cell or a progenitor cell, thereby providing heritable expression of the CRISPR/Cas12d endonuclease molecule.

18. The method of claim 1, wherein the CRISPR/Cas12d endonuclease molecule is delivered to the cell as an mRNA molecule encoding said CRISPR/Cas12d endonuclease.

19. The method of claim 1, wherein the CRISPR/Cas12d endonuclease molecule is delivered to the cell as a protein.

20. The method of claim 1, wherein the CRISPR/Cas12d endonuclease molecule comprises one or more elements selected from the group consisting of localization signals, detection tags, detection reporters, and purification tags.

21. The method of claim 20, wherein the CRISPR/Cas12d endonuclease molecule comprises one or more localization signals.

22. The method of claim 1, wherein the CRISPR/Cas12d endonuclease molecule comprises at least one additional protein domain with enzymatic activity.

23. The method of claim 22, wherein the at least one additional protein domain has an enzymatic activity selected from the group consisting of exonuclease, helicase, repair of DNA double-stranded breaks, transcriptional (co-)activator, transcriptional (co-)repressor, methylase, demethylase, and any combinations thereof.

24. The method of claim 1, wherein the method comprises delivering a preassembled complex comprising the CRISPR/Cas12d endonuclease molecule loaded with the crRNA/scoutRNA or sgRNA prior to introduction into the cell.

25. The method of claim 5, wherein the promoter is selected from the group consisting of constitutive promoters, inducible promoters, and cell-type or tissue-type specific promoters.

26. The method of claim 5, wherein the promoter is activated by alternative splicing of a suicide exon.

27. The method of any one of claims 1-26, wherein the DNA or RNA is delivered to the cell by a method selected from the group consisting of microparticle bombardment, polyethylene glycol (PEG) mediated transformation, electroporation, pollen-tube mediated introduction into zygotes, and delivery mediated by one or more cell-penetrating peptides (CPPs).

28. The method of any one of claims 1-26, wherein the DNA is delivered to the cell by bacteria-mediated transformation.

29. The method of claim 28, wherein the DNA is delivered to the cell in a T-DNA, and wherein the delivery is via Agrobacterium or Ensifer.

30. The method of any one of claims 1-26, wherein the DNA or RNA is delivered to the cell by a virus.

31. The method of claim 30, wherein the virus is a geminivirus or a tobravirus.

32. The method of any one of claims 1-26, wherein the plant is monocotyledonous.

33. The method of any one of claims 1-26, wherein the plant is dicotyledonous.

34. The method of any one of claims 1-26, wherein:
(i) the eukaryotic cell is a mammalian cell optionally selected from the group consisting of a human, non-human primate, bovine, porcine, murine, canine, feline, equine, rodent, and an ungulate cell;
(ii) the eukaryotic cell is a yeast cell optionally selected from the group consisting of a Saccharomyces sp., Candida, Endomycopsis, Brettanomyces sp., Candida sp., Cryptococcus sp., Debaromyces sp, Hanseniaspora sp., Hansenula sp., Kluyveromyces sp., Pichia sp., Rhodotorula sp., Torulaspora sp., Schizosaccharomyces sp., and Zygosaccharomyces sp. cell;
(iii) the eukaryotic cell is a fungal cell optionally selected from the group consisting of a Aspergillus sp., Fusarium sp., Penicillium sp., Paecilomyces sp., Mucor sp., Rhizopus sp., and a Trichoderma sp. cell;
(iv) the eukaryotic cell is a fish cell optionally selected from the group consisting of a salmonid, cichlid, silurid, and cyprinid cell, or
(v) the eukaryotic cell is a plant cell optionally derived from a species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, and any variety or subspecies belonging to one of the aforementioned plants.

35. The method of any one of claims 1-26, wherein the target sequence is selected from the group consisting of an acetolactate synthase (ALS) gene, an enolpyruvylshikimate phosphate synthase gene (EPSPS) gene, male fertility genes, male sterility genes, female fertility genes, female sterility genes, male restorer genes, female restorer genes, genes associated with the traits of sterility, genes associated with the traits of fertility, genes associated with herbicide resistance, genes associated with herbicide tolerance, genes associated with fungal resistance, genes associated with viral resistance, genes associated with insect resistance, genes associated with drought tolerance, genes associated with chilling tolerance, genes associated with cold tolerance, genes associated with nitrogen use efficiency, genes associated with phosphorus use efficiency, genes associated with water use efficiency and genes associated with crop or biomass yield, and any mutants of such genes.

36. The method of claim 35, wherein male sterility gene is selected from the group consisting of MS45, MS26 and MSCA1.

37. A eukaryotic cell modified by the method of any one of claims 1-26, wherein the eukaryotic cell is optionally a mammalian, yeast, fungal, fish, or plant cell.

38. Cells, whole eukaryotic organisms, or progeny thereof derived from the cell of claim 37.

39. A composition comprising: (a) (i) a Clustered Regularly Interspersed Short Palindromic Repeats (CRISPR) RNA (crRNA) and a short-complementarity untranslated RNA (scoutRNA), or (ii) a chimeric cr/scoutRNA hybrid (sgRNA), wherein the crRNA or the sgRNA is targeted to a chromosomal or extrachromosomal plant gene sequence; and/or (b) a CRISPR/Cas12d endonuclease molecule, wherein said CRISPR/Cas12d endonuclease is capable of binding to the sequence to which the crRNA or sgRNA is targeted at temperatures suitable for growth and culture of a eukaryote or eukaryotic cell wherein the eukaryote is optionally a mammal, yeast, fungus, or plant and wherein the eukaryotic cell is optionally a mammalian, yeast, fungal, fish, or plant cell.

40. The composition of claim 39, wherein the crRNA comprises a repeat sequence of about 11 nucleotides and a spacer sequence of about 18 nucleotides, wherein the spacer sequence interacts with the target nucleic acid.

41. The composition of claim 39, wherein: (i) the crRNA or scoutRNA or sgRNA comprises unconventional and/or modified nucleotides and/or comprises unconventional and/or modified backbone chemistries; (ii) wherein the scoutRNA or sgRNA comprises the RNA molecule of SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56; and/or (iii) wherein the sgRNA comprises the RNA molecule of SEQ ID NO:5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59.

42. The composition of claim 41, wherein crRNA or scoutRNA or sgRNA comprises one or more modifications selected from the group consisting of locked nucleic acid (LNA) bases, internucleotide phosphorothioate bonds in the backbone, 2′-O-Methyl RNA bases, unlocked nucleic acid (UNA) bases, 5-Methyl dC bases, 5-hydroxybutynl-2′-deoxyuridine bases, 5-nitro indole bases, deoxyinosine bases, 8-aza-7-deazaguanosine bases, dideoxy-T at the 5′ end, inverted dT at the 3′ end, and dideoxycytidine at the 3′ end.

43. The composition of claim 39, wherein the CRISPR/Cas12d endonuclease molecule comprises the amino acid sequence of SEQ ID NO: 1 or a sequence having at least 85% sequence identity to SEQ ID NO: 1.

44. The composition of claim 39, wherein the CRISPR/Cas12d endonuclease molecule is capable of introducing a double stranded break or a single stranded break at, within, or near the sequence to which the crRNA or sgRNA is targeted.

45. The composition of claim 39, wherein the CRISPR/Cas12d endonuclease molecule is a dCas12d.

46. The composition of any one of claim 45, wherein the dCas12d comprises a mutation of one or more of residues D775, E971, D1198, C1053, C1056, C1186, and C1191 of SEQ ID NO: 1.

47. The composition of claim 39, wherein the CRISPR/Cas12d endonuclease molecule is modified so as to be active at a different temperature than its optimal temperature prior to modification.

48. The composition of claim 47, wherein the modified CRISPR/Cas12d endonuclease molecule is active at temperatures suitable for growth and culture of a eukaryote or eukaryotic cell, wherein the eukaryote is optionally a mammal, yeast, or plant and wherein the eukaryotic cell is optionally a mammalian, yeast, fungal, fish, or plant cell.

49. The composition of claim 47, wherein the modified CRISPR/Cas12d endonuclease molecule is active at a temperature from about 20° C. to about 35° C.

50. The composition of claim 49, wherein the modified CRISPR/Cas12d endonuclease molecule is active at a temperature from about 23° C. to about 32° C.

51. The composition of claim 50, wherein the modified CRISPR/Cas12d endonuclease molecule is active at a temperature from about 25° C. to about 28° C.

52. The composition of any one of claims 39-51, wherein the CRISPR/Cas12d endonuclease molecule comprises one or more elements selected from the group consisting of localization signals, detection tags, detection reporters, and purification tags.

53. The composition of any one of claims 39-51, wherein the CRISPR/Cas12d endonuclease molecule is modified to express nickase activity or to have a nucleic acid targeting activity without any nickase or endonuclease activity.

54. The composition of any one of claims 39-51, wherein the CRISPR/Cas12d endonuclease molecule comprises at least one additional protein domain with enzymatic activity.

55. The composition of claim 54, wherein the at least one additional protein domain has an enzymatic activity selected from the group consisting of exonuclease, helicase, repair of DNA double-stranded breaks, transcriptional (co-)activator, transcriptional (co-)repressor, methylase, demethylase, and any combinations thereof.

56. The composition of any one of claims 39-51, wherein the target sequence is a plant sequence selected from the group consisting of an acetolactate synthase (ALS) gene, an enolpyruvylshikimate phosphate synthase gene (EPSPS) gene, male fertility genes, male sterility genes, female fertility genes, female sterility genes, male restorer genes, female restorer genes, genes associated with the traits of sterility, genes associated with the traits of fertility, genes associated with herbicide resistance, genes associated with herbicide tolerance, genes associated with fungal resistance, genes associated with viral resistance, genes associated with insect resistance, genes associated with drought tolerance, genes associated with chilling tolerance, genes associated with cold tolerance, genes associated with nitrogen use efficiency, genes associated with phosphorus use efficiency, genes associated with water use efficiency and genes associated with crop or biomass yield, and any mutants of such genes.

57. The composition of claim 56, wherein male sterility gene is selected from the group consisting of MS45, MS26 and MSCA1.

58. The composition of any one of claims 39-51, wherein the plant is monocotyledonous.

59. The composition of any one of claims 39-51, wherein the plant is dicotyledonous.

60. The composition of any one of claims 39-51, wherein:
(i) the eukaryotic cell is a mammalian cell optionally selected from the group consisting of a human, non-human primate, bovine, porcine, murine, canine, feline, equine, rodent, and an ungulate cell;
(ii) the eukaryotic cell is a yeast cell optionally selected from the group consisting of a Saccharomyces sp., Candida, Endomycopsis, Brettanomyces sp., Candida sp., Cryptococcus sp., Debaromyces sp, Hanseniaspora sp., Hansenula sp., Kluyveromyces sp., Pichia sp., Rhodotorula sp., Torulaspora sp., Schizosaccharomyces sp., and Zygosaccharomyces sp. cell;
(iii) the eukaryotic cell is a fungal cell optionally selected from the group consisting of a Aspergillus sp., Fusarium sp., Penicillium sp., Paecilomyces sp., Mucor sp., Rhizopus sp., and a Trichoderma sp. cell;
(iv) the eukaryotic cell is a fish cell optionally selected from the group consisting of a salmonid, cichlid, silurid, and cyprinid cell; or
(v) the eukaryotic cell is a plant cell is derived from a species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, and any variety or subspecies belonging to one of the aforementioned plants.

61. A kit comprising: (a) (i) a Clustered Regularly Interspersed Short Palindromic Repeats (CRISPR) RNA (crRNA) and a short-complementarity untranslated RNA (scoutRNA), or (ii) a chimeric cr/scoutRNA hybrid (sgRNA), wherein the crRNA or the sgRNA is targeted to a sequence within a plant gene; (b) a CRISPR Cas12d endonuclease molecule, wherein said CRISPR Cas12d endonuclease is capable of introducing a double stranded break or a single stranded break at or near the sequence to which the crRNA or sgRNA is targeted at temperatures suitable for growth and culture of a eukaryote or eukaryotic cell, wherein the eukaryote is optionally a mammal, yeast, or plant and wherein the eukaryotic cell is optionally a mammalian, yeast, fungal, fish, or plant cell, and optionally (c) instructions for use.

62. A kit comprising: (a) (i) a nucleic acid molecule encoding Clustered Regularly Interspersed Short Palindromic Repeats (CRISPR) RNA (crRNA) and a short-complementarity untranslated RNA (scoutRNA), or (ii) a nucleic acid molecule encoding a chimeric cr/scoutRNA hybrid (sgRNA), wherein the crRNA or the sgRNA is targeted to a sequence within a plant gene; (b) a nucleic acid molecule encoding CRISPR Cas12d endonuclease molecule, wherein said CRISPR/Cas12d endonuclease is capable of introducing a double stranded break or a single stranded break at or near the sequence to which the crRNA or sgRNA is targeted at temperatures suitable for growth and culture of a eukaryote or eukaryotic cell, wherein the eukaryote is optionally a mammal, yeast, or plant and wherein the eukaryotic cell is optionally a mammalian, yeast, fungal, fish, or plant cell, and optionally (c) instructions for use.

63. A kit comprising: (a) (i) a nucleic acid molecule encoding Clustered Regularly Interspersed Short Palindromic Repeats (CRISPR) RNA (crRNA) and a nucleic acid molecule encoding a short-complementarity untranslated RNA (scoutRNA), or (ii) a nucleic acid molecule encoding a chimeric cr/scoutRNA hybrid (sgRNA), wherein the crRNA or the sgRNA is targeted to a sequence within a plant gene; (b) a nucleic acid molecule encoding CRISPR/Cas12d endonuclease molecule, wherein said CRISPR/Cas12d endonuclease is capable of introducing a double stranded break or a single stranded break at or near the sequence to which the crRNA or sgRNA is targeted at temperatures suitable for growth and culture of a eukaryote or eukaryotic cell, wherein the eukaryote is optionally a mammal, yeast, or plant and wherein the eukaryotic cell is optionally a mammalian, yeast, fungal, fish, or plant cell, and optionally (c) instructions for use.

64. The kit of any one of claims 61, 62, or 63, wherein: (i) the scoutRNA or sgRNA comprises the RNA molecule of SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56; or (ii) wherein the sgRNA comprises the RNA molecule of SEQ ID NO: 5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59.

65. A nucleic acid comprising an sgRNA or a DNA encoding an sgRNA for a Cas12d nuclease, wherein the sgRNA comprises (i) SEQ ID NO: 3, SEQ ID NO: 4, SEQ ID NO: 42, SEQ ID NO: 44, SEQ ID NO: 46, SEQ ID NO: 48, SEQ ID NO: 50, SEQ ID NO: 52, SEQ ID NO: 54, or SEQ ID NO: 56; or (ii) a spacer sequence directed to a heterologous eukaryotic DNA target sequence and a scout RNA comprising an RNA molecule of SEQ ID NO: 5, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 58, or SEQ ID NO: 59; optionally wherein the nucleic acid is isolated.

66. A dCas12d molecule comprising a mutation of one or more of residues selected from the group consisting of D775, E971, D1198, C1053, C1056, C1186, and C1191 of SEQ ID NO: 1, optionally wherein the dCas12 molecule comprises one or more mutations selected from the group consisting of D775A, E971A, D1198A, C1053A, C1056A, C1186A, and C1191A of SEQ ID NO: 1.


SEQUENCE LISTING (59 sequences)

SEQ ID NO: 1 - protein, 1258 aa, Unknown
  bacterial
MNEKKTATQRRNARRRRGERARTKSQELRGYRLHDARIEFSGGLGSMRTVKVELLNPDSSREDPQRGQGLQGKVAKAVFDDYRALYGPMNIEDYLSDPDCPSFLGLWVKAVCLGVIMSRKTATDFGELRGGSKSGQAFDSIPEHLRRQLIKLKWLDWYDKGIRKSSSKASRLKSLTDVFANPKQPDQGVMAAWEQGEKLAESSRDIAALGRREFKDKLFAIPPPTSSVVLDDDVKATKVSRDWQWAVDPQFKLPSTDLDITRALEEVDRQWFERLGNNRGMVQQFFAIGDNGNHLNNGLFGHFFASIRSANLADIVAEMGTAFGFSAEERDIVRQRLETLHEYAQGLPEKPVLASRWAEYRTDMTAKLGSWYSNRTSKGAASITQVWGTINTETGEVKDDGLVRTLENIQSDLPDSCSIKEGILQETLDFIGDRRSSTDRAFTDELELYLATLRSDLNTWCQEQSALWEEKQRQVATPASDEKSKKADNPWAGKGSKTDKWLGALHTRIQSSPLFWGVDKLELWKTLANLKQAIRDEIDKLNEQVEVFGRSAYDEPVGKDADSGEGDRRVDQLSYLSARLGDQAHEEVRQRLDAIALALGVKFSERDDLHRFFVSSRARRRAALLAMPNTITVGKLRELADLTPLWERIKKKPEEPRLLADTVALSKVVNSACASRANPSDQIELTTIHSRLDGYSKNIGHTEFISRATVQSTNGAQNTVALDSLVSPRLFYYNFPNIVESAEPHVSHLEVATRGNLGSFEEFAAKEHRTFDRENPQKDSRNRIDSVNPLAVASSRYQIQFFTWWAGLHRSKETALEVGGSFTIAERQVRLDWSQEKPQAVVSEELRVFVSQPFTIVPDDKKRPATSGTRYIGVDIGEYGLAWSCWEFAPGYWNGSVVNPSKVTCLDYGFLAEPGQRRIVERVKKLRESQATKTFTSPDTYIARLRENVVATYQAQLEALMMAYNAQLVFESEISAFETGGNRVKKIYDAIKRSSVFGRSDAEATDNNQHWGKNGNRSSVKDPDKLRLNEAGQVAARVPWAEPVSAWMTSQTCSACGRVYVRAYRGKNSNEPDSGATGEVRYFDNKQQKILTKTIGADTVWVTDQERKEFERGVYNAMRPNAFMPDGRWTAAGEILEAALKSRGTLDGGRGFAGLHLTSKAQVHEYIEGTGKSHRDAHGNSAIFICPYTDCGHIADADLQASYNIALRGFAYAIVRKKHPELFAGSGSSTDGDEGGGKKPQQKQAFIDEIVRAAGRAS

SEQ ID NO: 2 - protein, 1298 aa, Artificial
  synthetic
MKRPAATKKAGQAKKKKGGSGGGSGMNEKKTATQRRNARRRRGERARTKSQELRGYRLHDARIEFSGGLGSMRTVKVELLNPDSSREDPQRGQGLQGKVAKAVFDDYRALYGPMNIEDYLSDPDCPSFLGLWVKAVCLGVIMSRKTATDFGELRGGSKSGQAFDSIPEHLRRQLIKLKWLDWYDKGIRKSSSKASRLKSLTDVFANPKQPDQGVMAAWEQGEKLAESSRDIAALGRREFKDKLFAIPPPTSSVVLDDDVKATKVSRDWQWAVDPQFKLPSTDLDITRALEEVDRQWFERLGNNRGMVQQFFAIGDNGNHLNNGLFGHFFASIRSANLADIVAEMGTAFGFSAEERDIVRQRLETLHEYAQGLPEKPVLASRWAEYRTDMTAKLGSWYSNRTSKGAASITQVWGTINTETGEVKDDGLVRTLENIQSDLPDSCSIKEGILQETLDFIGDRRSSTDRAFTDELELYLATLRSDLNTWCQEQSALWEEKQRQVATPASDEKSKKADNPWAGKGSKTDKWLGALHTRIQSSPLFWGVDKLELWKTLANLKQAIRDEIDKLNEQVEVFGRSAYDEPVGKDADSGEGDRRVDQLSYLSARLGDQAHEEVRQRLDAIALALGVKFSERDDLHRFFVSSRARRRAALLAMPNTITVGKLRELADLTPLWERIKKKPEEPRLLADTVALSKVVNSACASRANPSDQIELTTIHSRLDGYSKNIGHTEFISRATVQSTNGAQNTVALDSLVSPRLFYYNFPNIVESAEPHVSHLEVATRGNLGSFEEFAAKEHRTFDRENPQKDSRNRIDSVNPLAVASSRYQIQFFTWWAGLHRSKETALEVGGSFTIAERQVRLDWSQEKPQAVVSEELRVFVSQPFTIVPDDKKRPATSGTRYIGVDIGEYGLAWSCWEFAPGYWNGSVVNPSKVTCLDYGFLAEPGQRRIVERVKKLRESQATKTFTSPDTYIARLRENVVATYQAQLEALMMAYNAQLVFESEISAFETGGNRVKKIYDAIKRSSVFGRSDAEATDNNQHWGKNGNRSSVKDPDKLRLNEAGQVAARVPWAEPVSAWMTSQTCSACGRVYVRAYRGKNSNEPDSGATGEVRYFDNKQQKILTKTIGADTVWVTDQERKEFERGVYNAMRPNAFMPDGRWTAAGEILEAALKSRGTLDGGRGFAGLHLTSKAQVHEYIEGTGKSHRDAHGNSAIFICPYTDCGHIADADLQASYNIALRGFAYAIVRKKHPELFAGSGSSTDGDEGGGKKPQQKQAFIDEIVRAAGRASGGSGGGSGPKKKRKV

SEQ ID NO: 3 - RNA, 60 nt, Artificial
  synthetic
cuuaguuaaggauguuccagguucuuucgggagccuuggccuucucccuuaaccuaugcc

SEQ ID NO: 4 - RNA, 29 nt, Artificial
  synthetic
  misc_feature (12)..(29): n is a, c, g, or u
gcgaugaaggcnnnnnnnnnnnnnnnnnn

SEQ ID NO: 5 - RNA, 85 nt, Artificial
  synthetic
  misc_feature (68)..(85): n is a, c, g, or u
cuuauuaaggauguuccagguucuuucgggagccuuggccuucucccuuaacuaucgcgaugaaggcnnnnnnnnnnnnnnnnnn

SEQ ID NO: 6 - DNA, 18 bp, Artificial
  synthetic
gagcacggaacgagcaag

SEQ ID NO: 7 - DNA, 18 bp, Artificial
  synthetic
atgatgaaggagtgggcg

SEQ ID NO: 8 - DNA, 18 bp, Artificial
  synthetic
gagcaagaagataacggg

SEQ ID NO: 9 - protein, 7 aa, Artificial
  synthetic
PKKKRKV

SEQ ID NO: 10 - protein, 32 aa, Artificial
  synthetic
YPYDVPDYAGYPYDVPDYAGSYPYDVPDYATS

SEQ ID NO: 11 - DNA, 250 bp, Artificial
  synthetic
atatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataag

SEQ ID NO: 12 - RNA, 11 nt, Artificial
  synthetic
gcgaugaaggc

SEQ ID NO: 13 - RNA, 25 nt, Artificial
  synthetic
acccguaaagcagagcgaugaaggc

SEQ ID NO: 14 - RNA, 20 nt, Artificial
  synthetic
acuaaugauuaggaacacgg

SEQ ID NO: 15 - RNA, 19 nt, Artificial
  synthetic
acugcgguaauccgcagaa

SEQ ID NO: 16 - RNA, 37 nt, Artificial
  synthetic
aaccagaauaaauccugguucuggcauauaccaggaa

SEQ ID NO: 17 - RNA, 52 nt, Artificial
  synthetic
acccguaaagcagagcgaugaaggcacauuggccgacuucgcugauaaaaau

SEQ ID NO: 18 - RNA, 40 nt, Artificial
  synthetic
gagcgaugaaggcacauuggccgacuucgcugauaaaaau

SEQ ID NO: 19 - RNA, 20 nt, Artificial
  synthetic
ccgacuucgcugauaaaaau

SEQ ID NO: 20 - RNA, 18 nt, Artificial
  synthetic
augaugaaggagugggcg

SEQ ID NO: 21 - RNA, 18 nt, Artificial
  synthetic
gagcaagaagauaacggg

SEQ ID NO: 22 - RNA, 89 nt, Artificial
  synthetic
cuuaguuaaggauguuccagguucuuucgggagccuuggccuucucccuuaaccuaugccgcgaugaaggcaugaugaaggagugggcg

SEQ ID NO: 23 - RNA, 89 nt, Artificial
  synthetic
cuuaguuaaggauguuccagguucuuucgggagccuuggccuucucccuuaaccuaugccgcgaugaaggcgagcaagaagauaacggg

SEQ ID NO: 24 - RNA, 103 nt, Artificial
  synthetic
cuuaguuaaggauguuccagguucuuucgggagccuuggccuucucccuuaaccuaugccacccguaaagcagagcgaugaaggcaugaugaaggagugggcg

SEQ ID NO: 25 - RNA, 103 nt, Artificial
  synthetic
cuuaguuaaggauguuccagguucuuucgggagccuuggccuucucccuuaaccuaugccacccguaaagcagagcgaugaaggcgagcaagaagauaacggg

SEQ ID NO: 26 - RNA, 109 nt, Artificial
  synthetic
cuuaguuaaggauguuccagguucuuucgggagccuuggccuucucccuuaaccuaugccacuaaugauuaggaacacgggcgaugaaggcaugaugaaggagugggcg

SEQ ID NO: 27 - RNA, 109 nt, Artificial
  synthetic
cuuaguuaaggauguuccagguucuuucgggagccuuggccuucucccuuaaccuaugccacuaaugauuaggaacacgggcgaugaaggcgagcaagaagauaacggg

SEQ ID NO: 28 - RNA, 123 nt, Artificial
  synthetic
cuuaguuaaggauguuccagguucuuucgggagccuuggccuucucccuuaaccuaugccacuaaugauuaggaacacggacccguaaagcagagcgaugaaggcaugaugaaggagugggcg

SEQ ID NO: 29 - RNA, 123 nt, Artificial
  synthetic
cuuaguuaaggauguuccagguucuuucgggagccuuggccuucucccuuaaccuaugccacuaaugauuaggaacacggacccguaaagcagagcgaugaaggcgagcaagaagauaacggg

SEQ ID NO: 30 - RNA, 108 nt, Artificial
  synthetic
cuuaguuaaggauguuccagguucuuucgggagccuuggccuucucccuuaaccuaugccacugcgguaauccgcagaagcgaugaaggcaugaugaaggagugggcg

SEQ ID NO: 31 - RNA, 108 nt, Artificial
  synthetic
cuuaguuaaggauguuccagguucuuucgggagccuuggccuucucccuuaaccuaugccacugcgguaauccgcagaagcgaugaaggcgagcaagaagauaacggg

SEQ ID NO: 32 - RNA, 122 nt, Artificial
  synthetic
cuuaguuaaggauguuccagguucuuucgggagccuuggccuucucccuuaaccuaugccacugcgguaauccgcagaaacccguaaagcagagcgaugaaggcaugaugaaggagugggcg

SEQ ID NO: 33 - RNA, 122 nt, Artificial
  synthetic
cuuaguuaaggauguuccagguucuuucgggagccuuggccuucucccuuaaccuaugccacugcgguaauccgcagaaacccguaaagcagagcgaugaaggcgagcaagaagauaacggg

SEQ ID NO: 34 - RNA, 126 nt, Artificial
  synthetic
cuuaguuaaggauguuccagguucuuucgggagccuuggccuucucccuuaaccuaugccaaccagaauaaauccugguucuggcauauaccaggaagcgaugaaggcaugaugaaggagugggcg

SEQ ID NO: 35 - RNA, 126 nt, Artificial
  synthetic
cuuaguuaaggauguuccagguucuuucgggagccuuggccuucucccuuaaccuaugccaaccagaauaaauccugguucuggcauauaccaggaagcgaugaaggcgagcaagaagauaacggg

SEQ ID NO: 36 - RNA, 140 nt, Artificial
  synthetic
cuuaguuaaggauguuccagguucuuucgggagccuuggccuucucccuuaaccuaugccaaccagaauaaauccugguucuggcauauaccaggaaacccguaaagcagagcgaugaaggcaugaugaaggagugggcg

SEQ ID NO: 37 - RNA, 140 nt, Artificial
  synthetic
cuuaguuaaggauguuccagguucuuucgggagccuuggccuucucccuuaaccuaugccaaccagaauaaauccugguucuggcauauaccaggaaacccguaaagcagagcgaugaaggcgagcaagaagauaacggg

SEQ ID NO: 38 - RNA, 148 nt, Artificial
  synthetic
acccguaaagcagagcgaugaaggcaugaugaaggagugggcgacccguaaagcagagcgaugaaggcccgacuucgcugauaaaaaucuuaguuaaggauguuccagguucuuucgggagccuuggccuucucccuuaaccuaugcc

SEQ ID NO: 39 - RNA, 148 nt, Artificial
  synthetic
acccguaaagcagagcgaugaaggcgagcaagaagauaacgggacccguaaagcagagcgaugaaggcccgacuucgcugauaaaaaucuuaguuaaggauguuccagguucuuucgggagccuuggccuucucccuuaaccuaugcc

SEQ ID NO: 40 - RNA, 85 nt, Artificial
  synthetic
cuuauuaaggauguuccagguucuuucgggagccuuggccuucucccuuaacuaucgcgaugaaggcaugaugaaggagugggcg

SEQ ID NO: 41 - RNA, 85 nt, Artificial
  synthetic
cuuauuaaggauguuccagguucuuucgggagccuuggccuucucccuuaacuaucgcgaugaaggcgagcaagaagauaacggg

SEQ ID NO: 42 - RNA, 71 nt, Artificial
  synthetic
cuuaguuaaggauguuccagguucuuucgggagccuuggccuucucccuuaaccuaugccgcgaugaaggc

SEQ ID NO: 43 - RNA, 89 nt, Artificial
  synthetic
  misc_feature (72)..(89): n is a, c, g, or u
cuuaguuaaggauguuccagguucuuucgggagccuuggccuucucccuuaaccuaugccgcgaugaaggcnnnnnnnnnnnnnnnnnn

SEQ ID NO: 44 - RNA, 85 nt, Artificial
  synthetic
cuuaguuaaggauguuccagguucuuucgggagccuuggccuucucccuuaaccuaugccacccguaaagcagagcgaugaaggc

SEQ ID NO: 45 - RNA, 103 nt, Artificial
  synthetic
  misc_feature (86)..(103): n is a, c, g, or u
cuuaguuaaggauguuccagguucuuucgggagccuuggccuucucccuuaaccuaugccacccguaaagcagagcgaugaaggcnnnnnnnnnnnnnnnnnn

SEQ ID NO: 46 - RNA, 91 nt, Artificial
  synthetic
cuuaguuaaggauguuccagguucuuucgggagccuuggccuucucccuuaaccuaugccacuaaugauuaggaacacgggcgaugaaggc

SEQ ID NO: 47 - RNA, 109 nt, Artificial
  synthetic
  misc_feature (92)..(109): n is a, c, g, or u
cuuaguuaaggauguuccagguucuuucgggagccuuggccuucucccuuaaccuaugccacuaaugauuaggaacacgggcgaugaaggcnnnnnnnnnnnnnnnnnn

SEQ ID NO: 48 - RNA, 105 nt, Artificial
  synthetic
cuuaguuaaggauguuccagguucuuucgggagccuuggccuucucccuuaaccuaugccacuaaugauuaggaacacggacccguaaagcagagcgaugaaggc

SEQ ID NO: 49 - RNA, 123 nt, Artificial
  synthetic
  misc_feature (106)..(123): n is a, c, g, or u
cuuaguuaaggauguuccagguucuuucgggagccuuggccuucucccuuaaccuaugccacuaaugauuaggaacacggacccguaaagcagagcgaugaaggcnnnnnnnnnnnnnnnnnn

SEQ ID NO: 50 - RNA, 90 nt, Artificial
  synthetic
cuuaguuaaggauguuccagguucuuucgggagccuuggccuucucccuuaaccuaugccacugcgguaauccgcagaagcgaugaaggc

SEQ ID NO: 51 - RNA, 108 nt, Artificial
  synthetic
  misc_feature (91)..(108): n is a, c, g, or u
cuuaguuaaggauguuccagguucuuucgggagccuuggccuucucccuuaaccuaugccacugcgguaauccgcagaagcgaugaaggcnnnnnnnnnnnnnnnnnn

SEQ ID NO: 52 - RNA, 104 nt, Artificial
  synthetic
cuuaguuaaggauguuccagguucuuucgggagccuuggccuucucccuuaaccuaugccacugcgguaauccgcagaaacccguaaagcagagcgaugaaggc

SEQ ID NO: 53 - RNA, 116 nt, Artificial
  synthetic
  misc_feature (105)..(116): n is a, c, g, or u
cuuaguuaaggauguuccagguucuuucgggagccuuggccuucucccuuaaccuaugccacugcgguaauccgcagaaacccguaaagcagagcgaugaaggcnnnnnnnnnnnn

SEQ ID NO: 54 - RNA, 108 nt, Artificial
  synthetic
cuuaguuaaggauguuccagguucuuucgggagccuuggccuucucccuuaaccuaugccaaccagaauaaauccugguucuggcauauaccaggaagcgaugaaggc

SEQ ID NO: 55 - RNA, 126 nt, Artificial
  synthetic
  misc_feature (109)..(126): n is a, c, g, or u
cuuaguuaaggauguuccagguucuuucgggagccuuggccuucucccuuaaccuaugccaaccagaauaaauccugguucuggcauauaccaggaagcgaugaaggcnnnnnnnnnnnnnnnnnn

SEQ ID NO: 56 - RNA, 122 nt, Artificial
  synthetic
cuuaguuaaggauguuccagguucuuucgggagccuuggccuucucccuuaaccuaugccaaccagaauaaauccugguucuggcauauaccaggaaacccguaaagcagagcgaugaaggc

SEQ ID NO: 57 - RNA, 140 nt, Artificial
  synthetic
  misc_feature (123)..(140): n is a, c, g, or u
cuuaguuaaggauguuccagguucuuucgggagccuuggccuucucccuuaaccuaugccaaccagaauaaauccugguucuggcauauaccaggaaacccguaaagcagagcgaugaaggcnnnnnnnnnnnnnnnnnn

SEQ ID NO: 58 - RNA, 148 nt, Artificial
  synthetic
  misc_feature (26)..(43): n is a, c, g, or u
acccguaaagcagagcgaugaaggcnnnnnnnnnnnnnnnnnnacccguaaagcagagcgaugaaggcccgacuucgcugauaaaaaucuuaguuaaggauguuccagguucuuucgggagccuuggccuucucccuuaaccuaugcc

SEQ ID NO: 59 - RNA, 168 nt, Artificial
  synthetic
  misc_feature (26)..(43): n is a, c, g, or u
acccguaaagcagagcgaugaaggcnnnnnnnnnnnnnnnnnnacccguaaagcagagcgaugaaggcgagcgaugaaggcacauuggccgacuucgcugauaaaaaucuuaguuaaggauguuccagguucuuucgggagccuuggccuucucccuuaaccuaugcc